(19) United States
(12) Patent Application Publication
(10) Pub. No.: US 2002/0090619 A1
(43) Pub. Date: Jul. 11, 2002

(54) Methods for identifying mitochondrial divalent cation transporters

(76) Inventors: Douglas R. Pfeiffer, Delaware, OH (US); Anne N. Murphy, Encinitas, CA (US); Dennis W. Jung, Frederick, MD (US); Patrick C. Bradshaw, East Peoria, IL (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/894,991

(22) Filed: Jun. 29, 2001

Related U.S. Application Data
(60) Provisional application No. 60/215,737, filed on Jun. 29, 2000.

(51) Int. Cl. C12Q 1/68; C12Q 1/00

(52) U.S. Cl. 435/6; 435/4

(57) ABSTRACT

The invention relates to methods for identifying nucleic acid molecules that encode mitochondrial divalent cation transporter polypeptides, which in preferred embodiments are mitochondrial calcium uniporters. Preferred methods employ host cells for expression screening of nucleic acid libraries encoding candidate cation transporters, where the host cells comprise a mitochondrially targeted divalent cation-sensitive indicator molecule. In certain preferred embodiments host cells comprise a mitochondrially targeted esterase that is capable of cleaving a divalent cation sensitive indicator molecule precursor to provide a divalent cation sensitive indicator molecule, or an expression construct directing the expression of such esterase. Host cells that lack endogenous calcium uniporter activity, or that exhibit impaired cell growth in the presence of cytostatic Ca²⁺ concentrations, are also described.

### CROSS-REFERENCE TO RELATED APPLICATION

This application claims the benefit of U.S. Provisional Patent Application No. 60/215,737, filed Jun. 29, 2000, which is incorporated herein by reference in its entirety.

### TECHNICAL FIELD

The invention relates generally to compositions and methods for regulating divalent cations in biological systems, and in particular to mitochondrial involvement in intracellular calcium homeostasis. The invention thus relates in part to methods for use in identifying a mitochondrial calcium uniporter. The invention also relates to screening methods for use in identifying agents that alter mitochondrial regulation of intracellular calcium.

### BACKGROUND OF THE INVENTION

Mitochondria are organelles that are the main energy source in cells of higher organisms. These organelles provide direct and indirect biochemical regulation of a wide array of respiratory, oxidative and metabolic processes in cells, including metabolic energy production, aerobic respiration and intracellular calcium regulation. For example, mitochondria are the site of electron transport chain (ETC) activity. The ETC is not only involved in oxidative phosphorylation, which produces metabolic energy in the form of adenosine triphosphate (ATP), but also underlies the pivotal mitochondrial role in maintaining intracellular calcium homeostasis. These processes require the maintenance of a mitochondrial membrane electrochemical potential, and defects in such membrane potential can result in a variety of disorders. Accordingly, for normal cellular function, components involved in these processes must be properly localized in the mitochondria and the mitochondrial structure must be intact.

Mitochondrial ultrastructural characterization reveals the presence of an outer mitochondrial membrane that serves as an interface between the organelle and the cytosol, a highly folded inner mitochondrial membrane that appears to form multiple attachments to the outer membrane, and an intermembrane space between the two mitochondrial membranes. The subcompartment surrounded by the inner mitochondrial membrane is commonly referred to as the mitochondrial matrix. (For a review, see, e.g., Emster et al., 1981, J. Cell Biol. 91:227s.) The cristae, originally postulated to occur as infoldings of the inner mitochondrial membrane, have recently been characterized using three-dimensional electron tomography as also including tube-like conduits that may form networks, which may be connected to the inner membrane by open, circular (30 nm diameter) junctions (Perkins et al., 1997, Journal of Structural Biology 119:260). While the outer membrane is freely permeable to ionic and non-ionic solutes having molecular weights less than about ten kilodaltons, the inner mitochondrial membrane exhibits selective and regulated permeability for cytoplasmically synthesized mitochondrial proteins and many small molecules, including certain cations, and is impermeable to large (greater than about 10 kD) molecules.

Three of the four mitochondrial multisubunit protein complexes (Complexes I, III and IV) that mediate ETC activity are localized to the inner mitochondrial membrane. The remaining ETC complex (Complex II) is also associated with the matrix aspect of the inner mitochondrial membrane but does not occur in transmembrane orientation. Mitochondrial multisubunit protein Complex V (ATP synthase), while not part of the ETC, acts as a conduit for protons pumped out of the mitochondria to reenter the mitochondrial matrix. In at least three distinct chemical reactions known to take place within the ETC, protons are moved from the mitochondrial matrix, across the inner membrane, to the intermembrane space. This disequilibrium of charged species creates an electrochemical membrane potential of approximately 220 mV referred to as the “protonmotive force” (PMF). The PMF, which is often represented by the notation ×p, corresponds to the sum of the electric potential (ΔΨm) and the pH differential (ΔpH) across the inner membrane according to the equation

Δp=ΔΨm−ZΔpH

wherein Z stands for −2.303 RT/F. The value of Z is −59 at 25° C. when Δp and ΔΨm are expressed in mV and ΔpH is expressed in pH units (see, e.g., Emster et al., J. Cell Biol. 91 :227s, 1981, and references cited therein).

ΔΨm provides the energy for phosphorylation of adenosine diphosphate (ADP) to yield ATP by ETC Complex V, a process that is coupled stoichiometrically with transport of a proton into the matrix. ΔΨm is also the driving force for the influx of cytosolic Ca²⁺ into the mitochondrion. Under normal metabolic conditions, the inner membrane is largely impermeable to proton movement from the intermembrane space into the matrix, leaving ETC Complex V as the primary means whereby protons can return to the matrix. When, however, the integrity of the inner mitochondrial membrane is compromised, as occurs during mitochondrial permeability transition (MPT) that accompanies certain diseases associated with altered mitochondrial function, protons are able to bypass the conduit of Complex V without generating ATP, thereby uncoupling respiration (i.e., ETC activity) from ATP production. During MPT, ΔΨm collapses and mitochondrial membranes lose the ability to selectively regulate permeability to solutes both small (e.g., ionic Ca²⁺, Na⁺, K⁺ and H⁺) and large (e.g., proteins). Loss of mitochondrial potential also appears to be a critical event in the progression of diseases associated with altered mitochondrial function, including degenerative diseases such as Alzheimer's Disease; diabetes mellitus; Parkinson's Disease; Huntington's disease; dystonia; Leber's hereditary optic neuropathy; schizophrenia; mitochondrial encephalopathy, lactic acidosis, and stroke (MELAS); cancer; psoriasis; hyperproliferative disorders; mitochondrial diabetes and deafness (MIDD) and myoclonic epilepsy ragged red fiber syndrome, as well as numerous other mitochondria associated diseases. The extensive list of additional diseases associated with altered mitochondrial function continues to expand as aberrant mitochondrial or mitonuclear (i.e., related to direct or indirect interactions between mitochondria and cell nucleus) activities are implicated in particular disease processes.

Normal alterations of intramitochondrial Ca²⁺ are associated with normal metabolic regulation (Dykens, 1998 in Mitochondria & Free Radicals in Neurodegenerative Diseases, Beal, Howell and Bodis-Wollner, Eds., Wiley-Liss, New York, pp. 29-55; Radi et al., 1998 in Mitochondria & Free Radicals in Neurodegenerative Diseases, Beal, Howell and Bodis-Wollner, Eds., Wiley-Liss, New York, pp. 57-89; Gunter and Pfeiffer, 1990, Am. J. Physiol. 27: C755; Gunter et al., Am. J. Physiol. 267:313, 1994). For example, fluctuating levels of mitochondrial free Ca²⁺ may be responsible for regulating oxidative metabolism in response to increased ATP utilization, via allosteric regulation of enzymes (reviewed by Crompton and Andreeva, Basic Res. Cardiol. 88:513-523, 1993); and the glycerophosphate shuttle (Gunter and Gunter, J. Bioenerg. Biomembr. 26:471, 1994).

Normal mitochondrial function includes regulation of cytosolic free calcium levels by sequestration of excess Ca²⁺ within the mitochondrial matrix. Depending on cell type, cytosolic Ca²⁺ concentration is typically 50-100 nM. In normally functioning cells, when Ca²⁺ levels reach 200-300 nM, mitochondria begin to accumulate Ca²⁺ as a function of the equilibrium between influx via a Ca²⁺ uniporter in the inner mitochondrial membrane and Ca²⁺ efflux via both Na⁺ dependent and Na⁺ independent calcium carriers. The low affinity of this rapid uniporter mechanism suggests that the primary uniporter function may be to lower cytosolic Ca²⁺ in response to pathological elevation of cytosolic free calcium levels, which may result from ATP depletion and/or abnormal calcium influx across the plasma membrane (Gunter and Gunter, J. Bioenerg. Biomembr. 26:471, 1994; Gunter et al., Am. J. Physiol. 267:313, 1994). In certain instances, such perturbation of intracellular calcium homeostasis is a feature of diseases associated with altered mitochondrial function, regardless of whether the calcium regulatory dysfunction is causative of, or a consequence of, altered mitochondrial function including MPT.

General functional characteristics of the mitochondrial calicium uniporter such as cation selectivity, inhibitor and activator sensitivities, kinetic behavior, etc., have been reviewed (e.g., Gunter and Pfeiffer, 1990 Am. J Physiol. 258:C755-C786; Gunter et al., 1994 Am. J Physiol. 267:C313-C339). Properties of particular interest include the fact that Ca²⁺ flux into mitochondria via the uniporter increases as Δψ increases, at a given external concentration of the cation (Gunter et al., 1990; Gunter and Gunter, 1994 J. Bioenerg. Biomembr. 26:471-486). This behavior is analogous to the voltage dependence of ion flux through channels, as is the channel-like turnover number of the uniporter (˜20,000/sec) relative to carrier-type transporters, which typically exhibit turnover numbers approximately two orders of magnitude lower. Conversely, simple channels (e.g., gramicidin) show turnover numbers that are roughly two orders of magnitude higher than the mitochondrial calcium uniporter, consistent with a “gated channel” model for the uniporter (e.g., Litsky and Pfeiffer, 1997 Biochem. 36:7071-7078; Igbavboa et al., 1991 Biochim. Biophys. Acta 1059:339; Igbavboa et al., 1991 J. Biol. Chem. 266:4283; Igbavboa et al., 1988 J. Biol. Chem. 263:1405; cf, e.g., Yousef et al., 1989 Biochim. Biophys. Acta 984:281-288).

The mechanism of mitochondrial calcium uptake is poorly understood at the molecular level, however, and neither the gene(s) encoding the mitochondrial calcium uniporter, nor the protein(s) responsible for such mitochondrial calcium uptake (e.g., one or more mitochondrial calcium uniporter polypeptides) have been identified. Previous efforts to define the uniporter involved attempts to isolate mitochondrial calcium uniporter protein(s) that typically yielded molecules that differed functionally from the uniporter (e.g., Rosier et al., 1980 FEBS Lett. 109:99; Rosier et al., 1981 Arch Biochem. Biophys. 210:549; Dubinsky et al., 1979 in Membrane Bioenergetics, (Lee, C. P., Schatz, G. and Ernster, L., eds.), pp. 267-280, Addison-Wesley, Reading, Mass.), or that could not unambiguously be characterized as uniporter proteins due to the presence of contaminants (e.g., Sokolove et al., 1983 Arch. Biochem. Biophys. 221:404; Sandri et al., 1979 Biochim. Biophys. Acta 558:214; Saris et al., 1993 J. Bioenerg. Biomembr. 25:307; Mironova et al., 1994 J. Bioenerg. Biomembr. 26:231; Villa et al., 1994 EBEC Short Rep. 8:84; Zazueta et al., 1994 J. Bioenerg. Biomembr. 26:555).

The structural identification of one or more polypeptides responsible for mitochondrial calcium uniporter activity, and the regulated nucleic acid sequences (e.g., genes) that encode and express such polypeptide(s), would facilitate research into mitochondrial calcium regulation, as well as provide a target for therapeutic agents to prevent or treat diseases associated with altered (e.g., increased or decreased in a statistically significant manner) mitochondrial function. Thus, in view of the significance of mitochondrially regulated intracellular calcium levels and their relationship to several disease states, there is clearly a need for improved compositions and methods that may be used to identify a mitochondrial divalent cation transporter such as the calcium uniporter. Moreover, agents that alter (e.g., increase or decrease in a statistically significant manner) mitochondrial calcium regulation may be beneficial, and assays to specifically detect such agents are needed. The present invention fulfills these needs and further provides other related advantages.

### SUMMARY OF THE INVENTION

The present invention is generally directed to nucleic acid expression constructs encoding mitochondrial calcium uniporter polypeptide(s); to functional screening methods for detecting such polypeptide(s) expressed in suitable host cells; and to identification of mitochondrial calcium uniporter polypeptide(s). Accordingly, it is an aspect of the invention to provide a method for identifying a nucleic acid molecule encoding a mitochondrial divalent cation transporter polypeptide, comprising: a) contacting a biological sample comprising a host cell comprising at least one mitochondrion with at least one nucleic acid expression construct under conditions and for a time sufficient to permit expression of at least one mitochondrial divalent cation transporter polypeptide, wherein (i) the mitochondrion comprises a divalent cation-sensitive indicator molecule that is capable of generating a detectable signal in the presence of a divalent cation, and (ii) said nucleic acid expression construct comprises a promoter operably linked to a nucleic acid encoding a candidate mitochondrial divalent cation transporter polypeptide; b) exposing the host cell to a divalent cation under conditions and for a time sufficient to permit transport of the divalent cation across a membrane by the candidate mitochondrial divalent cation transporter; and c) detecting a signal generated by the divalent cation-sensitive indicator molecule in at least one mitochondrion, and therefrom identifying a nucleic acid encoding a mitochondrial divalent cation transporter polypeptide. In certain embodiments the divalent cation is barium, calcium, cobalt, iron, a lanthanide series member, lead, magnesium, manganese, zinc or strontium, and in certain embodiments the divalent cation is calcium. In certain embodiments the host cell is a prokaryotic cell and in certain embodiments the host cell is a eukaryotic cell, which in certain further embodiments is a yeast cell, and in certain still further embodiments is Saccharomyces cerevisiae, Schizosacchromyces pombe, Candida albicans or Pichia pastoris. In some embodiments the host cell mitochondrion lacks an endogenous electrogenic divalent cation transporter. In certain embodiments activity of at least one endogenous gene product is substantially impaired, wherein the gene product is an electrogenic divalent cation transporter or an electroneutral divalent cation transporter. In certain embodiments the nucleic acid expression construct further comprises at least one additional polynucleotide that regulates transcription. In certain further embodiments the additional polynucleotide that regulates transcription encodes a repressor of said regulated promoter.

In certain other embodiments the nucleic acid expression construct encodes a candidate mitochondrial divalent cation transporter polypeptide that is expressed as a fusion protein with a polypeptide product of a second polynucleotide. In certain embodiments the fusion protein localizes to a cellular membrane, which in certain further embodiments is a mitochondrial membrane, a vacuolar membrane, a vesicular membrane, an endoplasmic reticulum membrane, a Golgi membrane, a chloroplast membrane or a plasma membrane. In certain embodiments the cellular membrane is a mitochondrial membrane, which in certain further embodiments is an inner mitochondrial membrane. In certain embodiments the divalent cation-sensitive indicator molecule is an aequorin protein, luciferase, a green fluorescent protein or variant thereof, ⁴⁵Ca, Rhod-2, fura-2, Indo-1, Fluo-3 or a FLASH sequence.

According to certain embodiments of the above described method for identifying a nucleic acid molecule encoding a mitochondrial divalent cation transporter polypeptide the host cell mitochondrion comprises at least one esterase that is capable of cleaving a divalent cation-sensitive indicator molecule precursor to provide the divalent cation-sensitive indicator molecule. In other embodiments the host cell comprises at least one second nucleic acid expression construct which directs expression of an esterase that localizes to a mitochondrion, wherein the esterase is capable of cleaving a divalent cation-sensitive indicator molecule precursor to provide the divalent cation-sensitive indicator molecule. In certain further embodiments the divalent cation-sensitive indicator molecule precursor is capable of crossing a cellular membrane, which in certain embodiments may be a mitochondrial membrane, a vacuolar membrane, a vesicular membrane, an endoplasmic reticulum membrane, a Golgi membrane, a chloroplast membrane or a plasma membrane. In certain other further embodiments the esterase comprises a mitochondrial targeting sequence. In certain other further embodiments the divalent cation-sensitive indicator molecule precursor is an ester of a divalent cation-sensitive indicator molecule that is Indo-1 or Fura-2.

In certain embodiments the mitochondrial divalent cation transporter polypeptide comprises a divalent cation uniporter polypeptide, and in certain embodiments the mitochondrial divalent cation transporter polypeptide comprises an electrogenic divalent cation transporter polypeptide. In certain further embodiments the divalent cation uniporter is a calcium uniporter. In another embodiment the signal generated by the divalent cation-sensitive indicator molecule is detectable by spectrophotometry, radiometry, fluorimetry, FRET or flow cytofluorimetry.

In another embodiment, the invention provides a method of identifying a nucleic acid encoding a mitochondrial divalent cation transporter polypeptide, comprising: a) contacting a host cell with at least one nucleic acid expression construct under conditions and for a time sufficient to permit expression of at least one mitochondrial divalent cation transporter polypeptide, wherein (i) host cell growth is impaired in the presence of Ca2+, and (ii) said nucleic acid expression construct comprises a promoter operably linked to a nucleic acid encoding a candidate mitochondrial divalent cation transporter polypeptide; b) exposing the host cell to a divalent cation under conditions and for a time sufficient to permit transport of the divalent cation across a membrane by the candidate mitochondrial divalent cation transporter; and c) detecting cell growth in at least one host cell, and therefrom identifying a nucleic acid encoding a mitochondrial divalent cation transporter polypeptide. In certain embodiments the host cell is a eukaryotic cell, and in certain other embodiments the host cell is a prokaryotic cell. In certain embodiments the eukaryotic host cell is a yeast cell that is Sacchromyces cerevisiae, Sacchromyces pombe, Candida albicans or Pichia pastoris. In certain other embodiments the host cell comprises a mutated ATPase gene, while in certain other embodiments the host cell comprises a vacuolar assembly mutation. In certain embodiments the host cell comprises a yeast PMC1/PMR1 double ATPase mutant. In certain other embodiments the mitochondrial divalent cation transporter polypeptide comprises a uniporter, which in certain further embodiments comprises a calcium uniporter. In certain embodiments the divalent cation is calcium, cobalt, iron, lead, a member of the lanthanide series, magnesium, manganese, zinc or strontium. In certain embodiments the divalent cation is calcium that is exposed to the host cell at a concentration from about 0.01 μM to about 100 μM. In another embodiment cell growth is detected by microscopy, enzyme activity, spectrophotometry, flow cytometry, fluorimetry, or luminometry.

In another embodiment, the invention provides a method of preparing a mitochondrial divalent cation transporter polypeptide, comprising culturing a host cell comprising a nucleic acid expression construct that encodes a protein comprising a candidate mitochondrial divalent cation transporter polypeptide identified according to the above described methods, under conditions and for a time sufficient to permit expression of the polypeptide, and recovering the polypeptide. In certain embodiments the host cell is a prokaryotic cell, and in certain other embodiments the host cell is a eukaryotic cell, which in certain further embodiments is a yeast cell that is Sacchromyces cerevisiae, Schizosacchromyces pombe, Candida albicans or Pichia pastoris. In certain embodiments the mitochondrial divalent cation transporter polypeptide comprises a uniporter, which in certain further embodiments comprises a calcium uniporter.

These and other aspects of the present invention will become apparent upon reference to the following detailed description and attached drawings. All references disclosed herein are hereby incorporated by reference in their entireties as if each was incorporated individually.

### BRIEF DESCRIPTION OF THE DRAWINGS

FIG. 1 shows the bond-line structure of ionophore ETH 129. Cation liganding atoms are marked with an asterisk.

FIG. 2 shows conditions effecting ETH 129 mediated Ca2+ transport.

FIG. 3 shows the effect of membrane potential on the rate of transport.

FIG. 4 shows the effect of pH conditions on the rate of Ca²⁺ transport.

FIG. 5 shows the effect of CCP concentration on the rate of calcium cation transport.

FIG. 6 shows the effect of ionophore (ETH 129) concentration on the rate of Ca²+ transport.

FIG. 7 shows the effect of Ca2+concentration on the rate of Ca2+ transport.

FIG. 8 shows the cation specificity of ETH 129 facilitated transport.

FIG. 9 shows variable efficiency of ETH 129 mediated Ca2+ transport in yeast mitochondria.

FIG. 10 shows Ca2+ accumulation by freshly prepared yeast mitochondria.

FIG. 11 shows the effects of ETH 129 and Ca2+ on ΔΨ.

FIG. 12 shows restoration of AT in the presence of ETH 129 and Ca2+.

FIG. 13 shows the effects of ETH 129 and Ca2+ on mitochondrial swelling and respiration.

FIG. 14 shows the effect of exogenous oleate on ETH 129-mediated Ca2+ transport.

FIG. 15 restoration of ΔΨ by EGTA in the presence of ETH 129 and Ca2+.

FIG. 16 shows differential effects of oleate on AT in the presence and absence of EGTA.

FIG. 17 shows phospholipase activity in isolated yeast mitochondria.

FIG. 18 shows calibration data obtained with lysed mitochondria.

FIG. 19 shows aequorin luminescence in intact yeast cells.

FIG. 20 shows ionophore dependent active accumulation and retention of Ca²⁺ by yeast mitochondria.

FIG. 21 shows luminescence properties of isolated yeast mitochondria.

FIG. 22 shows accumulation and release of Ca²⁺ in yeast mitochondria.

FIG. 23 shows mitochondrial equilibration of medium free Ca²⁺ and matrix free Ca²⁺ concentrations.

FIG. 24 shows the rate of change in the mitochondrial matrix Ca²⁺ concentration as a function of the external concentration.

FIG. 25 shows Ca²⁺ release from previously loaded yeast mitochondria.

FIG. 26 shows growth inhibition by Ca²+ of Saccharomyces cerevisiae strain K473.

FIG. 27 presents a growth curve showing growth inhibition by Ca²+ of Saccharomyces cerevisiae strain K473.

### DETAILED DESCRIPTION OF THE INVENTION

The present invention provides methods for use in identifying nucleic acid sequences encoding one or more polypeptides that participate in transporting small molecules between subcellular compartments. In particular, the invention is directed in pertinent part to a method for identifying a nucleic acid molecule encoding a mitochondrial divalent cation transporter polypeptide, for instance, a mitochondrial calcium uniporter. The invention is, therefore, generally directed to the unexpected discovery of compositions and methods that are useful for expression-screening of nucleic acid libraries in particular host cells and under particular conditions as described herein, to survey for expression of a mitochondrial divalent cation transporter polypeptide, for example, a mitochondrial calcium uniporter.

Thus, in certain embodiments the present invention contemplates identifying a divalent cation transporter polypeptide by exploiting a host cell that comprises at least one esterase that is capable of cleaving a divalent cation-sensitive indicator molecule precursor to provide a divalent cation-sensitive indicator molecule, such as a host cell comprising a mitochondrially targeted esterase or a host cell comprising a nucleic acid constuct which directs expression of a mitochondrially targeted esterase. In certain embodiments the present invention contemplates identifying a divalent cation transporter polypeptide by exploiting a host cell that lacks, or that is substantially impaired with regard to, expression of one or more particular divalent cation transport and/or related activities. In certain embodiments organellar cation transport may be absent or substantially impaired in such a host cell as provided herein, which in certain preferred embodiments relates to a host cell that lacks endogenous mitochondrial calcium uniporter activity, and which in certain other preferred embodiments relates to a host cell that has one or more defects in organellar membrane calcium transport, such as calcium transport in non-mitochondrial organellar membranes including, for example, Golgi, endoplasmic reticulum, vacuolar and/or other cellular membranes.

Accordingly, and as described in greater detail below, the present invention relates in pertinent part to identification and construction of a host cell in which a nucleic acid expression construct, for example a plurality of such constructs comprising nucleic acids encoding candidate mitochondrial divalent cation transporter polypeptides and generated from a nucleic acid library, may be expressed in a manner that permits detection of the expressed product. The invention relates further, inter alia, to identification and construction of such a host cell comprising a mitochondrion that comprises a divalent cation-sensitive indicator molecule, and to compositions and methods for use with such a cell, in order to identify a nucleic acid molecule encoding a mitochondrial divalent cation transporter polypeptide such as a calcium uniporter.

For example, and as described in greater detail below, the invention relates in certain embodiments to the exploitation of cells from certain yeast strains, such as strains of Saccharomyces cerevisiae that lack endogenous mitochondrial calcium uniporter activity. These cells further comprise a divalent cation-sensitive indicator molecule, such as the product of esterase cleavage (e.g., hydrolysis of an ester linkage) of a divalent cation-sensitive indicator molecule precursor to provide a divalent cation-sensitive indicator molecule as provided herein; such cells may also comprise a nucleic acid expression construct which directs expression of such an esterase. For instance, yeast cells may be transfected with an esterase encoding gene under conditions and for a time sufficient to permit one or more mitochondria within such cells to accumulate a detectable level of one or more divalent cation-sensitive indicator molecules that may be low molecular weight or small molecules, and/or that are not proteins. As another example, yeast cells that may be useful according to the present invention may comprise a divalent cation-sensitive indicator molecule that is the product of a productively transfected photoprotein-encoding gene (e.g., aequorin) and, optionally, an added photoprotein cofactor (e.g., coelenterazine). Transfection of such cells, or of essentially similar cells, with expression constructs containing coding sequences for candidate calcium uniporter polypeptides, under conditions permissive for expression of such transgenes, provides a cell population that can be screened for the appearance of calcium uniporter activity, i.e., translocation of extramitochondrial calcium cations into the mitochondria, as detected by a signal generated by the mitochondrial cation-sensitive indicator molecule. Assay conditions and agents for determining the presence of calcium uniporter activity, as distinguished from mitochondrial calcium uptake by other transport mechanisms such as transmembrane cation exchanger proteins, are also described herein.

For instance, characterization of the divalent cation ionophore ETH 129 as an electrogenic ionophore as provided herein, and determination through the use of ETH 129 of conditions whereby yeast mitochondria can predictably load or release Ca2+, are described below. As another example, in certain embodiments of the present invention there are also provided strategies whereby defective calcium-dependent components of yeast secretory pathways may be exploited to devise a cell-based system in which calcium cations may act as a negative selective agent, resistance to which is conferred by transfection-mediated restoration of a divalent cation transporter polypeptide such as a mitochondrial calcium uniporter polypeptide.

Divalent Cation Transporter Polypeptide(S)

The present invention is directed generally to the identification of mitochondrial divalent cation transporter polypeptide(s). Some divalent cations are recognized as intracellular messengers that are maintained at low cytoplasmic levels. For example, as noted above, excess levels of cytoplasmic Ca²⁺ may lead to either necrotic or apoptotic forms of cell death in certain cells. The concentration of free Ca²⁺ in the cell cytoplasm (e.g., ≦10 μM) is normally about a thousand fold lower than the concentration of extracellular Ca²⁺ (e.g., ≦1 mM) because Ca²⁺ entry is closely regulated at the plasma membrane and because most of the Ca²⁺ in cells is bound to other molecules or sequestered in mitochondria and other intracellular organelles. Maintenance of intracellular calcium homeostasis is thus performed at least in part by certain specialized cellular membrane-associated polypeptides that mediate passage of calcium (e.g., as Ca²+ cations) across biological membranes, and that are included in the class of biological molecules referred to generally as membrane transport proteins. As used herein, “membrane transport protein” may be used interchangeably with “transport protein,” “transporter polypeptide,” or “transporter.” In certain preferred embodiments, the methods of the present invention are directed to identifying a nucleic acid molecule encoding a divalent cation transporter that mediates cation transfer across the inner mitochondrial membrane.

Thus, a divalent cation transporter as provided herein is, in preferred embodiments, the mitochondrial calcium uniporter described above. As provided herein, the mitochondrial calcium uniporter is an electrogenic calcium transporter. By way of background, ion transporters may be classified on the basis of whether they facilitate transmembrane movement of an ion by a mechanism that is electroneutral, or by a mechanism that is electrogenic. Electroneutral transporters do not carry a net charge, and mediate transmembrane delivery of one ionic species by exchanging such ion for a charge equivalent (e.g., Ca²⁺ is exchanged for 2H⁺) such that no net charge movement across the membrane accompanies transport of the ion(s). Transport catalyzed by electroneutral ionophores, for example, can be influenced by transmembrane pH gradients. Electrogenic transporters, on the other hand, contain no ionizable functions and form complexes with the ionic species being transported that are consequently charged, such that transmembrane charge movement does accompany transport of the ion (see, e.g., Dobler, 1981, Classification of Ionophores, In Ionophores and Their Structures, John Wiley and Sons, New York, NY; Westley, 1982, Notation and Classification, In Polyether Antibiotics: Naturally Occurring Acid Ionophores (Westley, J. W., editor), Marcel Dekker, New York, N.Y.). Membrane potential may have a significant influence on electrogenic transporters (Wooley et al., 1995, The Use of Ionophores for Manipulating Intracellular Ion Concentrations, In Methods in Neurosciences (Krajcer, J. and Dixon, S. J., eds.), Academic Press, Orlando, Fla.). In preferred embodiments of the present invention, a transporter polypeptide is a mitochondrial divalent cation transport polypeptide, and in certain particularly preferred embodiments the mitochondrial divalent cation transport polypeptide is a component of a calcium uniporter or other electrogenic transporter. Those having ordinary skill in the art will appreciate that divalent cations (i.e., chemical or biochemical species having a net charge of +2) that may be delivered across a biological membrane include, but need not be limited, to calcium, cobalt, barium, iron, members of the lanthanide series including lanthanum, lead, magnesium, manganese, zinc and strontium.

Nucleic Acids and Expression Constructs

The polynucleotides, or nucleic acid molecules, of the present invention may be comprised of a wide variety of nucleotides, including DNA, RNA, nucleotide analogues such as phosphorothioates or peptide nucleic acids, or other analogues with which those skilled in the art will be familiar, or some combination thereof. The nucleic acid constructs may be in the form of RNA or in the form of DNA; the DNA may include genomic DNA, cDNA, and synthetic DNA. The DNA may be double-stranded or single-stranded, and if single stranded may be the coding strand or non-coding (anti-sense) strand.

The nucleic acids which encode mitochondrial divalent cation transport polypeptides, for example a mammalian mitochondrial calcium uniporter or any other mitochondrial divalent cation transport polypeptides for use according to the invention, may include, but are not limited to: only the coding sequence for the mitochondrial divalent cation transport polypeptide; the coding sequence for the mitochondrial divalent cation transport polypeptide and at least one additional coding sequence; or, the coding sequence for the mitochondrial divalent cation transport polypeptide (and optionally additional coding sequence) and at least one non-coding sequence. Similarly, the nucleic acids which encode an esterase that is capable of cleaving a divalent cation-sensitive indicator molecule precursor, as provided herein, may include, but are not limited to: only the coding sequence for the esterase; the coding sequence for the esterase and at least one additional coding sequence (e.g., a mitochondrial targeting sequence); or, the coding sequence for the esterase (and optionally additional coding sequence) and at least one non-coding sequence.

A non-coding sequence may include, for example, one or more of introns or non-coding sequences situated 5′ and/or 3′ to the coding sequence for the mitochondrial divalent cation transport polypeptide. Coding or non-coding nucleic acids may, for example, further include (but need not be limited to) one or more regulatory nucleic acid sequences that may be a regulated or regulatable promoter, enhancer, other transcription regulatory sequence, repressor binding sequence, translation regulatory sequence or any other regulatory nucleic acid sequence. Thus, the term “nucleic acid encoding a mitochondrial divalent cation transport polypeptide” encompasses a nucleic acid which includes only coding sequence for the transport polypeptide as well as a nucleic acid which includes additional coding and/or non-coding sequence(s), and the term “nucleic acid encoding an esterase that is capable of cleaving a divalent cation-sensitive indicator molecule precursor” encompasses a nucleic acid which includes only coding sequence for the esterase polypeptide as well as a nucleic acid which includes additional coding and/or non-coding sequence(s).

Accordingly, as described herein and as will be known to those familiar with the art, disclosure below as may relate generally to nucleic acids, nucleic acid expression constructs and vectors, although in certain instances presented in a manner pertaining to nucleic acids encoding, for example, a mitochondrial divalent cation transport polypeptide, may also be regarded as pertaining to nucleic acids encoding, for example, an esterase that is capable of cleaving a divalent cation-sensitive indicator molecule precursor to provide a divalent cation-sensitive indicator molecule, to the extent that general features and properties of such nucleic acids (i.e., properties that are independent of the encoded polypeptide of interest such as the transporter or esterase polypeptides) are described. Such general disclosures herein may also relate to nucleic acid variants, fragments, analogs and derivatives, to nucleic acid hybridization and to equivalent DNA constructs, as provided herein, and are therefore intended to include nucleic acid compositions and methods applicable to nucleic acids encoding any polypeptide of interest (e.g., a cation transporter, an esterase, a mitochondrial targeting sequence, a fusion protein domain, etc.).

The present invention further relates to variants of the herein described nucleic acids which encode for fragments, analogs and derivatives of a mitochondrial divalent cation transport polypeptide. The variants of the nucleic acids encoding mitochondrial divalent cation transport polypeptides may be naturally occurring allelic variants of the nucleic acids or non-naturally occurring variants. As is known in the art, an allelic variant is an alternate form of a nucleic acid sequence which may have at least one of a substitution, a deletion or an addition of one or more nucleotides, any of which does not substantially alter the function of the encoded mitochondrial divalent cation transport polypeptide. Thus, for example, the present invention includes nucleic acids encoding a mammalian mitochondrial calcium uniporter polypeptide, as well as variants of such nucleic acids, which variants encode a fragment, derivative or analog of any such polypeptide.

Accordingly, the present invention further relates to nucleic acids which hybridize to transporter polypeptide encoding sequences as provided herein, as incorporated by reference or as will be readily apparent to those familiar with the art, if there is at least 70%, preferably at least 90%, and more preferably at least 95% identity between the sequences. The present invention particularly relates to nucleic acids which hybridize under stringent conditions to the transporter polypeptide encoding sequences referred to herein. As used herein, the term “stringent conditions” means hybridization will occur only if there is at least 95% and preferably at least 97% identity between the sequences. In preferred embodiments, the nucleic acids which hybridize to transporter polypeptide encoding sequences referred to herein, or their complements, encode polypeptides which retain substantially the same biological function or activity as the transporter polypeptide encoded by the identified nucleic acid sequences.

As used herein, to “hybridize” under conditions of a specified stringency is used to describe the stability of hybrids formed between two single-stranded nucleic acid molecules. Stringency of hybridization is typically expressed in conditions of ionic strength and temperature at which such hybrids are annealed and washed. Typically “high”, “medium” and “low” stringency encompass the following conditions or equivalent conditions thereto: high stringency: 0.1× SSPE or SSC, 0.1% SDS, 65° C.; medium stringency: 0.2× SSPE or SSC, 0.1% SDS, 50° C.; and low stringency: 1.0× SSPE or SSC, 0.1% SDS, 50° C. As known to those having ordinary skill in the art, variations in stringency of hybridization conditions may be achieved by altering the time, temperature and/or concentration of the solutions used for prehybridization, hybridization and wash steps, and suitable conditions may also depend in part on the particular nucleotide sequences of the probe used, and of the blotted, proband nucleic acid sample. Accordingly, it will be appreciated that suitably stringent conditions can be readily selected without undue experimentation where a desired selectivity of the probe is identified, based on its ability to hybridize to one or more certain proband sequences while not hybridizing to certain other proband sequences.

Variants and derivatives of any divalent cation transport polypeptide, including mitochondrial divalent cation transporter polypeptides identified according to the methods of the present invention, may be obtained by mutations (e.g, of nucleotide sequences such as those identified according to the disclosure herein) that encode such polypeptides. Similarly, variants and derivatives of esterase polypeptides for use according to certain embodiments of the present invention may be obtained by mutations of coding sequences, for example, as disclosed in U.S. Pat. Nos. 5,741,961, 5,906,930 and 5,945,325, all of which are incorporated by reference. Alterations of the native amino acid sequence may be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene wherein predetermined codons can be altered by substitution, deletion or insertion. Exemplary methods of making such alterations are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462.

Equivalent DNA constructs that encode various additions or substitutions of amino acid residues or sequences, or deletions of terminal or internal residues or sequences not needed for biological activity are also encompassed by the invention. For example, sequences encoding Cys residues that are not essential for biological activity can be altered to cause the Cys residues to be deleted or replaced with other amino acids, preventing formation of incorrect intramolecular disulfide bridges upon renaturation. Other equivalents can be prepared by modification of adjacent dibasic amino acid residues to enhance expression in yeast systems in which KEX2 protease activity is present. EP 212,914 discloses the use of site-specific mutagenesis to inactivate KEX2 protease processing sites in a protein. KEX2 protease processing sites are inactivated by deleting, adding or substituting residues to alter Arg-Arg, Arg-Lys, and Lys-Arg pairs to eliminate the occurrence of these adjacent basic residues. Lys-Lys pairings are considerably less susceptible to KEX2 cleavage, and conversion of Arg-Lys or Lys-Arg to Lys-Lys represents a conservative and preferred approach to inactivating KEX2 sites.

An “isolated nucleic acid molecule” refers to a polynucleotide molecule, for example, in the form of a separate fragment or as a component of a larger nucleic acid construct, that has been removed from its original environment (e.g., its natural source cell) (including the chromosome it normally resides in) at least once, and in certain embodiments, in a substantially pure form. Isolated nucleic acids may include one or more fragments of unknown nucleotide sequence(s) or nucleic acids having particular disclosed nucleotide sequences, and/or regions, portions or fragments thereof. For example, a population of isolated nucleic acids of unknown nucleotide sequences may together represent a complete chromosomal genome, mitochondrial genome, or cDNA population. Those having ordinary skill in the art are able to prepare isolated nucleic acids having unknown nucleotide sequences and, when provided with the appropriate nucleic acid sequence information, are also able to prepare a complete nucleotide sequence or the sequence of any portion of a particular isolated nucleic acid molecule.

The present invention, as described herein, provides a method for identifying a nucleic acid molecule encoding a mitochondrial divalent cation transporter polypeptide(s) that relates, in pertinent part, to expression of a nucleic acid expression construct as provided herein that comprises a promoter operably linked to a nucleic acid encoding a candidate mitochondrial divalent cation transporter. An appropriate genetic library of candidate nucleic acids encoding such a candidate mitochondrial divalent cation transporter may be constructed according to techniques well known in the art (see, e.g., Sambrook et al., Molecular Cloning: A Laboratory Manual, Cold Spring Harbor Press, 1989), or a similar library may be purchased from commercial sources (e.g., Clontech, Palo Alto, Calif.). According to the methods described herein, a nucleic acid molecule encoding a mitochondrial divalent cation transporter polypeptide (e.g., the mitochondrial calcium uniporter) may be identified from within a heterogeneous population of polynucleotide fragments derived from such a library.

Typically, genetic libraries may be constructed by combining a desired vector with a collection or set of nucleic acids (such as genomic DNA or cDNA and sometimes synthetic DNA or RNA), which together represent all or some portion of a genome, a population of mRNAs, or some other set of nucleic acid molecules that contain sequences of interest. Preferably, a genetic library of nucleic acid expression constructs is Generated by combining a suitable vector with genomic DNA to generate nucleic acid expression constructs, more preferably a vector is combined with CDNA synthesized from total RNA (e.g., by using reverse transcriptase), and most preferably a vector is combined with CDNA synthesized from mRNA, which includes sequences encoding candidate mitochondrial divalent cation transporter polypeptide(s).

More particularly, the present invention relates to “nucleic acid expression constructs” that include any polynucleotides (e.g., nucleic acid molecules) encoding candidate mitochondrial divalent cation transporter polypeptide(s), such as a mitochondrial calcium uniporter as described herein; to host cells that are genetically engineered with vectors and/or constructs of the invention and to the identification of transporter polypeptides by functional assays as described herein. Accordingly, a library of nucleic acid expression constructs may comprise a promoter operably linked to a polynucleotide encoding, for example, a candidate mitochondrial calcium uniporter polypeptide, fragment, or derivative thereof. Candidate mitochondrial divalent cation transporter polypeptide(s) may be expressed in eukaryotic cells (e.g., mammalian, plant, or yeast cells), or prokaryotic cells (e.g., bacteria) under the control of appropriate promoters. Cell-free translation systems may also be employed to produce such proteins using RNAs derived from the DNA constructs of the present invention. Appropriate cloning and expression vectors for use with prokaryotic and eukaryotic hosts are described, for example, by Sambrook, et al., Molecular Cloning: A Laboratory Manual, Second Edition, Cold Spring Harbor, N.Y., (1989), and may include plasmids, cosmids, shuttle vectors, viral vectors (e.g., bacteriophage or retroviral) and vectors comprising a chromosomal origin of replication (such as yeast artificial chromosomes) as disclosed therein.

Generally, nucleic acid expression vectors include origins of replication and selectable markers permitting detectable transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), co-factor, acid phosphatase, or heat shock proteins, among others. The heterologous nucleic acid sequences expressed in a host cell are generally those that insert into the nucleic acid expression vector in the appropriate phase with translation initiation and termination sequences. Optionally, the heterologous sequence may encode a fusion protein including an N-terminal (or a C-terminal) identification peptide imparting desired characteristics, e.g., stabilization or simplified purification of expressed recombinant product.

Expression constructs for bacterial use may be constructed, for example, by randomly inserting into an expression vector a population of cDNA molecules that include a polynucleotide sequence encoding candidate mitochondrial divalent cation transporter polypeptide(s) together with suitable translation initiation and termination signals in operable reading phase with a functional promoter. The construct may comprise one or more phenotypic selectable markers and an origin of replication to ensure maintenance of the vector construct and, if desired, to provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice. Any other plasmid or vector may be used as long as they are replicable and viable in the host.

Polypeptides and Fusion Proteins

The present invention further relates to mitochondrial divalent cation transporter polypeptides, including methods for producing recombinant mitochondrial divalent cation transporter polypeptides by culturing host cells containing mitochondrial divalent cation transporter expression constructs, and to isolated recombinant mitochondrial divalent cation transporter polypeptides, including, for example, a mitochondrial calcium uniporter polypeptide as provided herein. The polypeptides and nucleic acids of the present invention are preferably provided in an isolated form, and in certain preferred embodiments are purified to homogeneity.

The terms “fragment,” “derivative” and “analog” when referring to mitochondrial divalent cation transporter polypeptides or fusion proteins refers to any mitochondrial divalent cation transporter polypeptide or fusion protein that retains essentially the same biological function or activity as such polypeptide. Similarly, fragments, derivatives or analogs of esterases capable of cleaving a divalent cation-sensitive indicator molecule precursor to provide a divalent cation-sensitive indicator molecule, as provided herein, refers to any esterase polypeptide or fusion protein that retains essentially the same (or in certain instances, superior) enzyme catalytic function or activity as such polypeptide. Thus, an analog includes a proprotein which can be activated by cleavage of the proprotein portion to produce an active mitochondrial divalent cation transporter polypeptide, or to produce an esterase polypeptide. The polypeptide of the present invention may be a recombinant polypeptide or a synthetic polypeptide, and is preferably a recombinant polypeptide.

A fragment, derivative or analog of a mitochondrial divalent cation transporter (or esterase) polypeptide or fusion protein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mitochondrial divalent cation transporter (or esterase) polypeptide is fused with another compound, such as a compound to increase the half-life of the polypeptide (for example, polyethylene glycol), or (iv) one in which additional amino acids are fused to the mitochondrial divalent cation transporter (or esterase) polypeptide, including amino acids that are employed for purification of the mitochondrial divalent cation transporter (or esterase) polypeptide or a proprotein sequence. Such fragments, derivatives and analogs are deemed to be within the scope of those skilled in the art from the teachings herein.

The polypeptides of the present invention include mitochondrial divalent cation transporter polypeptides and fusion proteins, and esterase polypeptides and fusion proteins, having amino acid sequences that are identical or similar to sequences encoded by nucleic acid expression constructs identified according to the methods provided herein. As known in the art “similarity” between two polypeptides is determined by comparing the amino acid sequence and conserved amino acid substitutions thereto of a polypeptide, to the sequence of a second polypeptide. Fragments or portions of the polypeptides of the present invention may be employed for producing the corresponding full-length polypeptide by peptide synthesis; therefore, the fragments may be employed as intermediates for producing the full-length polypeptides. Fragments or portions of the nucleic acids of the present invention may be used to synthesize full-length nucleic acids of the present invention.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acids could be part of a vector and/or such nucleic acids or polypeptides could be part of a composition, and still be isolated in that such vector or composition is not part of its natural environment.

The term “gene” means the segment of DNA involved in producing a polypeptide chain; it includes regions preceding and following the coding region “leader and trailer” as well as intervening sequences (introns) between individual coding segments (exons).

As described herein, the invention provides mitochondrial divalent cation transporter (and, in certain embodiments, esterase) fusion proteins encoded by nucleic acids that have the mitochondrial divalent cation transporter (and, optionally, esterase) coding sequence fused in frame to an additional coding sequence. Thus, in certain embodiments the invention provides for expression of a mitochondrial divalent cation transporter (and/or esterase) polypeptide sequence fused to an additional functional or non-functional polypeptide sequence that permits, for example by way of illustration and not limitation, intracellular targeting, detection, isolation and/or purification of the mitochondrial divalent cation transporter (and/or esterase) fusion protein. Such mitochondrial divalent cation transporter (or esterase) fusion proteins may permit detection, isolation and/or purification of the fusion protein by protein-protein affinity, metal affinity or charge affinity-based polypeptide purification, or by specific protease cleavage of a fusion protein containing a fusion sequence that is cleavable by a protease, such that the polypeptide of interest (e.g., mitochondrial divalent cation transporter or esterase) polypeptide is separable from the fusion protein.

Mitochondrial divalent cation transporter (or esterase) fusion proteins may therefore comprise polypeptide sequences added to the mitochondrial divalent cation transporter (or esterase) to facilitate detection and isolation of the transporter. Such peptides include, for example, poly-His or the antigenic identification peptides described in U.S. Pat. No. 5,011,912 and in Hopp et al., (1988 Bio/Technology 6:1204), or the XPRESS™ epitope tag (Invitrogen, Carlsbad, Calif.), or the like. The affinity sequence may be a hexa-histidine tag as supplied, for example, by a pBAD/His (Invitrogen) or a pQE-9 vector to provide for purification of the mature polypeptide fused to the marker in the case of a bacterial host, or, for example, the affinity sequence may be a hemagglutinin (HA) tag when a mammalian host, e.g., COS-7 cells, is used. The HA tag corresponds to an antibody defined epitope derived from the influenza hemagglutinin protein (Wilson et al., Cell 37:767 (1984)).

Mitochondrial divalent cation transporter (or esterase) fusion proteins may further comprise immunoglobulin constant region polypeptides added to the mitochondrial divalent cation transporter to facilitate detection, isolation and/or localization of the transporter. The immunoglobulin constant region polypeptide preferably is fused to the C-terminus of a mitochondrial divalent cation transporter polypeptide. General preparation of fusion proteins comprising heterologous polypeptides fused to various portions of antibody-derived polypeptides (including the Fc domain) has been described, e.g., by Ashkenazi et al. (Proc. Nat. Acad. Sci. USA 88:10535, 1991) and Byrn et al. (Nature 344:677, 1990). A gene fusion encoding the transporter:Fc fusion protein is inserted into an appropriate expression vector. In certain embodiments of the invention, transporter:Fc fusion proteins may be allowed to assemble much like antibody molecules, whereupon interchain disulfide bonds form between Fc polypeptides, yielding dimeric mitochondrial divalent cation transporter fusion proteins.

Mitochondrial divalent cation transporter fusion proteins having specific binding affinities for pre-selected antigens by virtue of fusion polypeptides comprising immunoglobulin V-region domains encoded by DNA sequences linked in-frame to sequences encoding the transporter are also within the scope of the invention, including variants and fragments thereof as provided herein. General strategies for the construction of fusion proteins having immunoglobulin V-region fusion polypeptides are disclosed, for example, in EP 0318554; U.S. Pat. No. 5,132,405; U.S. Pat. No. 5,091,513; and U.S. Pat. No. 5,476,786.

The nucleic acid of the present invention may also encode a fusion protein comprising a mitochondrial divalent cation transporter polypeptide fused to other polypeptides having desirable affinity properties, for example an enzyme such as glutathione-S-transferase. As another example, mitochondrial divalent cation transporter fusion proteins may also comprise a transporter polypeptide fused to a Staphylococcus aureus protein A polypeptide; protein A encoding nucleic acids and their use in constructing fusion proteins having affinity for immunoglobulin constant regions are disclosed generally, for example, in U.S. Pat. No. 5,100,788. Other useful affinity polypetides for construction of cation transporter fusion proteins may include streptavidin fusion proteins, as disclosed, for example, in WO 89/03422; U.S. Pat. No. 5,489,528; U.S. Pat. No. 5,672,691; WO 93/24631; U.S. Pat. No. 5,168,049; U.S. Pat. No. 5,272,254 and elsewhere, and avidin fusion proteins (see, e.g., EP 511,747). As provided herein and in the cited references, mitochondrial divalent cation transporter polypeptide sequences may be fused to fusion polypeptide sequences that may be full length fusion polypeptides and that may alternatively be variants or fragments thereof.

The present invention also provides a method of targeting a polypeptide of interest to a membrane, and in particular embodiments to a cellular membrane, and in further preferred embodiments to a mitochondrial membrane. This aspect of the invention is based on the unexpected observation that certain recombinant expression constructs as provided herein, which constructs include a nucleic acid encoding a first polypeptide that is a mitochondrial divalent cation transporter polypeptide, and that is expressed as a fusion protein with a second polypeptide sequence, provide recombinant divalent cation transporter fusion proteins capable of preferentially localizing to cell membranes. In certain embodiments the cell membrane is a prokaryotic cell membrane such as a bacterial cell membrane, for example an E. coli membrane. In other embodiments the cell membrane is a eukaryotic cell membrane such as a yeast or a mammalian cell membrane, for example a membrane of any eukaryotic cell contemplated herein. In other embodiments the polypeptide of interest is a fusion protein comprising a first polypeptide that is an esterase that is capable of cleaving a divalent cation-sensitive indicator molecule precursor to provide a divalent cation-sensitive indicator molecule, as provided herein, and a second polypeptide that is a mitochondrial targeting sequence, as also provided herein.

A cell membrane as used herein may be any cellular membrane, and typically refers to membranes that are in contact with cytosolic components, including intracellular membrane bounded compartments such as mitochondrial inner and outer membranes as described above, and also intracellular vesicles, vacuoles, ER-Golgi constituents, chloroplasts, other organelles and the like, as well as the plasma membrane. In preferred embodiments, a mitochondrial divalent cation transporter fusion protein may be targeted to a mitochondrial membrane. In other preferred embodiments, for example, recombinant expression constructs according to the invention may encode divalent cation transporter fusion proteins that contain polypeptide sequences that direct the fusion protein to be retained in the cytosol, to reside in the lumen of the endoplasmic reticulum (ER), to be secreted from a cell via the classical ER-Golgi secretory pathway, to be incorporated into the plasma membrane, to associate with a specific cytoplasmic component including the cytoplasmic domain of a transmembrane cell surface receptor or to be directed to a particular subcellular location by any of a variety of known intracellular protein sorting mechanisms with which those skilled in the art will be familiar. Accordingly, these and related embodiments are encompassed by the instant compositions and methods directed to targeting a polypeptide of interest to a predefined intracellular, membrane or extracellular localization.

Vectors

The present invention also relates to vectors and to constructs that include nucleic acids of the present invention, and in particular to “recombinant expression constructs” as described above, and to the production of mitochondrial divalent cation transporter polypeptides and fusion proteins of the invention, or fragments or variants thereof, by recombinant techniques. Mitochondrial divalent cation transporter proteins can be expressed in mammalian cells, yeast, bacteria, or other cells under the control of appropriate promoters, or in cell-free translation systems.

As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis., USA). These pBR322 “backbone” sections are combined with an appropriate promoter and the structural sequence to be expressed. As another example, the expression vector pBAD/His (Invitrogen, Carlsbad, Calif.) may be used. This vector contains the following elements operably linked in a 5′ to 3′ orientation: the inducible, but tightly regulatable, araBAD promoter; optimized E. coli translation initiation signals; an amino terminal polyhistidine(6×His)-encoding sequence (also referred to as a “His-Tag”); an XPRESS™ epitope-encoding sequence; an enterokinase cleavage site which can be used to remove the preceding N-terminal amino acids following protein purification, if so desired; a multiple cloning site; and an in-frame termination codon Following transformation of a suitable host strain and growth of the host strain to an appropriate cell density, the selected promoter, if it is a regulated promoter as provided herein, is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents; such methods are well know to those skilled in the art.

Thus, for example, the nucleic acids of the invention as provided herein may be included in any one of a variety of expression vector constructs as a recombinant expression construct for expressing a mitochondrial divalent cation transporter polypeptide. Such vectors and constructs include chromosomal, nonchromosomal and synthetic DNA sequences, e.g., derivatives of SV40; bacterial plasmids; phage DNA; baculovirus (e.g., pBlueBacHis2, Invitrogen, Carlsbad, Calif.); yeast plasmids, for example pYES2, pYESTrp2 (Invitrogen, Carlsbad, Calif.), and pYPGE2, which comprises a TRP1 selectable marker and the strong PGK promoter upstream from a multiple cloning site (Brunelli et al., 1993 Yeast 9:1299-1308); yeast artificial chromosomes (YACs); vectors derived from combinations of plasmids and phage DNA; shuttle vectors derived from combinations of plasmids and viral DNA or combinations of prokaryotic and eukaryotic plasmids (e.g., pcDNA3.1); viral DNA, such as vaccinia, adenovirus, fowl pox virus, and pseudorabies. However, any other vector may be used for preparation of a nucleic acid expression construct as long as it is replicable and viable in the host cell of interest. Further, in some preferred embodiments, nucleic acid expression constructs containing, inter alia, the polynucleotide coding sequence for candidate mitochondrial divalent cation transporter polypeptide(s) and fusion proteins may remain extrachromosomal, and in other preferred embodiments the expression constructs may integrate into at least one host cell chromosome.

The appropriate nucleic acid molecules may be inserted into the vector by a variety of procedures. In general, a DNA sequence may be inserted into an appropriate restriction endonuclease site(s) by procedures known in the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described, for example, in Ausubel et al. (1993 Current Protocols in Molecular Biology, Greene Publ. Assoc. Inc. & John Wiley & Sons, Inc., Boston, Mass.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.); and elsewhere.

The coding sequence in the expression vector is operatively linked to at least one appropriate expression control sequences (e.g., a promoter, a regulated promoter and/or an externally regulated promoter) to direct mRNA synthesis. Representative examples of such expression control sequences include Gall/Gall O promoter, LTR, or SV40 promoter, the E. coli lac or trp, the phage lambda PL promoter and other promoters known to control expression of genes in prokaryotic or eukaryotic cells or their viruses. Promoter regions can be selected from any desired gene and used in vectors containing selectable markers, such as CAT (chloramphenicol transferase) or URA3. Two appropriate CAT vectors are pKK232-8 and pCM7, and an appropriate URA3 vector is pYES2. Particular named bacterial promoters include lacI, lacZ, T3, T7, gpt, lambda P_{R}, lambda P_{L} and trp. Eukaryotic promoters include CMV immediate early, such as is provided in the shuttle vectors pEYFP-C1 and pECFP-N1 (Clontech Laboratories Inc., Palo Alto, Calif.), HSV thymidine kinase, early and late SV40, Gal1/Gal10 from yeast, LTRs from retrovirus, and mouse metallothionein-I. The high-level expression, constitutive PGK (phosphoglycerate kinase), GPD (glyceraldehyde 3 phosphate dehydrogenase) and ADH1 (alcohol dehydrogenase) promoters may also be used (see, e.g., U.S. Pat. Nos. 5,104,795 and 4,865,989, and references cited therein). Selection of the appropriate vector and promoter is well within the level of ordinary skill in the art, and preparation of certain particularly preferred nucleic acid expression constructs comprising at least one promoter or regulated promoter operably linked to a polynucleotide encoding, inter alia, candidate mitochondrial divalent cation transporter polypeptide(s) is described herein.

In certain preferred embodiments the expression control sequence is a “regulated promoter” an “externally regulated promoter,” which includes functional promoter sequences having activity that may be altered (e.g., increased or decreased) by an additional element, agent, molecule, component, co-factor or the like. An externally regulated promoter may comprise, for example, a repressor binding site, an activator binding site or any other regulatory sequence that controls expression of a polynucleotide sequence as provided herein. In certain particularly preferred embodiments, the externally regulated promoter is a tightly regulated promoter that is specifically inducible and that permits little or no transcription of polynucleotide sequences under its control in the absence of an induction signal, as is known to those familiar with the art and described, for example, in Guzman et al. (J. Bacteriol., 1995, 177:4121), Carra et al. (EMBO J., 1993, 12:35), Mayer (Gene, 1995, 163:41), Haldimann et al. (J. Bacteriol., 1998, 180:1277), Lutz et al. (Nuc. Ac. Res., 1997, 25:1203), Allgood et al. (Curr. Opin. Biotechnol., 1997, 8:474) and Makrides (Microbiol. Rev., 1996, 60:512). In other preferred embodiments of the invention, an externally regulated promoter is present that is inducible but that may not be tightly regulated. In certain other preferred embodiments a promoter is present in the recombinant expression construct of the invention that is not a regulated promoter; such a promoter may include, for example, a constitutive promoter such as an insect polyhedrin promoter or a yeast phosphoglycerate kinase promoter (see, e.g., Giraud et al., 1998 J. Mol. Biol. 281:409). A nucleic acid expression construct may also contain a ribosome binding site for translation initiation and a transcription terminator. A vector may also include appropriate sequences for amplifying expression.

Transcription of a DNA sequence encoding a polypeptide of the present invention by higher eukaryotes may be increased by inserting an enhancer sequence into the vector. Enhancers are cis-acting elements of DNA, usually about from 10 to 300 bp that act on a promoter to increase its transcription. Examples including the SV40 enhancer on the late side of the replication origin bp 100 to 270, a cytomegalovirus early promoter enhancer, the polyoma enhancer on the late side of the replication origin, and adenovirus enhancers.

As noted above, in certain embodiments the vector may be a viral vector such as a retroviral vector. For example, retroviruses from which a retroviral plasmid vector may be derived include, but are not limited to, Moloney Murine Leukemia Virus, spleen necrosis virus, retroviruses such as Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus.

A viral vector generally includes one or more promoters. Suitable promoters include, but are not limited to, the retroviral LTR; the SV40 promoter; and the human cytomegalovirus (CMV) promoter described in Miller, et al., Biotechniques 7:980-990 (1989), or any other promoter (e.g., cellular promoters such as eukaryotic cellular promoters including, but not limited to, the histone, pol III, and β-actin promoters). Other viral promoters include, but are not limited to, adenovirus promoters, thymidine kinase (TK) promoters, and B19 parvovirus promoters. The selection of a suitable promoter will be apparent to those skilled in the art from the teachings contained herein, and may be from among either regulated promoters or promoters as described above.

The retroviral plasmid vector may be employed to transduce packaging cell lines to form producer cell lines. Examples of packaging cells which may be transfected include, but are not limited to, the PE501, PA317, ψ-2, ψ-AM, PA12, T19-14X, VT-19-17-H2, ψCRE, ψCRIP, GP+E-86, GP+envAm12, and DAN cell lines as described in Miller, Human Gene Therapy, 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

A producer cell line generates infectious retroviral vector particles that include, for example, a polynucleotide sequence(s) encoding a mitochondrial calcium uniporter polypeptide(s). Such retroviral vector particles then may be employed to transduce eukaryotic cells either in vitro or in vivo. The transduced eukaryotic cells generally express the polynucleotide sequence(s) encoding the mitochondrial calcium uniporter polypeptide(s). Eukaryotic cells that may be transduced include, but are not limited to, embryonic stem cells, embryonic carcinoma cells, as well as hematopoetic stem cells, hepatocytes, fibroblasts, myoblasts, keratinocytes, endothelial cells, and bronchial epithelial cells. In certain preferred embodiments, the recombinant retroviral vectors of the present invention may be used to transduce PMC1 or PMR1 single calcium/ATPase mutants, or PMC1/PMR1 double mutants (see Cunningham et al., 1994 J. Cell Biol. 124:351-363; see also Cunningham et al., 1996, Molec. Cell. Biology 16:2226-37; and references cited therein).

As another example of an embodiment of the invention in which a viral vector is used to prepare a library of nucleic acid expression constructs, host cells transduced by a population of recombinant viral constructs where at least one construct directs the expression of a transporter polypeptide or functional fragment thereof may produce viral particles containing expressed transporter polypeptides or functional fragment thereof that are derived from portions of a host cell membrane incorporated by the viral particles during viral budding. In another preferred embodiment, polynucleotide sequences may be cloned into a baculovirus shuttle vector to construct a genetic library, which is then recombined with a baculovirus to generate recombinant baculovirus expression constructs that are used to infect, for example, Sf9 host cells as described in Baculovirus Expression Protocols, Methods in Molecular Biology Vol. 39, Christopher D. Richardson, Editor, Human Press, Totowa, N.J., 1995; Piwnica-Worms, “Expression of Proteins in Insect Cells Using Baculoviral Vectors,” Section II in Chapter 16 in: Short Protocols in Molecular Biology, 2^{nd} Ed., Ausubel et al., eds., John Wiley & Sons, New York, N.Y., 1992, pages 16-32 to 16-48.

As an example without limitation, the present invention provides a method of identifying and isolating nucleic acid expression construct(s), from within a library of genomic or cDNA fragments, in a host cell encoding candidate mitochondrial divalent cation transporter polypeptide(s) or functional fragments thereof. Preferably, a suitable host cell is contacted with the nucleic acid expression constructs of the library to induce uptake, such as by transformation or transfection. The recombinant host cell is grown to an appropriate cell density, the selected promoter, if it is an externally regulated promoter, is induced by appropriate means (e.g., temperature shift or chemical induction) and cells are cultured for an additional period. Accordingly, the host cells that take up at least one nucleic acid expression construct having a promoter operably linked to a polynucleotide encoding a candidate mitochondrial divalent cation transporter polypeptide (e.g., calcium uniporter) may be identified using the functional assays of the present invention as described herein. For example, an identified host cell expressing a mitochondrial calcium uniporter polypeptide(s) may be used to over-produce and isolate the protein(s) by methods well known to those skilled in the art. Typically, cells are harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents.

As noted above, the nucleic acid encoding the mitochondrial calcium uniporter has not been identified. For example, by way of illustration and not limitation, a nucleic acid that encodes a candidate mitochondrial divalent cation transporter polypeptide (e.g., a calcium uniporter) to be identified according to the present invention may either be identical or different, due to the redundancy or degeneracy of the genetic code, to an orphan coding sequence of a particular genomic database that encodes the same candidate mitochondrial divalent cation transporter polypeptide. Thus, the present invention includes variants of the candidate polynucleotides, which may encode fragments, analogs or derivatives of native transporter polypeptide(s).

Samples and Host Cells

Typically, the invention relates in part to a method for identifying a nucleic acid molecule encoding a mitochondrial divalent cation transporter polypeptide, comprising contacting a nucleic acid expression construct as provided herein with a sample, which in preferred embodiments is a biological sample and in particularly preferred embodiments is a biological sample comprising a host cell containing one or more mitochondria. In other preferred embodiments the biological sample contains one or more cellular membranes, including the plasma membrane and intracellular membrane bounded compartments such as endosomes, lysosomes, peroxisomes, mitochondria, chloroplasts, endocytic and secretory vesicles, ER-Golgi constituents, organelles and the like. Biological samples may be provided by obtaining a blood sample, biopsy specimen, tissue explant, organ culture or any other biological, tissue or cell preparation from a subject or a biological source. The subject or biological source may be a human or non-human animal, a plant, a unicellular or a multicellular organism, a primary cell culture or culture adapted cell line including but not limited to genetically engineered cell lines that may contain chromosomally integrated or episomal recombinant nucleic acid sequences, immortalized or immortalizable cell lines, somatic cell hybrid or cytoplasmic hybrid “cybrid” cell lines, differentiated or differentiatable cell lines, transformed cell lines and the like. In certain preferred embodiments a biological sample may comprise a host cell comprising a mitochondrion that comprises at least one esterase that is capable of cleaving a divalent cation-sensitive indicator molecule precursor to provide a divalent cation-sensitive indicator molecule. In certain other preferred embodiments a biological sample may comprise a host cell comprising a nucleic acid expression construct which directs expression of at least one esterase that localizes to a mitochondrion and that is capable of cleaving a divalent cation-sensitive indicator molecule precursor to provide a divalent cation-sensitive indicator molecule.

In certain embodiments of the invention the biological sample may be an intact cell and in certain embodiments the biological sample may be all or a portion of a biological source, for example, a tissue or organ culture, an explant, a larval or embryonic or other developmentally discrete form of an organism, or the like. According to certain embodiments of the invention, it may be preferred to use intact cells, whereas in certain other embodiments, the use of permeabilized cells may be preferred. A permeabilized cell is a cell that has been treated in a manner that results in a partial or complete loss of plasma membrane selective permeability. For instance, it may be desirable to permeabilize a cell in a manner that permits calcium cations in the extracellular milieu to diffuse into permeabilized cells and contact mitochondria. Thus, in this instance, permeabilization serves as an alternative to the use of a calcium ionophore. As another example, certain divalent cation-sensitive indicator molecules, as provided herein, may penetrate the plasma membrane at a moderate rate, or to a limited degree, unless their entry into the cytosol is facilitated in some manner. Permeabilization of cells is one manner by which the cytosolic entry of such indicator molecules can be facilitated.

Those having ordinary skill in the art are familiar with methods for permeabilizing cells, for example by way of illustration and not limitation, through the use of surfactants, detergents, phospholipids, phospholipid binding proteins, enzymes, viral membrane fusion proteins and the like; by exposure to certain bacterial toxins, such as α-hemolysin; by contact with hemolysins such as saponin (which is also a nonionic detergent, as is digitonin); through the use of osmotically active agents; by using chemical crosslinking agents; by physicochemical methods including electroporation and the like, or by other permeabilizing methodologies including, e.g., physical manipulations such as electroporation. Those skilled in the art are therefore familiar with methods for permeabilizing cells and can readily determine without undue experimentation the most appropriate permeabilizing agent for use according to the present invention, and as provided herein. Relevant factors for this determination include, but are not limited to, toxicity of the permeabilizing agent to a specific cell, the molecular size of the molecule for which entry into the cell is sought through the use of permeabilization, and the like (see, e.g., Schulz, Methods Enzymol. 192:280-300, 1990).

Thus, for instance, cells may be permeabilized using any of a variety of known techniques, including addition of permeabilizing agents such as bacterial toxins, for example, streptolysin O, Staphylococcus aureus α-toxin (α-hemolysin); other hemolytic agents such as saponin; or exposure to one or more detergents (e.g., digitonin, Triton X-100, NP-40, n-Octyl β-D-glucoside and the like) at concentrations below those used to lyse cells and solubilize membranes (i.e., below the critical micelle concentration). Certain common transfection reagents, such as DOTAP, may also be used. ATP can also be used to permeabilize intact cells, as may be low concentrations of chemicals commonly used as fixatives (e.g., formaldehyde). All of the permeabilizing agents described in this paragraph are available from, e.g., Sigma Chemical Co., St. Louis, Mo. (see Sigma catalog entitled “Biochemicals and Reagents for Life Science Research,” Anon., 1999, and references cited therein for these and other permeabilizing agents).

In another embodiment, the present invention relates to host cells containing the above described recombinant mitochondrial divalent cation transporter polypeptide expression constructs, and/or a nucleic acid expression construct which directs expression of an esterase that localizes to a mitochondrion and that is capable of cleaving a divalent cation-sensitive indicator molecule precursor to provide a divalent cation-sensitive indicator molecule. . Host cells are genetically engineered (transduced, transformed or transfected) with the vectors and/or expression constructs of this invention which may be, for example, a cloning vector, a shuttle vector or an expression construct. The vector or construct may be, for example, in the form of a plasmid, a viral particle, a phage, etc. The engineered host cells can be cultured in conventional nutrient media modified as appropriate for activating promoters, selecting transformants or amplifying particular genes such as genes encoding mitochondrial divalent cation transporter and/or esterase polypeptides or fusion proteins. The culture conditions for particular host cells selected for expression, such as temperature, pH and the like, will be readily apparent to the ordinarily skilled artisan.

The host cell can be a higher eukaryotic cell, such as a mammalian cell, or a lower eukaryotic cell, such as a yeast cell, or the host cell can be a prokaryotic cell, such as a bacterial cell. Representative examples of appropriate host cells according to the present invention include, but need not be limited to, bacterial cells, such as E. coli, Streptomyces, Salmonella tvphimurium; fungal cells, such as yeast, for example Saccahromyces cerevisiae, Schizosacchromyces pombe, Candida albicans and Pichia pastoris, insect cells, such as Drosophila S2, Trichoplusia ni (PharMingen, San Diego, Calif.) and Spodoptera Sf9; animal cells, such as CHO, COS or 293 cells; adenoviruses; plant cells, or any suitable cell already adapted to in vitro propagation or so established de novo. The selection of an appropriate host is deemed to be within the scope of those skilled in the art from the teachings herein.

In particularly preferred embodiments, the present invention is directed in pertinent part to a method wherein a nucleic acid expression construct as provided herein is contacted with a biological sample comprising a host cell that either lacks or exhibits impaired (e.g., detectably decreased in a statistically significant manner) endogenous mitochondrial calcium uniporter activity as provided herein, in order to provide advantages associated with the expression of a desired mitochondrial divalent cation transporter encoding construct. For example, detection of particular mitochondrial divalent cation transporter encoding nucleic acid sequences or transporter polypeptides that are highly similar to those encoded by the host cell genome may be facilitated by inhibiting host cell mitochondrial divalent cation transporter gene expression. As another example, where functional activity of an exogenously introduced recombinant transporter polypeptide is to be determined in a host cell or in a biological sample derived therefrom, it may also be advantageous to inhibit endogenous host cell transporter gene expression.

Thus, in certain preferred embodiments of the invention, host cells may lack at least one endogenous mitochondrial divalent cation transporter, and in certain preferred embodiments the host cells may lack all endogenous divalent cation transporter activity, whether naturally or as the result of a specifically or non-specifically induced mutation, or through genetic manipulations such as antisense nucleic acid (including targeted ribozymes) or transgenic knock-out strategies, or through pharmacological intervention. Such a host cell is preferably a yeast cell. The yeast may be Saccahromyces cerevisiae, Schizosacchromyces pombe, Candida albicans or Pichia pastoris, or any other yeast. Particularly preferred are yeast cells that lack endogenous mitochondrial calcium uniporter activity, for example, certain strains of Saccahromyces cerevisiae including those described herein.

In other preferred embodiments, the host cell may be a cell that exhibits impaired growth in the presence of a cytostatic Ca²⁺ concentration. For example, as described herein and as also disclosed by Cunningham et al. (1996 Molec. Cell. Biology 16:2226-37 and references cited therein; see also Cunningham et al., 1994 J. Cell Biol. 124:351-363 and references cited therein) S. cerevisiae single and double mutants are described that lack one or both of the vacuolar and secretory pathway (e.g., Golgi/ER) calcium-dependent ATPase activities, and which are thus unable to import Ca²⁺ into these membrane bounded organelles concomitantly with ATP hydrolysis.

In other preferred embodiments, expression in host cells of at least one gene encoding an endogenous mitochondrial divalent cation transporter polypeptide is substantially impaired. Substantial impairment of endogenous transporter polypeptide expression may be achieved by any of a variety of methods that are well known in the art for blocking specific gene expression, including site-specific or site-directed mutagenesis as described above, antisense inhibition of gene expression, ribozyme mediated inhibition of gene expression and generation of mitochondrial DNA depleted (ρ⁰) cells.

Identification of oligonucleotides and ribozymes for use as antisense agents and DNA encoding genes for targeted delivery for genetic therapy involve methods well known in the art. For example, the desirable properties, lengths and other characteristics of such oligonucleotides are well known. Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (see, e.g., Agrwal et al., Tetrehedron Lett. 28:3539-3542 (1987); Miller et al., J. Am. Chem. Soc. 93:6657-6665 (1971); Stec et al., Tetrehedron Lett. 26:2191-2194 (1985); Moody et al., Nucl. Acids Res. 12:4769-4782 (1989); Uznanski et al., Nucl. Acids Res. (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein In: Oligodeoxynucleotides. Antisense Inhibitors of Gene Expression, Cohen, Ed, Macmillan Press, London, pp. 97-117 (1989); Jager et al., Biochemistry 27:7237-7246 (1988)).

Antisense nucleotides are oligonucleotides that bind in a sequence-specific manner to nucleic acids, such as mRNA or DNA. When bound to mRNA that has complementary sequences, antisense prevents translation of the mRNA (see, e.g., U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith and Clusel et al. (1993) Nucl. Acids Res. 21:3405-3411, which describes dumbbell antisense oligonucleotides). Triplex molecules refer to single DNA strands that bind duplex DNA forming a colinear triplex molecule, thereby preventing transcription (see, e.g., U.S. Pat. No. 5,176,996 to Hogan et al., which describes methods for making synthetic oligonucleotides that bind to target sites on duplex DNA).

According to this embodiment of the invention, particularly useful antisense nucleotides and triplex molecules are molecules that are complementary to or bind the sense strand of DNA or mRNA that encodes a mitochondrial divalent cation transporter polypeptide or a protein mediating any other process related to expression of endogenous transporter genes, such that inhibition of translation of mRNA encoding the divalent cation transporter polypeptide is effected.

A ribozyme is an RNA molecule that specifically cleaves RNA substrates, such as mRNA, resulting in specific inhibition or interference with cellular gene expression. There are at least five known classes of ribozymes involved in the cleavage and/or ligation of RNA chains. Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). According to certain embodiments of the invention, any such mitochondrial divalent cation transporter mRNA-specific ribozyme, or a nucleic acid encoding such a ribozyme, may be delivered to a host cell to effect inhibition of transporter gene expression. Ribozymes, and the like may therefore be delivered to the host cells by DNA encoding the ribozyme linked to a eukaryotic promoter, such as a eukaryotic viral promoter, such that upon introduction into the nucleus, the ribozyme will be directly transcribed.

As used herein, expression of a gene encoding an endogenous mitochondrial divalent cation transporter polypeptide is substantially impaired by any of the above methods for inhibiting when cells are substantially but not necessarily completely depleted of functional DNA or functional mRNA encoding the endogenous transporter, or of the relevant transporter polypeptide. Mitochondrial divalent cation transporter expression is substantially impaired when cells are preferably at least 50% depleted of DNA or mRNA encoding the endogenous transporter (as measured using high stringency hybridization as described above) or depleted of a transporter polypeptide; and more preferably at least 75% depleted of endogenous mitochondrial divalent cation transporter DNA, mRNA or polypeptide. Most preferably, divalent cation transporter expression is substantially impaired when cells are depleted of >90% of their endogenous transporter DNA, MRNA, or polypeptide.

Alternatively, expression of a gene encoding an endogenous mitochondrial divalent cation transporter may be substantially impaired through the use of mitochondrial DNA depleted ρ⁰ cells, which are incapable of mitochondrial replication and so may not continue to express functional transporter polypeptides. Methods for producing ρ⁰ cells are known and can be found, for example in PCT/US95/04063, which is hereby incorporated by reference.

In other preferred embodiments, a host cell may be transfected with at least one exogenously derived gene encoding at least one esterase that is capable of cleaving a divalent cation-sensitive indicator molecule precursor to provide a divalent cation-sensitive indicator molecule, for example, an esterase that is capable of cleaving acetomethoxyesters. Particularly preferred are embodiments wherein such host cells are yeast cells that have been transfected to express esterases targeted to localize to mitochondria, for example, using mitochondrial targeting sequences as described herein. Without wishing to be bound by theory, according to the present invention there is provided the observation that many yeast cells lack one or more, or all, esterase activities commonly found in many mammalian cells, which esterase activities convert many of the ester-containing precursor forms of divalent cation-sensitive indicator molecules as provided herein (e.g., fluorescent indicators such as Rhod-2, Fura-2, Indo-1, Fluo-3, which are available from Molecular Probes, Inc., see Haugland, 1996 Handbook of Fluorescent Probes and Research Chemicals-Sixth Ed., Molecular Probes, Eugene, Oreg.) into useful fluorophores. Thus, for example, there are known to the art a number of bacterially derived esterases that are capable of cleaving ester linkages such as the acetomethoxyester linkage present in many such indicator molecules. Accordingly, the present invention contemplates the use of a transfected cell that expresses at least one such esterase, as a host cell, in the method for identifying a nucleic acid molecule encoding a mitochondrial divalent cation transporter polypeptide, as provided herein.

Selection of a suitable esterase for use in these and related embodiments of the present invention may be achieved according to the disclosure provided herein, and using compositions and methods known in the art. A suitable esterase preferably (i) cleaves a divalent cation-sensitive indicator molecule precursor (e.g., containing a hydrolyzable ester linkage such as an acetomethoxyester linkage) to provide a divalent cation-sensitive indicator molecule, (ii) localizes to mitochondria and (iii) is desirably non-toxic to host cells to be used in the subject invention method.

Typically, cleavage of a divalent cation-sensitive indicator molecule precursor, such as a precursor containing an ester linkage, may be monitored as a shift in spectral properties (e.g., fluorescence excitation/emission spectra) of the indicator molecule that accompanies its conversion from precursor to indicator molecule, according to well known methodologies that will vary depending on the particular indicator molecule employed (see, e.g., Haugland et al., 1996, supra). For example, the B. subtilis esterase described in greater detail in the Examples below exhibited the ability to cleave ester-containing precursors of the cation-sensitive indicator molecules Rhod-2, Fluo-3 and Indo-1.

Mitochondrial localization of an esterase may be achieved through the use of a mitochondrial targeting sequence as provided herein; confirmation of mitochondrial localization of the enzyme may be obtained by any of a number of procedures known to the art, including subcellular localization through detection in situ (e.g., through imaging such as fluorescence, immunohistochemical, confocal or electron microscopy) or via cell fractionation into discrete subcellular fractions including a mitochondria-enriched fraction. Thus, for example, cell fractionation techniques for the enrichment and detection of mitochondria, and/or biochemical markers characteristic of these and other defined organelles, may be used to determine that a particular subcellular fraction containing one or more detectable organelle-specific or organelle-associated markers or polypeptides is substantially enriched in mitochondria (see, e.g., Emster et al., 1981 J. Cell Biol. 91:227s). Enzymatic activities of organelle-specific marker enzymes in fractions so derived may be determined using well known methodologies, for example, according to the methods of Storrie et al. (1990 Meths. Enzymol. 182:203), wherein marker enzyme activities for the mitochondrial, lysosomal, Golgi and plasma membrane compartments may be determined.

Those having ordinary skill in the art will further readily appreciate that any number of procedures exist for determining whether a chemical compound is toxic to a host cell according to methods for detecting cell viability (e.g., vital dye exclusion, lactate dehydrogenase release, etc.). Accordingly it will be appreciated that, as known in the art and as disclosed herein, a suitable esterase for use in the present invention may be selected readily and without undue experimentation. As a non-limiting example, a para-nitrobenzyl esterase from Bacillus subtilis, or a nucleic acid molecule encoding same, may be selected for use according to the present invention. Such esterases and esterase encoding nucleic acids, including selected esterase variants exhibiting greater thermostability, are disclosed in U.S. Pat. Nos. 5,741,961; 5,906,930; 5,945,325; Zock et al., 1994 Gene 151:37-43; and Spiller et al., 1999 Proc. Nat. Acad. Sci. USA 96:12305-10.

Various mammalian cell culture systems can also be employed to express recombinant protein. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences, for example as described herein regarding the preparation of mitochondrial divalent cation transporter polypeptide expression constructs. DNA sequences derived from the SV40 splice, and polyadenylation sites may be used to provide the required nontranscribed genetic elements. Introduction of the construct into the host cell can be effected by a variety of methods with which those skilled in the art will be familiar, including but not limited to, for example, calcium phosphate transfection, DEAE-Dextran mediated transfection, or electroporation (Davis et al., 1986 Basic Methods in Molecular Biology).

Functional Screening Assays

In certain embodiments the invention provides methods for identifying the presence of a mitochondrial divalent cation transporter (e.g., calcium uniporter) expressed in a host cell by a nucleic acid expression constructs, by functionally detecting cation transport across a mitochondrial inner membrane within the host cell. As a non-limiting example, calcium uniporter activity expressed from a nucleic acid expression construct may be detected as a gain of function in an organelle, cell or organism known to lack such function (e.g., S. cerevisiae). Such methods may also be useful for diagnostic and prognostic purposes, such as in the determination of the existence of altered mitochondrial function which, as described above, may accompany both normal and disease states.

In certain aspects the invention provides a method for identifying a nucleic acid encoding a mitochondrial divalent cation transporter polypeptide, which comprises (a) contacting a biological sample comprising a host cell containing at least one mitochondrion with at least one nucleic acid expression construct under conditions and for a time sufficient to permit expression of at least one mitochondrial divalent cation transporter polypeptide, wherein (i) the mitochondrion comprises a divalent cation-sensitive indicator molecule that is capable of generating a detectable signal in the presence of a divalent cation, and (ii) the nucleic acid expression construct comprises a promoter operably linked to a nucleic acid encoding a candidate mitochondrial divalent cation transporter polypeptide; (b) exposing the host cell to a divalent cation under conditions and for a time sufficient to permit transport of the divalent cation across a membrane by the (expressed) candidate mitochondrial divalent cation transporter polypeptide; and (c) detecting a signal generated by the divalent cation-sensitive indicator molecule in at least one mitochondrion.

According to certain embodiments, an interaction between the divalent cation and the expressed transporter polypeptide includes any contact, binding, or association that leads to the transport of divalent cation from one cell compartment (e.g., cytoplasm) to another (e.g., mitochondrion), as detected by a the signal generated by the divalent cation-sensitive indicator molecule. Generation of such a signal may arise, according to non-limiting theory, from any contact, binding, association or other interaction between the divalent cation and the divalent cation-sensitive indicator molecule that leads to a detectable change in fluoresence, phosphoresence, bioluminesence, or the like (depending on the indicator selected), relative to the absence of such interaction, as an indication of divalent cation transport from one cell compartment (e.g., cytoplasm) to another (e.g., mitochondrion).

A “divalent cation-sensitive indicator molecule” may include any enzyme, metabolic protein, marker protein (including fluorescent proteins) or other naturally occurring or synthetic detectable marker that is capable of generating a detectable signal as an indication of the presence of a divalent cation, preferably in proportion to the amount, level or conentration of the divalent cation that is present. Thus, the invention pertains in part to detecting a signal generated by a calcium indicator molecule in a mitochondrion as provided herein. The calcium indicator molecule may be endogenous to (e.g., naturally occurring in) the mitochondrion or it may be exogenous, which includes at least one calcium indicator molecule that does not occur naturally in the mitochondrion but that has been loaded, administered, admixed, expressed (including expression as the product of a genetically engineered nucleic acid construct), targeted, contacted, exposed or otherwise artificially introduced into the mitochondrion, as long as the calcium indicator molecule is capable of generating a detectable signal that is proportional to the level of calcium. In certain embodiments the calcium indicator molecule is exogenous and the detectable signal is a fluorescent signal.

As provided herein, a divalent cation-sensitive indicator molecule precursor includes any compound that can be converted into a divalent cation-sensitive indicator molecule; it is preferred that this conversion take place within a host cell and more preferably within a mitochondrion within such host cell. A divalent cation-sensitive indicator molecule precursor is preferably cleaved by an esterase, as described herein (and preferably a mitochondrially localized esterase), under conditions and for a time sufficient for esterase enzyme catalytic activity to be manifested, to provide a divalent cation-sensitive indicator molecule. The divalent cation-sensitive indicator molecule precursor of the present invention is therefore preferably a precursor that includes an ester linkage, and still more preferably a precursor that includes an acetomethoxyester linkage (see, e.g., Haugland et al., 1996, supra), although the invention need not be so limited. In particularly preferred embodiments the divalent cation-sensitive indicator molecule precursor is capable of crossing a cellular membrane as provided herein, where such capability may reflect passive diffusion, facilitated diffusion, active transport, pinocytotic, phagocytic absorptive or adsorptive endocytosis or other endocytic processes, or any other mechanism or process whereby the precursor molecule may undergo transit from one side of a cellular membrane to the other. Thus, by way of illustration and not limitation, in certain preferred embodiments of the present invention, a divalent cation-sensitive indicator molecule precursor may be introduced extracellularly prior to traversal of the plasma membrane to the intracellular environment, where diffusion to mitochondrial sites permits catalytic cleavage of the precursor by a mitochondrially localized esterase to provide a mitochondrial divalent cation-sensitive indicator molecule.

Where the divalent cation-sensitive (e.g., calcium) indicator molecule is a fluorescent indicator, the signal generated by the indicator molecule, which signal is proportional to the level of calcium in the mitochondrion, may be detected by exposing the sample to light having an appropriate wavelength to excite the indicator, and determining resultant fluorescence with a suitable instrument for detecting a fluorescent light emission at an appropriate wavelength. Those having ordinary skill in the art can readily determine the manner by which the sample is contacted with the source of calcium cations based on the teachings provided herein, in view of the properties of the sample (including the calcium indicator molecules selected) and those of the source of calcium ions selected. As discussed in greater detail below, the method of the present invention may be used to identify a mitochondrial calcium uniporter.

Thus, in preferred embodiments the divalent cation-sensitive (e.g., calcium) indicator molecule may be a light emission molecule, for example a fluorescent, phosphorescent, or chemiluminescent molecule or the like, which emits a detectable signal in the form of light when excited by excitation light of an appropriate wavelength. “Fluorescence” refers to luminescence (emission of light) that is caused by the absorption of radiation at one wavelength (“excitation”), followed by nearly immediate re-radiation (“emission”), usually at a different wavelength, that ceases almost at once when the incident radiation stops. At a molecular level, fluorescence occurs as certain compounds, known as fluorophores, are taken from a ground state to a higher state of excitation by light energy; as the molecules return to their ground state, they emit light, typically at a different wavelength. “Phosphorescence,” in contrast, refers to luminescence that is caused by the absorption of radiation at one wavelength followed by a delayed re-radiation that occurs at a different wavelength and continues for a noticeable time after the incident radiation stops. “Chemiluminescence” refers to luminescence resulting from a chemical reaction, and “bioluminescence” refers to the emission of light from living organisms or cells, organelles or extracts derived therefrom.

A variety of calcium indicators are known in the art and may be suitable for generating a detectable intracellular signal, for example, a signal that is proportional to the level of calcium, including but not limited to fluorescent indicators such as Fura-2 (McCormack et al., 1989 Biochim. Biophys. Acta 973:420); mag-fura-2; BTC (U.S. Pat. No. 5,501,980); fluo-3, fluo-4, fluo-5F and fluo-5N (U.S. Pat. No. 5,049,673); fura-4F, fura-5F, fura-6F, and fura-FF; Rhod-2, Rhod-5F; Indo-1; Calcium Green 5N; benzothiaza-1 and benzothiaza-2; and others, which are available from Molecular Probes, Inc., Eugene, OR (see also, e.g., Calcium Signaling Protocols—Meths. In Mol. Biol.—Vol. 114), Lambert, D. (ed.), Humana Press, 1999). Precursor forms containing cleavable ester linkages are available for many of these indicator molecules.

Calcium Green SN is a particularly preferred calcium indicator molecule for use according to the present invention. Depending, however, on the particular assay conditions to be used, a person having ordinary skill in the art can select a suitable calcium indicator from those described above or from other calcium indicators, according to the teachings herein and based on known properties (e.g., solubility, stability, etc.) of such indicators. For example by way of illustration and not limitation, whether a cell permeant or cell impermeant indicator is needed (e.g., whether a sample comprises a permeabilized cell), affinity of the indicator for calcium (e.g., dynamic working range of calcium concentrations within a sample as provided herein), subcellular localization of the indicator (which may in turn depend on other properties of the host cell, such as one or more expressed, transfected components) and/or fluorescence spectral properties such as a calcium-dependent fluorescence excitation shift, may all be factors in the selection of a suitable calcium indicator.

A variety of instruments can be used in methods of the invention to excite a calcium indicator molecule as provided herein that is a fluorescent compound, and to detect the signal generated by the calcium indicator molecule that is proportional to the level of calcium, e.g., to measure the resulting emission therefrom. Selection of a suitable instrument, light source, filter set, etc. may depend on factors known to those familiar with the art, such as (i) application of energy (i.e., light) at a wavelength that will excite the calcium indicator molecule, preferably at or near the optimum excitation wavelength of the indicator molecule (λmax₍ₑₓ₎); (ii) detection of energy (ie., light) within the emission spectrum of the acceptor compound, preferably at or near the optimum emission wavelength of the indicator molecule (λmax₍ₑₘ₎); (iii) the type of samples to be assayed; and (iv) the number and formatting of samples to be assayed in a given program, for example, a high throughput screening format.

With regard to factors (i) and (ii), the spectra of energy being applied to, and the spectra of energy being emitted by the samples will determine, in general, what type of instrument will be used. For example, although λ(ex) should not be identical to λ(em), the minimal acceptable amount of difference between these two values will be influenced by, among other factors, the instrumentation being used. That is, as λ(ex) approaches λ(em), instruments capable of resolving closely-spaced wavelengths are required, and an assay wherein the difference between λ(ex) and λ(em) is less than about 3 to about 5 nm requires a high resolution instrument. Conversely, an assay wherein the difference between λ(ex) and λ(em) is greater than about 50 to about 75 nm requires an instrument having relatively medium to low resolution.

Thus, with specific regard to factor (ii), the type of energy being emitted by an excited fluorophore and measured in samples will determine, in general, what type of instrument will be used. A fluorometer, for instance, is a device that measures fluorescent energy and should therefore be part of the instrumentation. A fluorometer may be anything from a relatively simple, manually operated instrument that accommodates only a few reaction vessels (e.g., sample tubes) at a time, to a somewhat more complex manually operated or robotic instrument that accommodates a larger number of samples in a format having a plurality of reaction vessels, such as a 96-well microplate (e.g., an fmax™ fluorimetric plate reader, Molecular Devices Corp., Sunnyvale, Calif.; or a Cytofluor™ fluorimetric plate reader, model #2350, Millipore Corp., Bedford, Mass.), or a complex robotic instrument (e.g, a FLIPR™ instrument; see infra) that accommodates a multitude of samples in a variety of formats such as 96-well microplates, 384-well microplates or other high throughput screening formats wherein, for example, detection of signals from a calcium indicator molecule in a plurality or reaction vessels may be automated.

With regard to factor (iii), the type of samples to be assayed in a given program, different formats will be appropriate for different types of samples. For example, 96-well or 384-well microplates may be suitable in instances where the cells of interest adhere to the microplate substrate, or to some material applied to the wells of the microplate (e.g., a natural or synthetic coating with which the wells have been treated, such as collagen, fibronectin, vitronectin, RGD peptide, poly-L-lysine, CelTak™, or the like). Interfering fluorescence derived from certain common plastic multiwell plate materials, however, may result in a large artifactual background component at excitation wavelengths below about 400 nm. Accordingly, for measurements involving nonadherent cells such as suspension cells, or suspensions of adherent cells that have been dislodged from a growth substrate, or suspension of adherent cells on microcarriers or the like, an instrument capable of reading fluorescent signals in glass or polymeric tubes or tubing, or another suitable non-interfering vessel, may be preferred. Regardless of what type of format is used, assay reaction vessels should allow for the introduction of biological samples, candidate agents, a source of calcium cations, control reagents and optionally additional compounds that may influence cytosolic calcium levels, as well as the ability to detect the signal generated by the calcium indicator molecule at a plurality of appropriate points in time.

Factor (iv), the number of samples to be assayed in a given program, may influence the degree of automation that can be implemented by the instrument selected. For example, when high throughput (HTS) screening, (i.e., assaying a large number of samples in a relatively brief time period) is desired, robotic or semi-robotic instruments are preferred. Alternatively, samples may be processed manually, even where formats that accommodate large sample numbers (e.g., 96-well microplates) are used.

Preferred divalent cation-sensitive indicator molecules include aequorin proteins (see, e.g., U.S. Pat. Nos. 5,360,728; 5,162,227; 5,422,266; 5,093,240; 5,139,937), luciferase, green fluorescent protein and its derivatives (GFP; see, e.g., U.S. Pat. Nos. 5,491,084; 5,777,079; 5,625,048; 5,804,387; 5,776,681; 5,741,668), and an appropriately selected, divalent cation-sensitive cognate recognition molecule for use with a FLASH sequence that may be engineered to be present in a polypeptide localized to a mitochondrion (for details on FLASH sequences, see, e.g., U.S. Pat. No. 5,932,474; Griffin et al., 1998 Science 281:269). In certain embodiments, the divalent cation-sensitive indicator molecule is aequorin, which in some preferred embodiments has been expressed in such a way as to localize to the mitochondrion, for example, by virtue of its expression as a fusion protein bearing a mitochondrial targeting sequence such as the human cytochrome c oxidase subunit VIII (Rizzuto et al., 1995 Meths. Enzymol. 260:417) or another mitochondrial targeting sequence. For instance, yeast mitochondria are known to express, import, and process other mitochondrial proteins from mammalian sources (e.g., Scarpulla et al., 1986 Proc. Nat. Acad. Sci. USA 83:6352; Murdza-Inglis et al., 1991 J. Biol. Chem. 266:11871; Koshy et al., 1992 Prot. Express. Purific. 3:441). In further preferred embodiments, the aequorin may be recombinantly expressed from a plasmid, such as mtAEQ/pMT2 or mtAEQ/pYES2. Accordingly, the instant method for monitoring and detecting divalent cation transport between subcellular compartments or into and out of cells, will be useful for monitoring the presence (i.e., expression) of candidate mitochondrial divalent cation transporter polypeptide(s). In other preferred embodiments, the host cell may be further contacted with an aequorin co-factor to aid detection as provided herein, such as native coelenterazine, coelenterazine f, coelenterazine h, coelenterazine hcp, or coelenterazine n.

As provided herein, detection employs a divalent cation-sensitive indicator molecule capable of generating a detectable signal that corresponds to the local divalent cation concentration. As noted above and provided herein, aequorin is a calcium regulated photoprotein that will emit light in a cell at 488 nm due to co-factor consumption resulting from an interaction involving aequorin and calcium. By way of illustration and not limitation, a host cell, harboring intramitochondrial aequorin and expressing a nucleic acid expression construct encoding a mitochondrial calcium uniporter, may be contacted with calcium and coelenterazine to detect such a calcium-aequorin interaction as a measure of uniporter activity, by virtue of conditions as described herein permitting mitochondrial uptake of calcium via a calcium uniporter. In certain preferred embodiments, a variety of standard detection techniques may be used to determine the presence of an interaction between a mitochondrion divalent cation-sensitive indicator molecule and a divalent cation, including fluorimetry, fluorescence resonance energy transfer (FRET, see Haugland, 1996); flow cytoflourimetry, including fluorescence activated cell sorting (FACS); chemiluminesence, radiometry (e.g., detection of [⁴⁵Ca] radionuclide beta decay) and spectrophotometry. The term “screen for” or “screening” refers to the use of the subject invention compositions and methods to identify nucleic acid expression constructs encoding mitochondrial divalent cation transporter, binding, and/or regulatory polypeptide(s).

A candidate nucleic acid expression construct identified as provided herein may be recombinantly expressed in a host cell and functionally characterized by determining calcium binding, calcium transport, or other calcium regulatory activity in an organelle, cell, or other material prepared therefrom. In certain preferred embodiments, the nucleic acid expression construct may be extrachromosomal or integrated into a host cell chromosome, such as a nuclear or mitochondrial chromosome. As described above, preferably the host cell is contacted with a genetic library of nucleic acid expression constructs, where the population of constructs includes a vector with nucleic acid inserts, such as genomic DNA, cDNA synthesized from total RNA or cDNA synthesized from mRNA. Host cells will take up and express the nucleic acid constructs through techniques known in the art and described herein, such as transfection or transformation, and constructs having polynucleotide sequences encoding transporter activity may be detected, isolated, and identified as described herein. Preferably, the mitochondrial divalent cation transporter polypeptide(s) comprises a uniporter, and most preferably a calcium uniporter.

As noted above, a host cell may include any eukaryotic or prokaryotic cell and will, in certain preferred embodiments, lack any detectable calcium transporter (e.g., uniporter) activity, such as the yeast cells of the Sacchromyces cerevisiae strain described herein, or their equivalents. As also described above, a host cell preferably includes at least one mitochondrion, and the mitochondrion comprises at least one divalent cation-sensitive indicator molecule as provided herein, for example, aequorin. In certain other preferred embodiments, the divalent cation may be any cation known or described herein, such as barium, calcium, cobalt, iron, lanthanide series member (including lanthanum), lead, magnesium, manganese, strontium, or zinc. Most preferably the cation comprises calcium.

Preferably the calcium is at a concentration from about 0.01 μM to about 100 μM, more preferably from about 0.1 μM to about 50 μM, and most preferably from about 0.5 μM to about 15 μM, where “about” as used herein means ±10%.

In another aspect the invention provides a method for identifying a nucleic acid encoding a mitochondrial divalent cation transporter polypeptide, comprising (a) contacting a host cell with at least one nucleic acid expression construct under conditions and for a time sufficient to permit expression of at least one mitochondrial divalent cation transporter polypeptide, wherein (i) host cell growth is impaired in the presence of Ca2+, and (ii) the nucleic acid expression construct comprises a promoter operably linked to a nucleic acid encoding a candidate mitochondrial divalent cation transporter polypeptide; (b) exposing the host cell to a divalent cation under conditions and for a time sufficient to permit transport of the divalent cation across a membrane by the candidate mitochondrial divalent cation transporter; and (c) detecting cell growth in at least one host cell, and therefrom identifying a nucleic acid encoding a mitochondrial divalent cation transporter polypeptide.

Accordingly, in related embodiments the present invention contemplates the use of a host cell, the growth of which is impaired in the presence of Ca²⁺. For example, such a host cell may have one or more mutated calcium/ATPase genes, such that Ca²⁺ is cytostatic, substantially impairs growth and/or may be toxic to the cells, depending on the calcium concentration. Such ATPase genes may include any known ATPase gene that encodes a functional ATPase pump polypeptide as known in the art and provided herein. Preferred ATPase genes are those that encode divalent cation ATPase pumps, which may be localized, for example, to cellular membranes of specific subcellular compartments, such as the yeast vacuolar calcium ATPase gene PMCI and the secretory organelle (e.g., endoplasmic reticulum) calcium ATPase gene PMR1 (Cunningham et al., 1996, Molec. Cell. Biology 16:2226-37; Cunningham et al., 1994 J. Cell Biol. 124:351-363).

Single (e.g., PMC1) and double (e.g., PMC1/PMR1) ATPase mutants, or other host cells exhibiting impaired growth in the presence of Ca²⁺, may vary with respect to their calcium sensitivity, but those having ordinary skill in the art can readily and without undue experimentation determine what are cytostatic calcium concentrations for such cells. Such determination may be achieved using any of a wide variety of methods for determining whether cell growth is present, including visual examination of macroscopic colonies, microscopy, quantification of cell number using, e.g., a hemacytometer or flow cytometry, viability determination by vital dye exclusion or enzyme activity assays, or any other suitable method. For example, Ca²⁺ concentrations ≦300 μM are cytostatic for PMC1/PMR1 double ATPase mutant cells (Cunningham et al., 1994), while the presence of calcium that impairs the growth of other host cells may vary with the cell selected. Impaired growth in the presence of Ca²⁺ thus may refer to cytostasis, i.e., a cessation of cell division, in certain preferred embodiments, and in certain other embodiments may refer to a cell growth rate that is less than 70% of the rate for wild type counterpart cells, more preferably less than 40%, still more preferably less than 25%, and still more preferably less than 15%. In certain other embodiments, the host cell contains a vacuolar assembly mutant whereby vacuolar Ca²⁺ transport is disabled (Miseta et al., 1999 J. Biol. Chem. 274:5939).

Upon identification, isolation, and sequencing of a nucleic acid expression construct encoding a candidate mitochondrial divalent cation transporter (e.g., calcium uniporter) polypeptide(s), further determination of a calcium uniporter role in cellular calcium homeostasis may be made by detecting a specific loss of uniporter function, for example, in cells that lack a uniporter polypeptide and/or cells in which uniporter expression is substantially impaired as provided herein, for instance, as a consequence of exposure to a uniporter-specific antisense reagent or ribozyme, as described above. In certain other aspects, the present invention provides a method for preparing mitochondrial divalent cation transporter polypeptide(s) identified as described herein. Preferably, a host cell containing a candidate nucleic acid expression construct that encodes a mitochondrial divalent cation transporter polypeptide(s) (e.g., calcium uniporter) is cultured under conditions and for a time sufficient to permit expression of the polypeptide(s). To facilitate isolation, the nucleic acid constructs may encode additional polypeptide sequences added to a transporter to facilitate detection and isolation of transporter polypeptides, such as fusion protein affinity tag sequences described above.

Other functional properties of a calcium uniporter may also be tested in suitable cell-based and cell-free systems for assaying mitochondrial activities as provided herein and with which those skilled in the art will be familiar, including but not limited to: activation by ADP, inhibition by ATP, Mg²⁺, ruthenium red or its derivative Ru360 (Matlib et al., 1998 J. Biol. Chem. 273:10223; Emerson et al., 1993 J. Amer. Chem. Soc. 115:11799) and competitive inhibition by, for example, Sr²⁺, Mn²⁺, Ba²⁺, Co2+, Fe²⁺, Pb²⁺, Zn2+ and lanthanide series members, e.g., La³⁺.

As also provided herein, calcium uniporter activity may be determined as it relates to mitochondrial membrane potential, which under certain conditions may be altered (e.g., increased or decreased in a statistically significant manner) in response to calcium transport by a functional uniporter. Methods for determining mitochondrial membrane potential are provided, for example, in U.S. application Ser. No. 09/161,172. Typically, mitochondrial membrane potential may be determined according to methods with which those skilled in the art will be readily familiar, including but not limited to detection and/or measurement of detectable compounds such as fluorescent indicators, optical probes and/or sensitive pH and ion-selective electrodes (See, e.g., Emster et al., 1981 J. Cell Biol. 91:227s and references cited; see also Haugland, 1996 Handbook of Fluorescent Probes and Research Chemicals—Sixth Ed., Molecular Probes, Eugene, Oreg., pp. 266-274 and 589-594.). For example, by way of illustration and not limitation, the fluorescent probes 2-,4-dimethylaminostyryl-N-methyl pyridinium (DASPMI) and tetramethylrhodamine esters (such as, e.g., tetramethylrhodamine methyl ester, TMRM; tetramethylrhodamine ethyl ester, TMRE) or related compounds (see, e.g., Haugland, 1996, supra) may be quantified following accumulation in mitochondria, a process that is dependent on, and proportional to, mitochondrial membrane potential (see, e.g., Murphy et al., 1998 in Mitochondria & Free Radicals in Neurodegenerative Diseases, Beal, Howell and Bodis-Wollner, Eds., Wiley-Liss, New York, pp. 159-186 and references cited therein; and Molecular Probes On-line Handbook of Fluorescent Probes and Research Chemicals, at http://www.probes.com/handbook/toc.html). Other fluorescent detectable compounds that may be used in the invention include but are not limited to rhodamine 123, rhodamine B hexyl ester, DiOC₆(3), JC-1[5,5′,6,6′-Tetrachloro-1,1′,3,3′-Tetraethylbezimidazolcarbocyanine Iodide] (see Cossarizza, et al., 1993 Biochem. Biophys. Res. Comm. 197:40; Reers et al., 1995 Meth. Enzymol. 260:406), rhod-2 (see U.S. Pat. No. 5,049,673; all of the preceding compounds are available from Molecular Probes, Eugene, Oregon) and rhodamine 800 (Lambda Physik, GmbH, Göttingen, Germany; see Sakanoue et al., 1997 J. Biochem. 121:29).

Mitochondrial membrane potential can also be measured by non-fluorescent means, for example by using TTP (tetraphenylphosphonium ion) and a TTP-sensitive electrode (Kamo et al., 1979 J. Membrane Biol. 49:105; Porter and Brand, 1995 Am. J Physiol. 269:R1213). Those skilled in the art will be able to select appropriate detectable compounds or other appropriate means for measuring ΔΨm. By way of example and not limitation, TMRM is somewhat preferable to TMRE because, following efflux from mitochondria, TMRE yields slightly more residual signal in the endoplasmic reticulicum and cytoplasm than TMRM.

As another non-limiting example, membrane potential may be additionally or alternatively calculated from indirect measurements of mitochondrial permeability to detectable charged solutes, using matrix volume and/or pyridine nucleotide redox determination combined with spectrophotometric or fluorimetric quantification. Measurement of membrane potential dependent substrate exchange-diffusion across the inner mitochondrial membrane may also provide an indirect measurement of membrane potential. (See, e.g., Quinn, 1976, The Molecular Biology of Cell Membranes, University Park Press, Baltimore, Md., pp. 200-217 and references cited therein.)

The invention is not intended to be limited to these examples of functionally testing a nucleic acid expression construct expressing calcium uniporter polypeptide activity, and may include other methodologies for determining calcium binding, calcium transport or other calcium regulatory activities.

Protein Production

The expressed recombinant mitochondrial divalent cation transporter polypeptides or fusion proteins may be useful in intact host cells; in intact organelles such as mitochondria, chloroplasts, vacuoles, cell membranes, intracellular vesicles including secretory pathway components such as Golgi and/or endoplasmic reticulum elements, other cellular organelles; or in disrupted cell preparations including but not limited to cell homogenates or lysates, submitochondrial particles, uni- and multilamellar membrane vesicles or other preparations. Alternatively, expressed recombinant mitochondrial divalent cation transporter polypeptides or fusion proteins can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

The divalent cation transporter polypeptides of the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated. Polypeptides of the invention may also include an initial methionine amino acid residue.

### EXAMPLES

The following Examples are offered by way of illustration and not by way of limitation.

### Example 1

### Characterization of ETH 129, an Electrogenic Ionophore, using Synthetic Phospholipid Vesicles

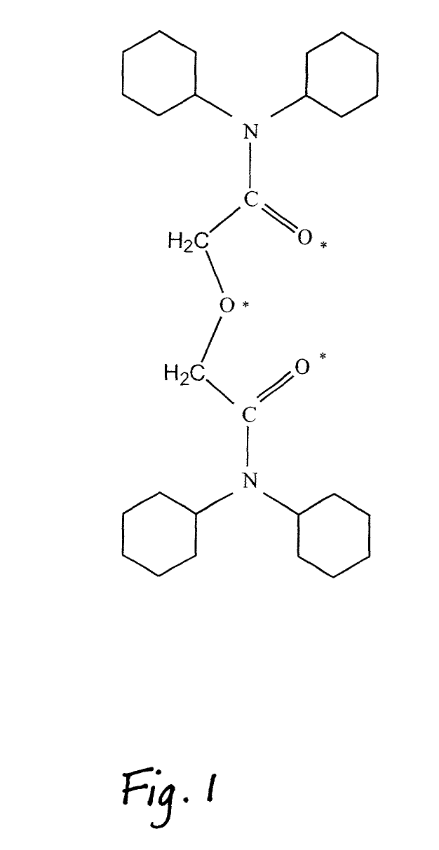

This example describes the transport mechanism and specificities of ETH 129, a calcium ionophore (Pretsch et al., 1980 Helvetica Chimica Acta 63:191-196; Prestipino et al., 1993 Anal. Biochem. 210:119) that contains no ionizable functionalities and thus forms complexes with Ca²⁺ that carry a net positive charge. The structure of ETH 129 is shown in FIG. 1, wherein asterisks denote cation liganding atoms. Ionophores of this type are referred to as electrogenic, meaning that transmembrane charge movement occurs as the ionophore transports cations across a membrane (see, e.g., Dobler, 1981, Classification of Ionophores, In Ionophores and Their Structures, John Wiley and Sons, New York, N.Y.; Westley, 1982, Notation and Classification, In Polyether Antibiotics: Naturally Occurring Acid Ionophores (Westley, J. W., editor), Marcel Dekker, New York, N.Y.). Such electrogenic ionophores may thus be distinguished from electroneutral calcium ionophores such as A23187 or ionomycin, which do not carry a net charge and which transport cations (e.g., Ca²⁺) by a mechanism in which Ca²⁺ is exchanged for another cationic species (e.g., H⁺) such that no net charge movement across a membrane accompanies the transport of Ca²⁺ across the membrane (Dobler, 1981; Westley, 1982). ETH 129 permits yeast mitochondria to accumulate large amounts of Ca²⁺ (Jung et al., 1997 J. Biol Chem. 272:21104) even though these mitochondria, unlike mammalian mitochondria, lack an endogenous activity for the inward transport of the Ca²⁺ cation; i.e., in certain yeast species, mitochondria lack a calcium uniporter. Electrogenic ionophores can also be distinguished from electroneutral ionophores in that membrane potential is a significant factor in transmembrane cation transport mediated by an electrogenic ionophore, whereas transmembrane pH gradients play a more significant role in transrrembrane cation transport mediated by electroneutral ionophores (Wooley et al., 1995, The Use of lonophores for Manipulating Intracellular Ion Concentrations, In Methods in Neurosciences (Krajcer, J. and Dixon, S. J., eds.), Academic Press, Orlando, Fla.).

Preparation of Phospholipid Vesicles.

Unilamellar vesicles loaded with Quin-2 were prepared from 1-palmitoyl-2-oleoyl-sn-glycerophosphatidylcholine (POPC) by freeze-thaw extrusion (Erdahl et al., 1994 Biophys. J. 66:1678; Erdahl et al., 1995 Biophys. J. 69:2350). The formation medium contained 5 mM Quin-2 (K+), 10 mM Hepes (K+), pH 7.00, and 100 mM KCl. A high internal K+ concentration was sought to allow the generation of membrane potentials of varying magnitude (see below). The preparations obtained were applied to Sephadex™ G-50 mini-columns (Pharmacia, Piscataway N.J.) and eluted by low speed centrifugation (Fry et al., 1978 Anal. Biochem. 90:809), to replace the external medium with a 10 mM Hepes buffer (Na+), pH 7.00 (Erdahl et al., 1994 Biophys. J. 66:1678; Erdahl et al., 1995 Biophys. J. 69:2350). The nominal concentration of POPC in final preparations was determined as lipid phosphorus (Bartlett, 1959 J Biol. Chem. 234:466) and was near 80 mM. The average diameter of these vesicles was 71 nm as determined by freeze-fracture electron microscopy (Chapman et al., 1990 Chem. Phys. Lipids 55:73). They contained entrapped K+ at approximately 125 mM, as determined by atomic absorption spectroscopy, and Quin-2 at approximately 6 mM. The latter value was determined by titrating lysed vesicles with a standard solution of CaCl₂ and was about one third of the expected value (Erdahl et al., 1995 Bipohys. J. 69:3250). The high solute level in the formation medium reduced the efficiency of Quin-2 entrapment by reducing the effectiveness of a freeze-thaw driven solute concentrating mechanism that normally elevates the internal concentrations of solutes, relative to the external medium (Chapman et al., 1990 Chem. Phys. Lipids 55:73; Chapman et al., 1990 Chem. Phys. Lipids 60:201).

Determination of Cation Transport.

POPC vesicles containing Quin-2 were utilized at a nominal phospholipid concentration of 1.0 mM, and at 25° C. The external medium contained polyvalent cation salts as described below, 100 mM NaCl, or mixtures of NaCl and KCl totaling 200 mM, and 10 mM Hepes, pH 7.00, unless otherwise noted. The medium pH was adjusted with NaOH that had been passed over Chelex 100 columns to remove contaminating cations (Erdahl et al., 1994 Biophys. J. 66:1678). In most cases an inside negative membrane electrical potential was present during the experiments. This was generated by the presence of 0.5 μM valinomycin (Val), which transports K+ out of the vesicles electrogenically (Erdahl et al., 1994 Biophys. J. 66:1678). The magnitude of the potential was controlled by varying the concentration ratio of Na+ to K+ in the external medium. A TPP+electrode was used to measure the potential (Broekemeier et al, 1998 Biochem. 37:13059), and thereby to verify that the calculated values were obtained. For these purposes the incubation medium contained 2.0 μM TPP.Cl and the entrapped volume was taken to be 2.02 μl/ml at a nominal POPC concentration of 1.0 mM (Chapman et al., 1990 Chem. Phys. Lipids 55:73).

Electrode calibration experiments conducted in the presence and absence of vesicles showed that binding of the probe to POPC was not significant. In the several preparations examined, the observed potential showed a near Nernstian relationship to variations in the transmembrane K+ gradient (observed values changed by 56-60 mV per 10-fold change in the gradient). Val was omitted when an imposed membrane potential was not desired, or in some cases 5 μM of the protonophore carbonyl cyanide m-chlorophenylhydrazone (CCP) was present instead of Val. The former condition allowed formation of a membrane potential due to the action of ETH 129, whereas the latter condition did not.

The transport of polyvalent cations was monitored by difference absorbance measurements which detected formation of the Quin-2:cation complex within the vesicle lumen. An Aminco DW2a spectrophotometer operated in the dual wavelength mode was employed. In addition, an Oriel No. 59800 band pass filter was used between the cuvette and the beam scrambler-photomultiplier assembly to prevent detection of the fluorescent light emitted by Quin-2. The sample wavelength for all cations was 264 nm. The reference wavelengths were at an isosbestic point in the Quin-2/Quin-2:cation complex difference spectrum of interest. These wavelengths varied slightly from cation to cation, as previously described (Erdahl et al., 1996 Biochem. 35:13817). Data were collected on disk using Unkel Scope software (Unkel Software, Inc., Lexington, Mass.) or the software system from On Line Instruments Systems Inc. (Bogart, Ga.) which accompanies that supplier's modification package for Aminco DW2 series spectrophotometers.

Other Methods.

Procedures used to obtain initial rates from the progress curves were as described by Erdahl et al. (1994 Biophys. J. 66:1678). Stock solutions of all ionophores were prepared in ethanol.

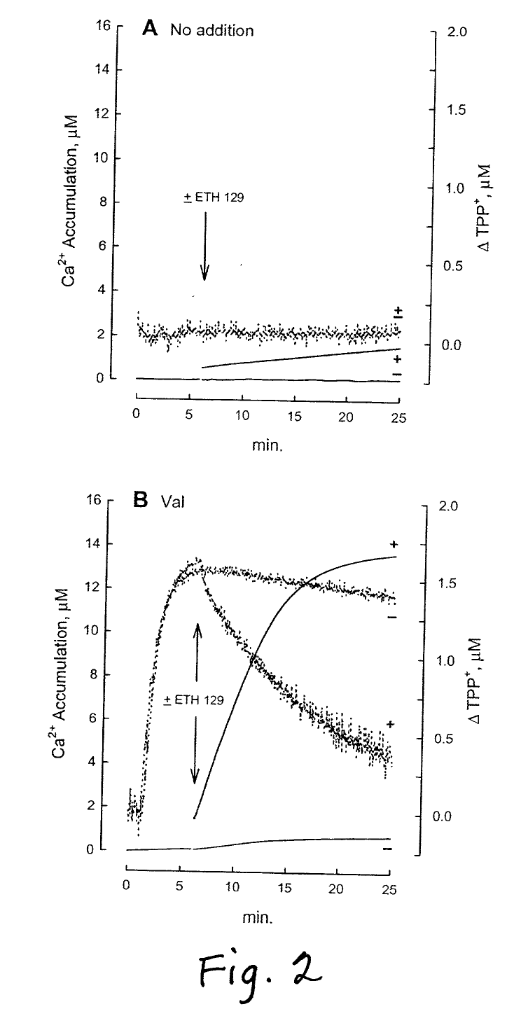

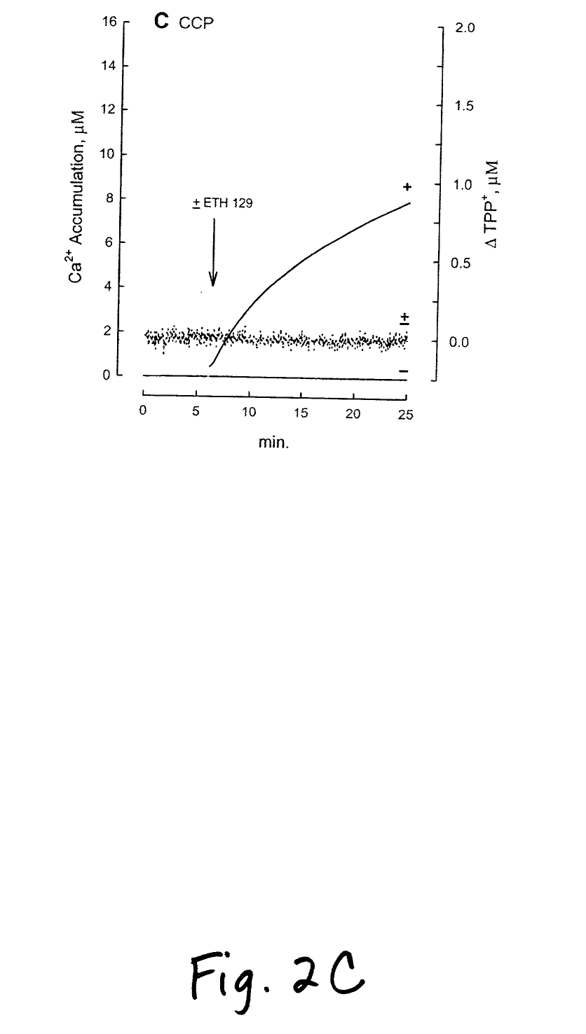

Effect of ΔΨ on transport. To confirm the electrogenic nature of Ca2+ transport mediated by ETH 129, effects of membrane potential on the rate of transport were examined. FIG. 2 shows conditions effecting ETH 129 mediated Ca2+transport. POPC vesicles loaded with Quin-2 and K+ were incubated in the NaCl-Hepes medium, and Ca2+ accumulation (solid lines) and TPP+ accumulation (dotted lines) were monitored, as described above. The nominal concentrations of POPC and Quin-2 were 1.0 mM and 14.2 μM, respectively. For all panels, 100 μM CaCl₂ and 2.0 μM TPP.Cl were present. 5.0 μM ETH 129 was added where indicated to initiate Ca²⁺ accumulation. In FIG. 2A, no further additions were made. In FIG. 2B 0.5 μM Val was present from the beginning of the incubation, and in FIG. 2C 5.0 μM CCP was present, also at the inception of the incubation. TPP+ accumulation indicated formation of a vesicle membrane electrical potential that is oriented inside-negative. Under the conditions employed, accumulation of 1.5 μM TPP+ corresponded to a potential of 187 mV.

Calcium ions permeated the limiting membrane of K+ loaded POPC vesicles very slowly, and the presence of ETH 129 alone had only a modest effect on the rate of that process (FIG. 2A), (Erdahl et al., 1994 Bipohys. J. 66:1678). These observations were expected for an ionophore that acts by an electrogenic mechanism, where a large inside positive membrane potential would arise quickly and oppose further calcium ion translocation. No membrane potential was detected using the TPP+ electrode technique under these conditions (FIG. 2A), however, because of the inside positive orientation of the vesicles. According to non-limiting theory, the modest Ca²⁺ transport activity seen in FIG. 2A was a consequence of an opposing membrane potential, such that the presence of Val or CCP would be expected to collapse this potential and allow divalent cation transport to proceed more rapidly. Such an effect resulted when either Val (FIG. 2B) or CCP (FIG. 2C) was present.

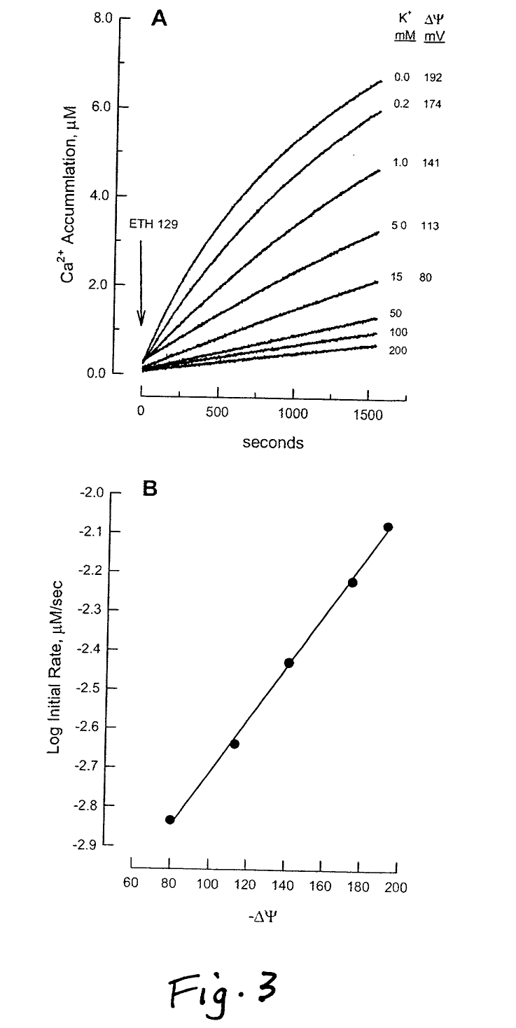

To further characterize the ETH 129 electrogenic mechanism of divalent cation transport, the magnitude of the potential induced by Val was varied and the corresponding effect on Ca2+ transport was determined. FIG. 3 shows the effect of membrane potential on the rate of transport. Vesicles were incubated as described above for FIG. 2, except the concentrations of POPC and CaCl₂ were 1.5 mM and 30 μM, respectively (FIG. 3A). The external Na+ concentration was progressively lowered by equimolar replacement with K+ to reduce the membrane potential formed through the action of Val to desired values (total concentration of Na++K+=200 mM). The external K+ concentrations, and the observed values of the potential that resulted, are shown next to the individual traces. Val was present from the beginning and transport was initiated by the addition of 5.0 μM ETH 129, as indicated. In FIG. 3B; log initial rate values are shown as a function of the induced potential. Potentials less than approximately 60 mV could not be accurately determined under the conditions employed. A linear relationship was seen between the log of the transport rate and ΔΨ, with a slope value of 0.12 μM/sec/mV and a rate at zero potential (extrapolated) of 0.41 nM/sec. The slope value was equivalent to a 3.3-fold acceleration per 59 mV increase in ΔΨ, whereas the rate at zero potential, determined by extrapolation, was slightly lower than the rate seen when CCP was used to eliminate an opposing potential (0.88 nM/sec in FIG. 2 C). Without wishing to be bound by theory, it appears from these data that ΔΨ facilitated ion transport directly, and thus contributed to the rate enhancement produced by Val as described above in FIG. 2.

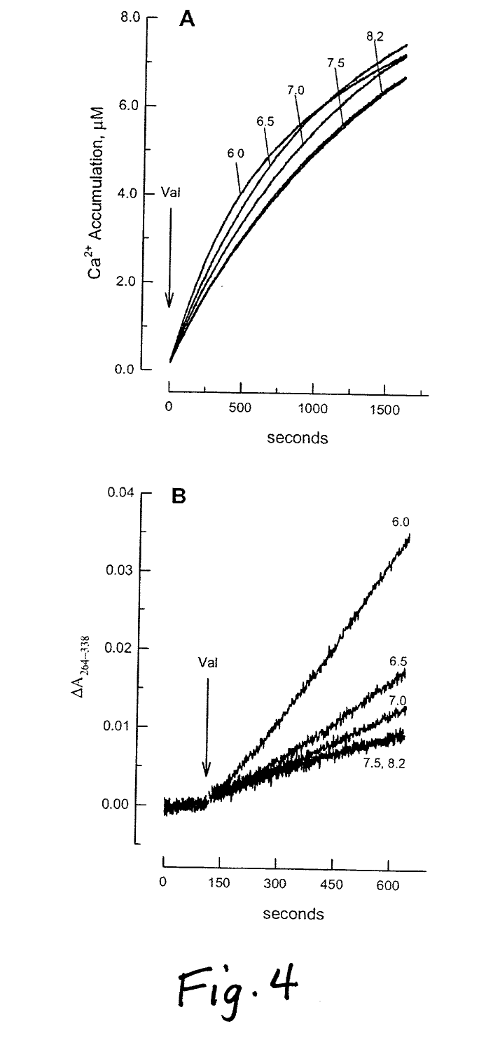

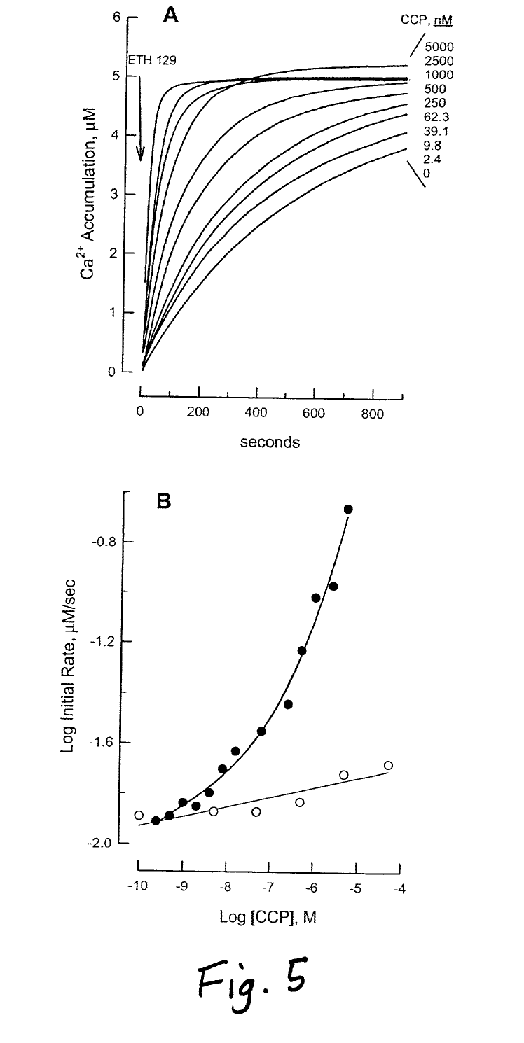

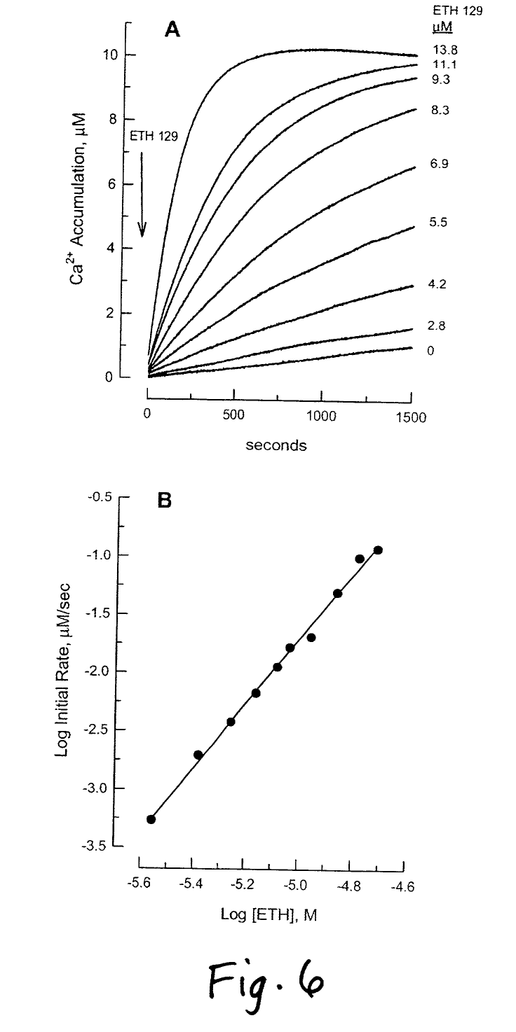

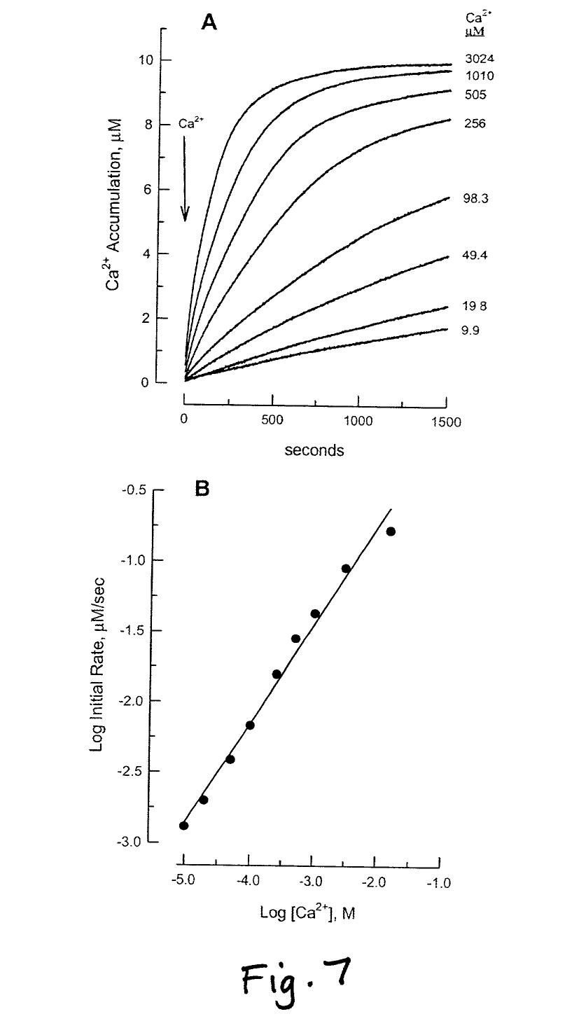

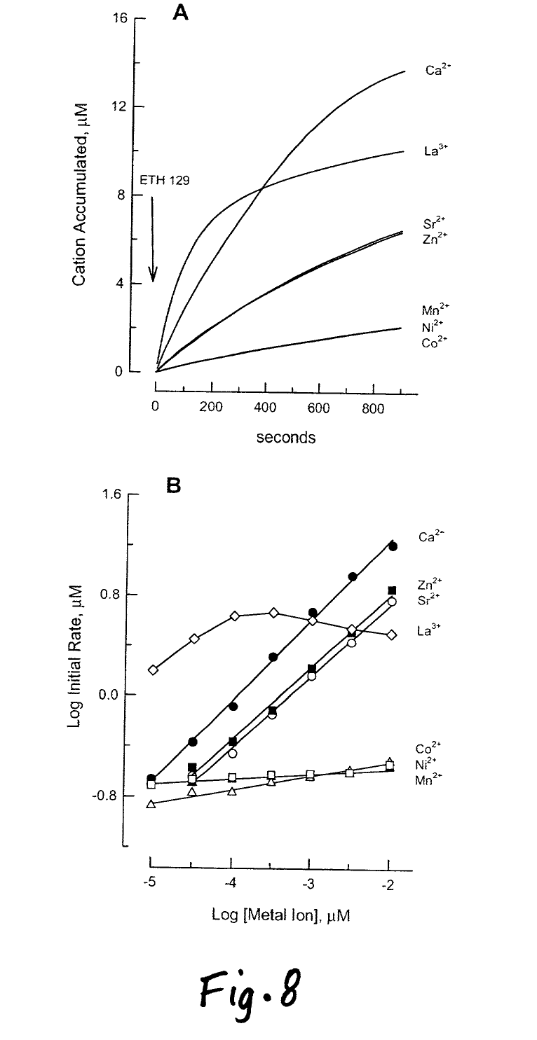

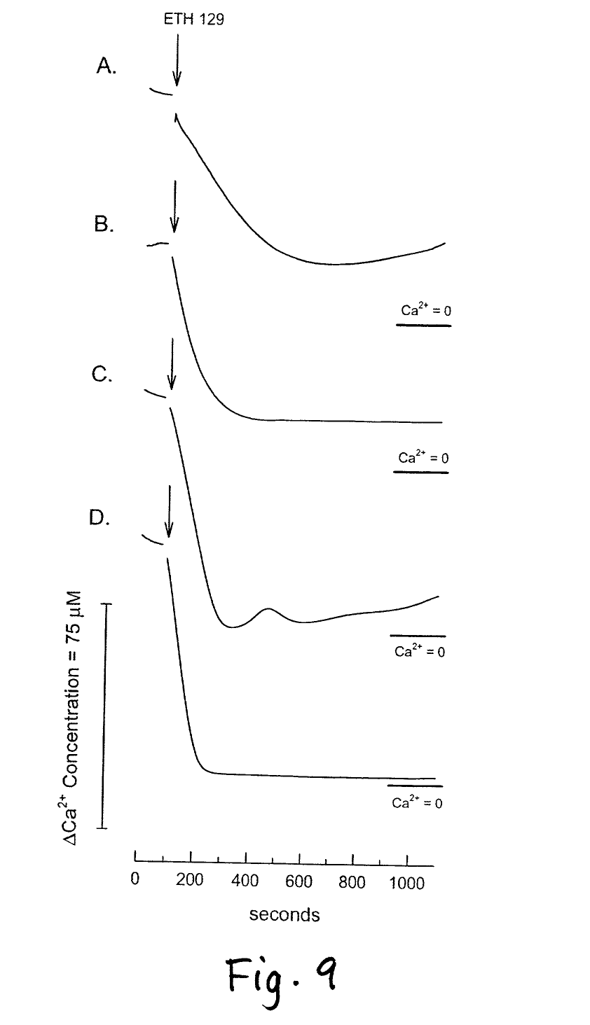

The effects of pH in the presence of a high membrane potential were next examined to further characterize the contribution of the protonophore CCP to the reduced calcium transport rate observed (relative to that determined in the presence of Val) as described above (FIG. 2), when CCP was used to collapse the membrane potential that opposes Ca2+ translocation. FIG. 4 shows the effect of pH conditions on the rate of Ca²⁺ transport. In FIG. 4A, conditions were the same as described above for FIG. 3A, except that (i) the external Na+ concentration was 100 mM and K+ was not added, (ii) 5 mM each of Hepes (N-(2-hydroxyethyl)piperazine-N′-(2-ethanesulfonic acid) and Mes (2-(N-morpholino)ethanesulfonic acid) were employed instead of Hepes alone, and the pH was varied as indicated, and (iii) 5.0 μM ETH 129 was present from the beginning with the time of Val addition designated as 0 seconds. It first appeared that the Ca2+ transport rate decreased as the external pH rose from 6.0 to 7.6, but was not changed in response to a further increase to pH=8.2 (FIG. 4A). However, when ETH 129 and Ca²⁺ were absent, the difference absorbance of entrapped Quin-2 showed time and potential dependent changes that were the same as those reflecting Ca2+ transport, and that had the same dependence on pH (FIG. 4B).

In FIG. 4B; the experimental conditions were analogous to those just described for FIG. 4A, except that Ca²⁺ was absent from the medium. The difference absorbance wavelength pair employed (FIG. 4B, y-axis) was the same one used for monitoring Ca2+ transport, which in this case resulted in increasing AA that reflected an increasingly more acidic vesicle lumen. According to non-limiting theory, these data appear to indicate that uncatalyzed H+ diffusion into the vesicles can occur when AT is high, an event favored by acidic pH. Under these conditions, the internal vesicular volume may become progressively more acidic, the protonation state of entrapped Quin-2 can increase [pKa⁴ of Quin-2=6.25 (Yuchi et al., 1993 Bull. Chem. Soc. Jpn. 66:3377)], and a resulting spectral change occurs that can be mistaken for Ca²⁺ transport. When these spectral changes are subtracted from Ca2+ transport data before they are calibrated, the minor apparent pH dependence seen in FIG. 4A is substantially eliminated (data not shown). Alterations in the pH gradient thus appeared unlikely to influence the rate of Ca²⁺ transport in the presence of CCP.

In addition to varying the pH, activation of Ca2+ transport afforded by CCP was also determined as a function of the CCP concentration. FIG. 5 shows the effect of CCP concentration on the rate of calcium cation transport. For FIG. 5A, conditions were the same as described above for FIG. 3A except that external Na+ concentration was 100 mM and K+ was not added. In addition, the concentration of CaCl₂ was 1.0 mM and a reduced concentration of Quin-2 was used during vesicle preparation when preparing the vesicles (resulting in a reduced content of entrapped Quin-2). Various concentrations of CCP were present from the initiation of the incubation, as indicated in sequence to the right of the individual traces (FIG. 5A). Closed circles (●) in 5B depict log initial rate values from FIG. 5A, expressed as a function of Log CCP concentration. For clarity, some of the traces represented in FIG. 5B were not drawn in FIG. 5A. Open circles in FIG. 5B (∘) represent values obtained from a set of experiments like those shown in FIG. 5A, except that various concentrations of oleic acid (18:1) had been used instead of CCP. A complex dependence was thus observed, with the rate enhancement increasing progressively as the CCP concentration rose to 5 μM. The levels of CCP used in FIG. 5 were in excess of CCP concentrations reported to collapse an opposing membrane potential arising from electrogenic Ca2+ transport at the rates observed (Erdahl et al., 1994 Biophys. J. 66:1678; Erdahl et al., 1995 Biophys. J. 69:2350), giving rise to a non-limiting model whereby CCP may associate with the ETH 129:Ca2+ complex to form a species of reduced charge that crosses the membrane relatively easily. Apparent specificity for this model derives from the inability of oleate (a lipophilic fatty acid anion) to substitute for CCP in this process (FIG. 5B).

The nature of the calcium cation-transporting species, and its cation selectivity, were next explored. To investigate the ionophore:cation stoichiometry of the Ca2+ transporting species, the effects of ETH 129 and Ca2+ concentrations on the rate of transmembrane transport were determined. The rate varied with a regular dependence on both parameters, as shown in FIGS. 6A and 7A. FIG. 6 shows the effect of ionophore (ETH 129) concentration on the rate of Ca²⁺ transport. For FIG. 6A, conditions were the same as described above for FIG. 3A, except the medium concentration of Na+ was 100 mM and K+ was not added. Val was present from the beginning of the incubation, with Ca²⁺ transport initiated by the addition of ETH 129 at the indicated concentration. In FIG. 6B, log initial rate values are shown as a function of log ETH 129 concentration. FIG. 7 shows the effect of Ca2+ concentration on the rate of Ca2+ transport. Assay conditions for FIG. 7A were the same as described above for FIG. 5, except the medium Ca2+ concentration was varied as shown. ETH 129 (5.0 μM) and Val (0.5 μM) were present from the beginning, with transport initiated by the addition of Ca2+ as shown. In FIG. 7B; log initial rate values are shown as a function of log Ca2+ concentration. Plots of log rate vs. log ionophore (FIG. 6B) or log Ca2+ concentration (FIG. 7B) were straight lines that had slope values of 2.8 and 0.7, respectively (FIGS. 6B and 7B). The former value was comparable to that reported by Prestipino et.al. (1993 Anal. Biochem. 210:119), who utilized conductance measurements and planar lipid bilayers to characterize ETH 129 as a Ca2+ ionophore. This value would also be consistent, by non-limiting theory, with a predominant calcium ion-transporting species having 3:1 (ionophore:cation) stoichiometry, which stoichiometry has also been demonstrated using X-ray crystallography (Neupert-Laves et al., 1982 Crystallogr. Spectrosc. Res. 12:287).

Similar experiments conducted using other cations provided the data shown in FIG. 8 and Table 1. FIG. 8 shows the cation specificity of ETH 129 facilitated transport. In FIG. 8A, assay conditions were the same as described above for FIG. 3A, except the medium concentration of Na+ was 100 mM and K+ was not added. 30 PM of the indicated cation chloride and Val were present from the beginning. Transport was initiated by the addition of 5 PM ETH 129, as indicated. FIG. 8B shows log initial rate values as a function of log cation concentration. The values were obtained as described for FIG. 8A except the cation concentrations were varied as indicated. Among the divalent cations, the selectivity sequence was Ca²⁺>Zn^{2+≈Sr}²⁺>Co2+≈Ni²⁺≈Mn²⁺. Two groups were identified within this sequence. Ca²⁺, Zn²⁺ and Sr²⁺ were transported at rates that varied substantially with the cation concentrations. In contrast, the rates of Co²⁺, Ni²⁺ and Mn²⁺ transport were relatively independent of cation concentration within the range examined, as was also the case for La³⁺ transport (FIG. 8B). The trivalent cation La³⁺ was transported more efficiently than divalent cations when the comparisons were made at cation concentrations below 1 mM (FIG. 8).

TABLE 1
Log vs. log plot parameters for the transport
of selected cations by ETH 129^{a}

| Parameter | Sr²⁺ | Ca²⁺ | Mn²⁺ | Co²⁺ | Ni²⁺ | Zn²⁺ | La³⁺ |
|---|---|---|---|---|---|---|---|
| Slope_{ETH 129} | 2.0 | 1.5 | 0.85 | 0.55 | 0.78 | ND | ND |
| Slope_{Me} | 0.57 | 0.65 | 0.04 | 0.11 | 0.05 | 0.58 | nonlin |
| Selectivity, | | | | | | | |
| Ca/Me: | | | | | | | |
| 10^{-2.0} M | 2.8 | ≡1.0 | 59 | 53 | 55 | 2.3 | 5.1 |
| Cations | | | | | | | |
| 10^{-3 5} M | 2.9 | ≡1.0 | 8.9 | 9.9 | 8.6 | 2.6 | 0.44 |
| Cations | | | | | | | |
| 10^{-5.0} M | 1.0 | ≡1.0 | 1.1 | 1.6 | 1.1 | 0.9 | 0.14 |
| Cations | | | | | | | |

^{a}Slope_{Me} values were obtained from the data shown in FIG. 8B. Selectivity values are from the same data and represent the rate of Ca²⁺ transport divided by the rate for the cation of interest when both cations were present at the indicated concentration. Slope_{ETH 129} values were obtained from experiments like those shown in FIG. 6, except that the cation concentration employed was 1.0 mM.

### Example 2

### Mitochondrial Calcium Uptake in Saccharomyces Cerevisiae Exposed to Eth 129, an Electrogenic Calcium Ionophore

This example describes facilitation of Ca²⁺ uptake in yeast mitochondria by the electrogenic divalent cation ionophore ETH 129, which was described in Example 1. Calcium uptake in yeast as described herein may proceed without induction of MPT. The mitochondria of certain yeast species do not contain a calcium uniporter (Balcavage et al., 1973 Biochim. Biophys. Acta 305:41; Jung et al., 1997 J. Biol. Chem. 272:21104; Manon et al., 1993 J. Bioenerg. Biomemb. 25:671) and are thought to lack high activity calcium release carriers that oppose the uniporter in other mitochondria (Weihua et al., 1992 J. Biol. Chem. 267:17983). Yeast mitochondria do feature general diffusion properties in the inner membrane, and are subject to a permeability transition, although Ca²+ does not regulate this transition as it does in mammalian mitochondria (Jung et al., 1997). In addition, the production of reducing equivalents and ATP synthesis do not appear to be regulated by Ca2+ in yeast mitochondria (Nichols et al., 1994 Biochem. J. 303:461).

Preparation of Yeast Mitochondria.

The yeast strain W303-1A (Saccharomyces cerevisiae) was grown aerobically at 30° C. in a medium containing 2% lactate, 1% yeast extract, 2% peptone, 0.05% dextrose, and 0.01% adenine at pH 5.0, and cells were harvested during the logarithmic phase (A600=1.8−2.2). Mitochondria were isolated from spheroplasts as previously described (Jung et al., 1997 J. Biol. Chem. 272:21104), except that 0.6 M sucrose was used in the homogenization medium instead of 0.6 M mannitol (Daum et al., 1982 J. Biol. Chem. 257:13028). The resulting preparations were maintained on ice and were suspended in 0.6 M mannitol 20 mM HEPES (K+) (pH 6.8), containing 0.75 mg/ml BSA and 0.1 mM EGTA. Protein concentration was determined by a reduced volume Biuret method in which 50 μl of the final preparation were solubilized by adding an equal volume of 10% deoxycholate (Na+), with 0.4 ml of Biuret reagent added to the resulting mixture. BSA was employed as the standard.

Determination of Ion Transport, ΔΨ and Related Parameters.

Unless otherwise noted, mitochondria were incubated at 1 mg protein/ml and at approximately 25° C. in a medium that contained 0.6 M mannitol, 10 mM Pi (K+), 10 mM Hepes (TEA+), pH 7.2, plus 1 mM ethanol as an oxidizable substrate. Compounds having a low solubility in water were added from stock solutions prepared in methanol or DMSO. Ca2+ transport and membrane potential were monitored using the indicating dyes antipyrylazo III (ΔA₇₂₀₋₇₉₀) and safranin O (ΔA₅₁₁₋₅₃₃), respectively (Akerman et al., 1976 FEBS Lett. 68:191; Scarpa et al., 1978 Biochem. 17:1378). An SLM Aminco DW-2C spectrophotometer operated in the dual wavelength mode was employed for these purposes. The same instrument, operated in split beam mode, was used to monitor mitochondrial swelling as a decrease in apparent absorbance of mitochondrial suspensions at 540 nm. Oxygen consumption data were obtained using a Clark-type electrode in parallel experiments. Atomic absorption spectroscopy was utilized to monitor the transport of other cations by yeast mitochondria. Briefly, samples were taken at appropriate times and the mitochondria were rapidly sedimented in a microcentrifuge. After carefully removing supernatants, the pellets were solubilized with 2 N percholoric acid (overnight) and centrifuged a second time. The resulting supernatants were diluted with an appropriate volume of distilled water and the concentration of the cation in question was subsequently determined.

Determination of Free Fatty Acids and Mitochondrial Phospholipids.

Samples containing 3 mg of mitochondrial protein were extracted by a modified Folch technique (Broekemeier et al., 1985 J. Biol. Chem. 260:105), following the addition of a known amount of heptadecanoic acid (17:0) which was employed as an internal standard (Pfeiffer et al., 1979 J. Biol. Chem. 254:11485). The lipid-containing phase was reacted with diazomethane to produce fatty acid methyl esters (Schlenk et al., 1960 Anal. Chem. 32:1412) and silica gel mini-columns were used to separate these from other fractions (Broekemeier et al., 1985). The fatty acid methyl esters were quantified by gas-liquid chromatography, using an instrument equipped with a capillary column and a computing integrator (Broekemeier et al., 1985; Pfeiffer et al., 1979). Peak areas were converted to units of nmol/mg of mitochondrial protein by considering the internal standard peak area and the amount of protein represented by the sample. Total mitochondrial phospholipids were estimated from measurements of lipid phosphorous in an aliquot of the initial extract (Bartlett, 1959 J. Biol. Chem. 234:466).

Highly variable efficiency of ETH 129 mediated Ca2+ transport was observed in yeast mitochondria in terms of the rate of transport, the external Ca2+ concentration that is attained, and the tendency of previously accumulated Ca2+ to be released during an extended incubation (FIG. 9). Conditions for all traces in FIG. 9 were the same except that different preparations of mitochondria were employed. The mitochondria were incubated at 1.0 mg protein per ml and at 25° C. in a medium that contained 0.6 M mannitol, 10 mM HEPES (TEA+, pH 7.2), 10 mM Pi (K+), 0.5 mM ethanol, 0.5 mg/ml BSA, 0.1 mM antipyrylazo, and 80 μM CaCl₂. Where indicated, 2.4 μM ETH 129 was added to initiate Ca2+ accumulation, which was monitored via the antipyrylazo III signal as described above. A downward deflection indicated Ca2+ accumulation, and the heavy bar associated with each trace shows the signal at an external Ca2+ concentration of approximately zero.

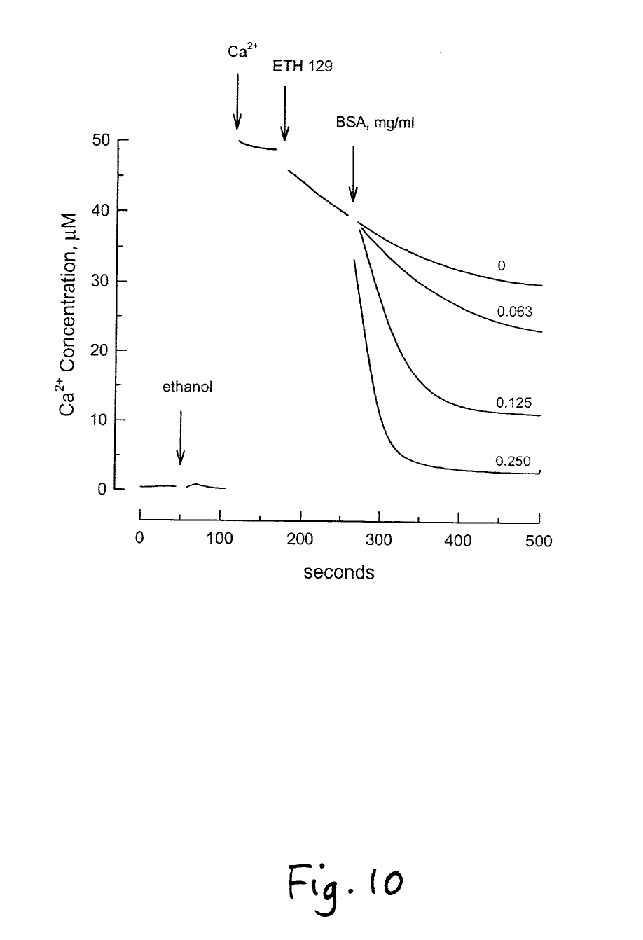

BSA stimulation of ETH 129 mediated Ca2+ transport is shown in FIG. 10. Mitochondria were incubated at 1.0 mg protein per ml and at 25° C. The medium contained 0.6 M mannitol, 10 mM HEPES (tetraethylammonium cation, (TEA+), pH 7.2), 10 mM Pi (K+), 0.1 mM antipyrylazo III, 12 μM safranine O, and 10 μM EGTA (TEA+). Additions of 1 mM ethanol, 60 μM CaCl2, and 3.6 μM ETH 129 were made as indicated in the figure, and Ca2+ accumulation was monitored via the antipyrylazo III signal as described above. A downward deflection indicated Ca2+ accumulation. When utilized, BSA was added at 0.0625, 0.125, or 0.25 mg/ml, as indicated by the number associated with the individual traces (FIG. 10). Calcium accumulation by freshly prepared yeast mitochondria was slow and limited when the medium did not contain BSA. The addition of BSA after the initiation of Ca2+ transport improved both the rate and extent parameters in a concentration-dependent manner. As shown in FIG. 10, a maximal effect was seen at a BSA concentration near 0.25 mg/ml (˜4 μM). BSA was similarly effective when present at the initiation of incubations, while other high molecular weight polymers (10 kDa PEG, 100 kDa dextran) known to have similar effects on medium collide osmotic properties were ineffective (data not shown).

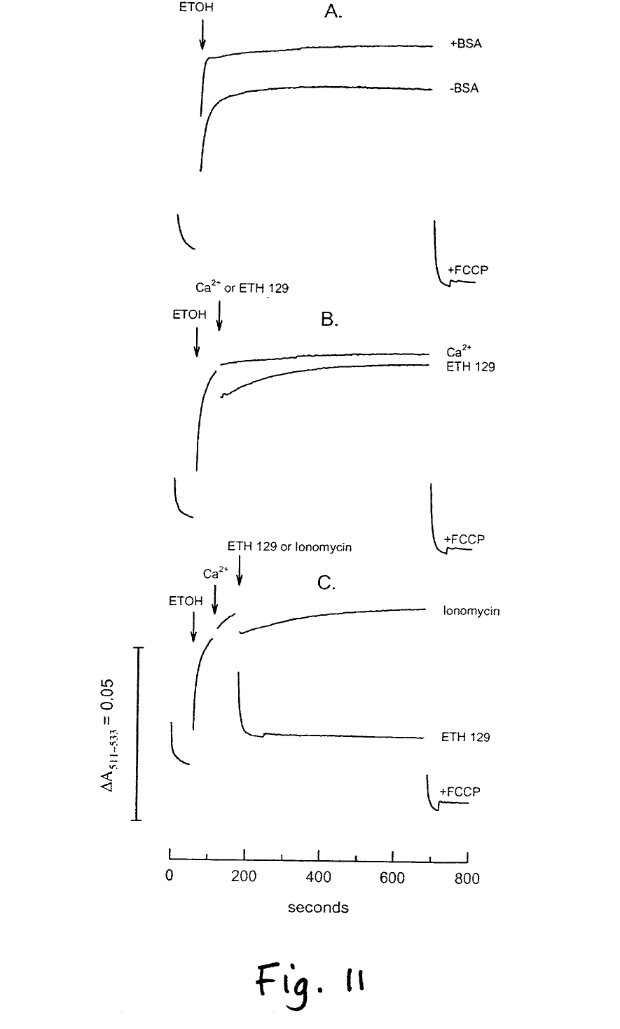

FIG. 11 shows the effects of ETH 129 and Ca2+ on ΔΨ. Mitochondria were incubated as described above with regard to FIG. 10, except that antipyrylazo III was not present. For all traces, 1 mM ethanol was added where indicated and ΔΨ was monitored via the safranine signal as described above. An upward deflection indicated increasing ΔΨ. Also for all traces, 4 μM of the uncoupler trifluoromethylcarbonyl-cyanide phenyl-hydrazone (FCCP) was added at ˜700 seconds to determine the signal at ΔΨ=0. In FIG. 11A, 0.5 mg/ml of BSA was present or absent from the beginning of the incubation, as indicated. In FIG. 11B, BSA was absent for both μM CaCl₂ or 3.6 μM ETH 129 was added as indicated. Conditions in FIG. 11C were the same as in FIG. 11B except both CaCl₂ and an ionophore (ETH 129 or ionomycin) were added as indicated. These mitochondria developed and maintained a large ΔΨ during ethanol oxidation when BSA was absent, although BSA increased the magnitude to some degree (FIG. 11A). The addition of Ca2+ or ETH 129 alone had little effect on ΔΨ (FIG. 11B), however both agents together produced a sustained reduction (FIG. 11 C). This reduction persisted for more time than would be required to complete Ca2+ uptake if BSA were present, based on the results described above (FIG. 10), and such reduced mitochondrial membrane potential was not duplicated when ionomycin instead of ETH 129 was the ionophore added with Ca2+ (FIG. 11C). In this regard, FIG. 11C shows that the simple presence of Ca2+ in the mitochondrial matrix space did not effect ΔΨ. Rather, because ionomycin is an electroneutral ionophore for Ca2+ that equilibrates Ca2+ and H+ gradients across membranes (Erdahl et al., 1994 Biophys. J. 66:1678; Thomas et al., 1997 Arch. Biochem. Biophys. 342:351), these results suggest that an electrogenic mechanism is required for Ca2+ entry into mitochondria under these conditions.

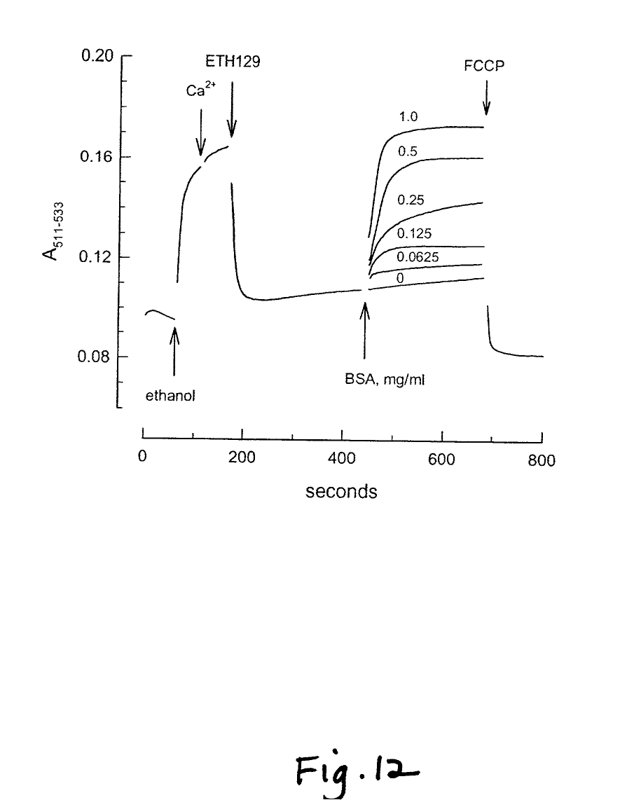

The sustained reduction of ΔΨ produced by simultaneous exposure of mitochondria to ETH 129 plus Ca²⁺ was reversed by the subsequent addition of BSA (FIG. 12). In FIG. 12, conditions were the same as described above for FIG. 1C, with ETH 129 utilized instead of ionomycin. ΔΨ was monitored via the safranine signal as described above, and the upward deflection of the individual traces indicated an increasing value. BSA was added at 0.0625, 0.125, 0.25, 0.50, or 1.0 mg/ml, as indicated by the number associated with the individual traces. As was the case with enhanced Ca²⁺ accumulation (FIG. 10), the degree of reversal appeared to be a function of the BSA concentration, such that a direct correlation was apparent between the actions of BSA on AT and Ca²⁺ transport. When BSA was absent and transport was minimal, ΔΨ was low. Conversely, the efficient transport seen in the presence of BSA was associated with a large ΔΨ.

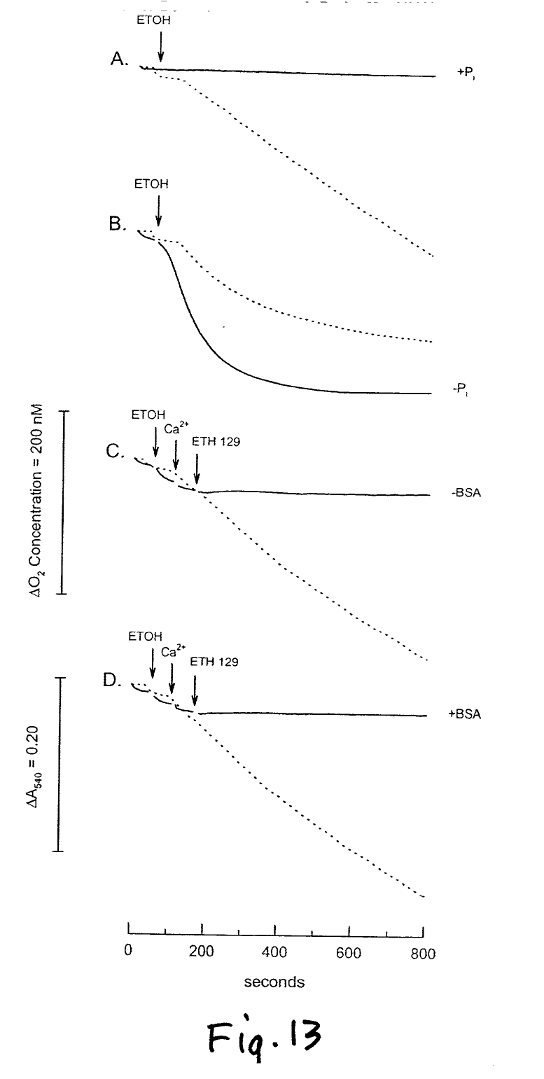

Additional data pertaining to the sustained reduction of AT are shown in FIG. 13, which depicts effects of ETH 129 and Ca2+ on mitochondrial swelling and respiration. In FIG. 13A mitochondria were incubated at 1.0 mg protein per ml and at 25° C. The medium contained 0.6 M mannitol, 10 mM HEPES (TEA+, pH 7.2), 10 mM Pi (K+), and 10 μM EGTA (TEA+). 1 mM ethanol was added where indicated while swelling (solid line) and oxygen consumption (dotted line) were monitored as described above. The conditions in FIGS. 13B-D were the same as in FIG. 13A except that in FIG. 13B the medium did not contain P_{I}; in FIG. 13C 60 μM CaCl₂ and 3.6 μM ETH 129 were added where shown; and in FIG. 13D the medium contained BSA at 0.5 mg/ml. The rate of oxygen consumption in panel A was 34.8 ng atoms O/min/mg protein. The reduction of ΔΨ was apparently unrelated to the yeast mitochondrial permeability transition given that the large amplitude swelling that accompanied the transition induced by respiration in the absence of phosphate (e.g., FIGS. 13A vs. 13B) was not seen when ΔΨ was depressed by ETH 129 plus Ca²⁺ (FIG. 13C). Furthermore, there was little effect of BSA on respiration when ETH 129 and Ca²⁺ were present, (FIGS. 13C vs. 13D), and in neither case did respiration become inhibited as it did when the transition occurred (FIG. 13A and B).

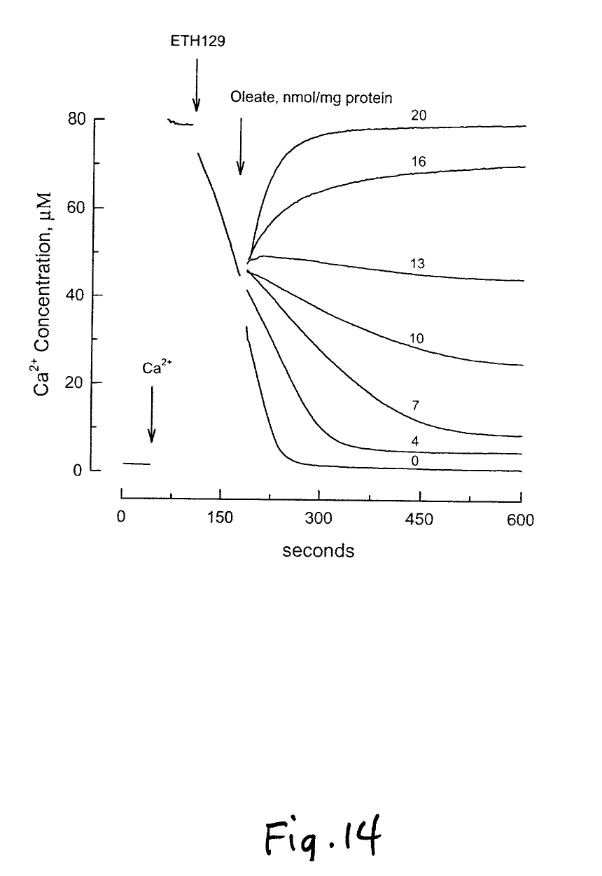

To determine if the fatty acid binding activity of BSA for fatty acids or other hydrophobic molecules (Elmadhoun et al., 1998 Gastrointest. Liver Physiol. 275:G638-G644; Nissani et al., 1983 Arch. Biochem. Biophys. 226:357) contributed to BSA effects on ETH 129 mediated Ca²⁺ transport, the actions of exogenous oleate (18:1) on transport were examined. Mitochondria were incubated and Ca²⁺ transport was monitored as described above for FIG. 10, with 1 mM ethanol and 0.5 mg/ml of BSA present at 0 seconds (FIG. 14). 60 μM CaCl₂ and 3.6 μM ETH 129 were added where indicated, followed by an addition of oleate (Na⁺) dissolved in dimethylsulfoxide. The number associated with the individual traces in FIG. 14 shows the amount of oleate added in units of nmol/mg protein. With BSA present initially at 0.5 mg/ml, the addition of increasing oleate levels during Ca2+ accumulation first inhibited further accumulation and then caused a reversal of the transport that had already occurred (FIG. 14). Although fatty acids uncouple yeast mitochondria through a mechanism that involves participation of the adenine nucleotide translocase (Polcic et al., 1997 FEBS Lett. 412:207), the Ca²⁺ releasing activity of fatty acids, and the opposing effects of BSA, did not merely reflect uncoupling by such a mechanism, since there was no effect of carboxyatractyloside (Brandolin et al., 1993 Biochem. Biophys. Res. Commun. 192:143) on the depression of ΔΨ produced by ETH 129 plus Ca²⁺ when BSA was absent.

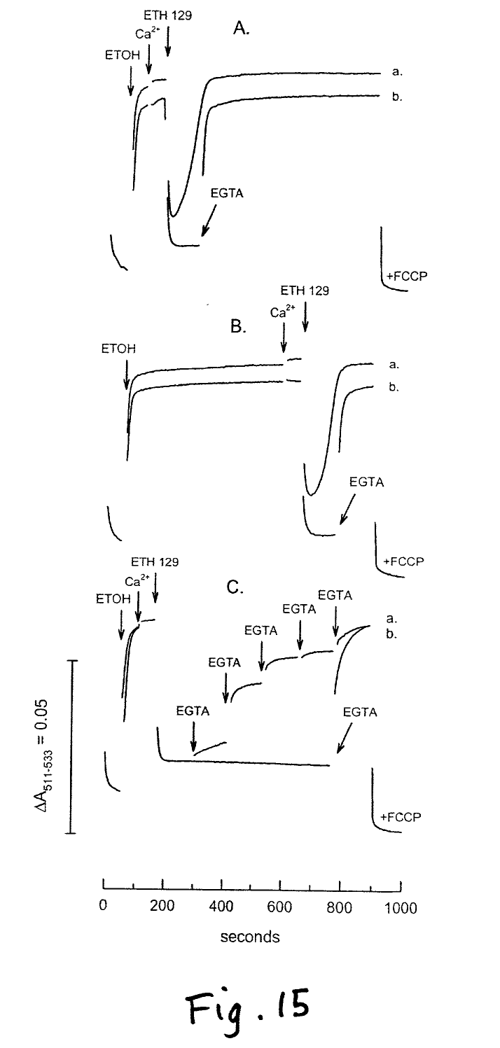

The depression of ΔΨ was reversed when EGTA was added to the external medium, regardless of whether Ca2+ transport occurred early or late during incubation (FIGS. 15A and B), or whether ΔΨ was suppressed briefly or for an extended period before EGTA was added (FIGS. 15A and C). Mitochondria were incubated and ΔΨ was monitored as described above for FIG. 11. For all panels and traces, additions of ethanol (1 mM) and CaCl₂ (60 μM) and ETH 129 (3.6 μM) were as shown in the figure. 4 μM of the uncoupler FCCP was added at ˜900 seconds in all cases to determine the signal at ΔΨ=0. In FIG. 15A, trace a, the medium contained 0.5 mg/ml of BSA from 0 seconds; in trace b, BSA was not present and 2 mM EDTA (TEA⁺) was added where indicated. In FIG. 15B, conditions were the same as panel A except the addition times were altered as shown, to demonstrate that the results were independent of the time of addition. FIG. 15C, trace a, was generated under the same conditions as FIG. 15A, trace b, except that EGTA was added incrementally as shown. The first four additions were of 30 μM EGTA, whereas the final addition was 2 mM. Conditions in FIG. 15C, trace b were the same as FIG. 15 A, trace b except that addition of 2 mM EGTA was delayed as shown, again to demonstrate that results did not depend on the time of addition. In addition, there was a graded response to multiple additions of EGTA when the concentration arising from a single addition was less than the total concentration of Ca²⁺ (FIG. 15C).

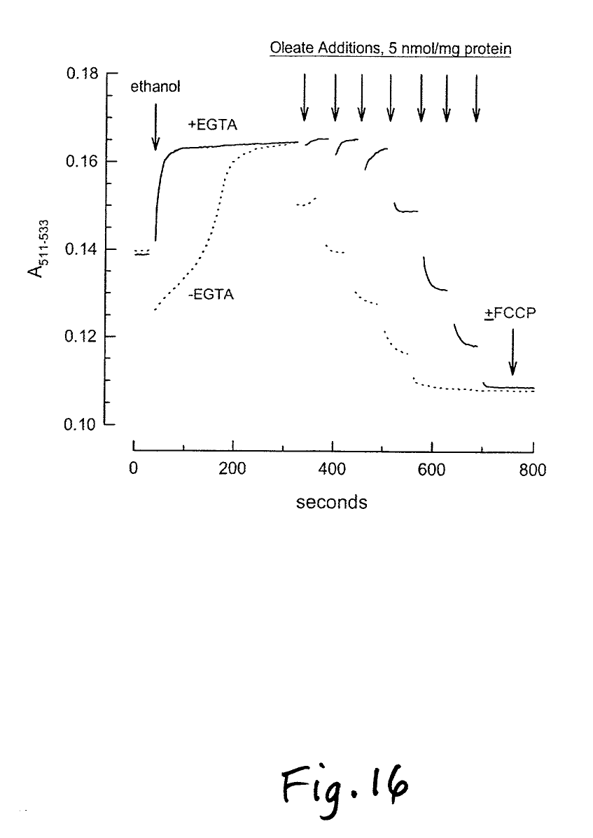

As shown in FIG. 16, oleate differentially effected ΔΨ in the presence and absence of EGTA. Mitochondria were incubated and ΔΨ was monitored as described above for FIG. 11, except that CaCl₂, ETH 129, and BSA were present from the beginning of the incubations. For the data shown in FIG. 16 as solid lines, 2 mM EGTA (TEA⁺) was also present from the beginning, whereas EGTA was not present for the data shown as dotted lines. 1 mM ethanol was added at 0 seconds, followed by repetitive additions of 5 nmol/mg protein of oleate (Na⁺) as shown. When utilized FCCP was added at 4 μM.

Suppression of ΔΨ began immediately during titration with exogenous oleate when ETH 129, free Ca²⁺ and BSA were available (FIG. 16, dotted lines). This result contrasted with the titration preformed in the presence of excess EGTA where free Ca²⁺ was not available, but fatty acid dependant uncoupling could still occur. Under those conditions, exogenous oleate did not depress ΔΨ until the level added exceeded 15 nmol/mg protein. Levels of fatty acids that correlated with extensive uncoupling thus appeared to be higher than those that accompanied the decrease in AT detected in the presence of free Ca²⁺ and ETH 129.

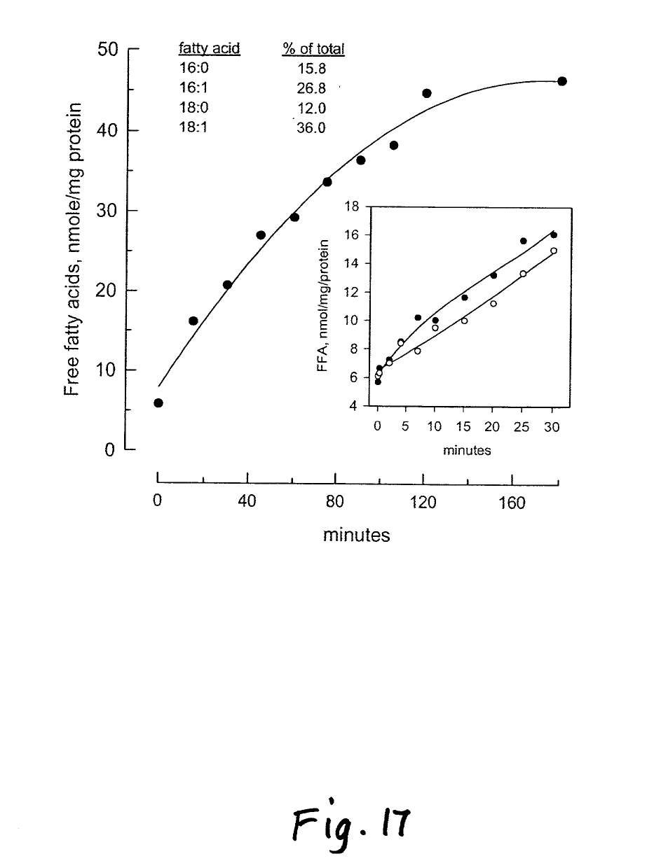

Free fatty acids accumulated in isolated yeast mitochondria at levels sufficient to alter Ca²⁺ transport, and were determined as a function of incubation time FIG. 17. Mitochondria were incubated in 0.6 M mannitol, 10 mM HEPES (TEA⁺, pH 7.2), 10 mM P_{I} (K⁺), 100 μM EGTA (TEA⁺), and 1 mM ethanol. Samples were taken periodically for the determination of free fatty acids as described above. FIG. 17 shows the composition (“% of total”) of free fatty acids that were present in the sample taken at 170 minutes. Large amounts of free fatty acids accumulated in mitochondria during incubation at 25° C., at an initial rate of approximately of 0.4 nmol/min/mg protein (FIG. 17). Using a shortened time-course and otherwise identical conditions except that ethanol was present (●) or absent (∘), this rate was not altered substantially by the presence or absence of ethanol (FIG. 17 insert), by free Ca²⁺ and ETH 129, or by the presence of BSA. However, the rate was decreased to approximately 0.3 nmol/hr/mg protein when the mitochondria were maintained on ice (not shown).

16:0, 16:1, 18:0, and 18:1 accounted for ˜90% of the accumulating fatty acids (FIG. 17). These compounds and their relative proportions were typical of the fatty acid composition found in yeast phospholipids (Daum et al., 1998 Yeast 14:1471). Parallel determinations of lipid phosphorus showed that the phospholipid content of the preparations decreased at approximately 50% of the rate at which the fatty acids accumulate in total (not shown). Furthermore, there was little change in the rate or composition parameters when Percoll gradient purified mitochondria (Broekemeier et al., 1991 J. Biol. Chem. 266:20700) were employed instead of the standard preparation. Accordingly, the free fatty acids that accumulated appeared to arise from phospholipase-catalyzed hydrolysis of mitochondrial phospholipids, and that both sn-1 and sn-2 positions were hydrolyzed by the degradative activities.

To investigate cation selectivity of the mitochondrial Ca²⁺ release activity, mitochondria were also incubated as described above, with 0.5 mg/ml of BSA and 20 μM oleate present at 0 seconds. 1 mM ethanol, 80 μM of a cation chloride (Sr²+ or Mg²⁺), 3.6 μM ETH 129, and 4 μM FCCP were then added. A 1 ml aliquot was taken from the 3 ml volume in the cuvette, and the mitochondria were rapidly sedimented in a microcentrifuge. The distribution of the exogenous cation between the supernatant and the pellet was then determined by atomic absorption spectroscopy. About 85% of the endogenous Mg²⁺ was retained in all cases.

### Example 3

### CA²⁺ Transport in Mitochondria from Yeast Expressing Recombinant Aequorin

This example describes aequorin expression in the mitochondria of yeast (Saccharomyces cerevisiae) and use of the resulting recombinant yeast strain to investigate mitochondrial Ca²⁺ transport in vivo and in vitro. The expression of mitochondrial Ca²⁺ transporters in yeast appears to be an attractive strategy for pursing molecular studies because yeast mitochondria lack analogous activities (Carafoli et al., 1970 Biochim. Biophys. Acta 205:18; Manon et al., 1993 J. Bioenerg. Biomemb. 25:671; Jung et al., 1997 J. Biol. Chem. 272:21104; Balcabage et al., 1973 Biochim. Biophys. Acta 305:41; Gunter et al., 1990 Am. J. Physiol. 258:C755; Hansford, 1994 J. Bioenerg. Biomembr. 26:495; Uribe et al., 1992 Cell Calcium 13:211). For example, unlike mammalian mitochondria, mitochondria of Saccharomyces cerevisiae accumulate large amounts of Ca²⁺ when oxidizing ethanol, if the medium contains a high phosphate concentration and an electrogenic ionophore for Ca²⁺ (e.g., ETH 129; Jung et al., 1997). Additionally, such yeast mitochondria are not subject to a Ca²⁺ and phosphate dependent permeability transition, even at Ca²⁺ loads of ≧400 nmol/mg protein (Jung et al., 1997). Moreover, yeast mitochondria are known to express, import, and process other mitochondrial proteins from mammalian sources (Scarpulla et al., 1986 Proc. Nat. Acad. Sci. USA 83:6352; Murdza-Inglis et al., 1991 J. Biol. Chem. 266:11871; Koshy et al., 1992 Prot. Express. Purific. 3:441); and extensive information about the yeast genome is available. Targeted expression in yeast mitochondria of the divalent cation-sensitive indicator molecule aequorin is described herein, as an example of a useful host cell according to the present invention.

Plasmid Construction and Transfection.

The mitochondrial-targeting apoaequorin expression vector for mammalian cells, mtAEQ/pMT2, developed by Rizzuto et al. (1995 Meths. Enzymol. 260:417), was obtained from Molecular Probes, Inc. (Eugene, Oreg.). The cDNA insert in this vector is flanked by EcoR1 sites and contains the apoaequorin structural gene fused to a sequence encoding the N-terminal targeting region from subunit VIII of human cytochrome c oxidase. This insert was subcloned into the multiple cloning site of the pYES2 yeast expression vector (Invitrogen Corp., San Diego, Calif.) using EcoR1. The pYES2 plasmid contains the Gall portion of the Gal1/Gal10 promoter region from Saccharomyces cerevisiae for inducible expression, and also contains the URA3 gene for selection. Vector propagation was performed by transformation into competent E. coli cells. The ampicillin-resistant transformants were grown and the plasmids were purified by standard techniques (Sambrook et al., 1989 Molecular Cloning: A Laboratory Manual, Cold Spring Harbor Laboratory Press, Cold Spring Harbor, N.Y.). Orientation and insert number were checked by agarose gel electrophoresis of restriction enzyme digests.

Another plasmid, designated ymtAeq/pGK, was also constructed. This plasmid directed the expression of a mitochondrially targeted aequorin fusion protein using a yeast mitochondrial cytochrome c oxidase subunit IV (COX IV) mitochondrial targeting polypeptide region (Hurt et al., 1985 EMBO J. 4:2061) instead of the human cytochrome c oxidase subunit VIII N-terminal targeting region that was used in the pYES2 construct described above. Aequorin-encoding cDNA, lacking the human mitochondrial targeting sequence and including the first twelve amino acids of the yeast mitochondrial targeting polypeptide (Hurt et al., 1985), was amplified by PCR using the pYES2 mitochondrially targeted aequorin construct as template and the following oligonucleotide primers:

| | |
|---|---|
| Forward oligo: | |
| 5′-AAAAGATCTAAAAATGCTTTCACTACGTCAATCTATAAGATTTTTCAAGCT | SEQ ID NO:8 |
| TACATCAGACTTCGACAACCC—3′ | |
| Reverse oligo: | |
| 3′-AAAGGTACCTTAGGGGACAGCTCCACCGT-3′ | SEQ ID NO:9 |

Coding sequence for the first twelve amino acids of the mitochondrial targeting sequence from yeast COX IV, and restriction endonuclease recognition sites, were incorporated into the forward oligonucleotide (SEQ ID NO:8) as presented above.

The PCR amplification product was then cloned into the pGK yeast expression vector (Brunelli et al., 1993 Yeast 9:1299-1308) obtained from Drs. J. P. Brunelli and M. L Pall (Department of Genetics and Cell Biology, Washington State University, Pullman, Wash.) using the Bg1 II and Asp718 I cloning sites in the pGK vector. This construct was then used to transform yeast strain INVScl (Invitrogen Corp., San Diego, Calif.) according to the supplier's recommendations.

Yeast cells (strain X4003-5B; leu2, ade1, his4, met2, ura3, trp5, gal1) obtained from the Yeast Genetic Stock Center (Berkeley, Calif.) were transformed with the pYES2 plasmid, encoding the human mitochondrially targeted apoaequorin fragment, using the lithium acetate method (Sambrook et al., 1989), and INVSc1 cells were transformed with the pGK plasmid encoding the yeast mitochondrially targeted apoaequorin fragment. Selection was carried out on plates prepared with synthetic complete medium minus uracil (SC-U) (Sherman, 1991 Meths. Enzymol. 194:3). Transformed colonies were cultured in a small volume of SC-U and assayed for aequorin activity in vitro as described by Nakajima et al. (1991 Proc. Nat. Acad. Sci. USA 88:6878). Briefly, the cells were harvested by centrifugation, resuspended in lysis buffer (50 mM Tris, 2 mM EDTA, 0.5 mM PMSF, pH 8 w/NaOH), disrupted by vortexing with glass beads, and then treated with 0.1% Triton X-100 to release apoaequorin from the mitochondria. Following centrifugation, 200 μl aliquots of the supernatants were incubated with coelenterazine (2.5 μM) and 2-mercaptoethanol (2 μl), for ˜3 hr while standing on ice, to form the luminescent aequorin from its apoprotein. The coelenterazine cofactor is highly hydrophobic and readily permeates yeast cell membranes (Nakajima et al., 1991) such that reconstitution was conducted without disrupting the cells in some cases. 2-mercaptoethanol was omitted when intact cells were employed. A transformant which yielded high luminescence (named X4003-5B-AEQmito1) was selected for further characterization.

Cell Culture; Isolation and Incubation of Yeast Mitochondria.

For preculture, the transformant was inoculated into 50 ml of SC-U plus 2% glucose, and incubated in an air shaker at 180 rpm and 30° C. At 24 hr, the preculture was transferred to 500 ml of a medium containing 1% yeast extract, 2% peptone, 2% lactate, 1% galactose, 0.012% adenine, pH 5 w/NaOH, and growth was continued under conditions which were otherwise the same. Cells were harvested after an additional 24 hr, spheroplasts were prepared using zymolyase, and mitochondria were isolated by the method of Daum et al. (1982) as previously described (Jung et al., 1997). The final pellet was resuspended at ˜20 mg protein/ml in 0.6 M mannitol, 20 mM HEPES (K⁺ salt, pH 6.8). Apoaequorin in the intact or disrupted mitochondria was reconstituted by incubation with coelenterazine as described above for cells.

For most experiments, the mitochondria were present at 0.4 mg protein/ml in a standard medium that contained 0.3 M KCl, 20 mM HEPES, (K⁺ salt, pH 7.10), 10 mM KH₂PO₄, 2 mM EGTA, and 2 mM MgCl₂. Other additions are described in greater detail below. Ca²⁺ buffers were formed by adding various amounts of CaCl₂ to this EGTA containing medium. The free Ca²⁺ concentrations which resulted were calculated using a computer program (Brooks et al., 1992 Anal. Biochem. 201:119).

Measurement and Calibration of Luminescence as a Function of Ca²⁺ Concentration.

Luminescence measurements were carried out in 6×50 mm culture tubes, using a total volume of 0.3 ml, and were started by injection of 5-20 μl of mitochondria or cells. A SAI model 3000 integrating photometer was employed, with AJD sampling at 0.4 seconds intervals driven by Asyst software. Values were transformed into corresponding Ca²⁺ concentrations by computer assisted calculations based upon published calibration methods (Allen et al., 1977 Science 195:996; Brini et al., 1995 J. Biol. Chem. 270:9896). For each value, the ratio of observed luminescence (L) to the potential luminescence remaining (Lₘₐₓ) was calculated, and compared to a calibration curve which relates this ratio to the free Ca²⁺ concentration. Thus, complete calibrated progress curves were generated. The total luminescence available initially (Lₜₒₜₐₗ) must be known and was determined in separate experiments, wherein a saturating Ca²⁺ concentration (20 μM) is employed to discharge the aequorin completely. This value allows Lₘₐₓ to be calculated at any time, as Lₜₒₜₐₗ minus the sum of luminescence that already occurred.

The calibration data were obtained at an ionic strength of 335 mM and in the presence of 1 mM Mg²⁺, to mimic conditions in the mitochondrial matrix, because both factors influence the affinity of aequorin for Ca2+ (Cobbold et al., 1987 Biochem. J. 248:313). Luminescence values were corrected for low background values that are obtained when Ca²⁺ is absent before conducting further calculations.

To relate the free Ca²⁺ concentration determined by luminance to the total content of Ca²⁺ in mitochondria, samples were taken periodically from a larger incubation and mitochondria were rapidly sedimented in a microcentrifuge. After carefully removing the supernatant, pellets were solubilized with percholoric acid and the Ca²⁺ content was determined by atomic absorption spectroscopy. For these experiments, the EGTA concentration was reduced to 1 mM and FITC labeled dextran was present to mark the extramitochondrial volume with a fluorescent indicator. Fluorescence associated with mitochondrial pellets was then also determined and used to correct the atomic absorption measurements for extramitochondrial Ca²⁺.

Other Methods and Materials.

Protein concentrations were determined as absorbance of SDS solubilized mitochondria, using a value of A₂₈₀=0.205 at 10 mg protein/ml to standardize the measurements (Yaffe, M. P., 1991, Analysis of Mitochondrial Function and Assembly, Academic Press, NY). Fumarase and glucose-6-phosphate dehydrogenase activities were taken as markers for the mitochondrial matrix space and the cytoplasmic space, respectively (Zinser et al., 1995 Yeast 11:493). These values were used to estimate the fraction of mitochondria that are disrupted during the disruption of spheroplasts so that the fraction of aequorin associated with mitochondria in vivo could be determined. Briefly, the enzymes were assayed as described (Bergmeyer, 1974, Fumarase, in Methods of Enzymatic Analysis, Academic Press, NY; Bergmeyer, 1974, Glucose-6-Phosphate Dehydrogenase in Methods of Enzymatic Analysis, Academic Press, NY), using mitochondrial, microsomal, and cytoplasmic fractions obtained by differential centrifugation from spheroplast homogenates. The samples were treated with 1% Triton X-100™ to solubilize membrane-bound components and compartments prior to measuring enzyme activity.

Coelenterazine and its synthetic analogs were obtained from Biosynth International (Naperville, Ill.) or Molecular Probes (Eugene, Oreg.) and were dissolved in methanol. A stock solution of standard CaCl₂ was purchased from Orion (Boston, Mass.). Other compounds were from commercial sources and were reagent grade or better.

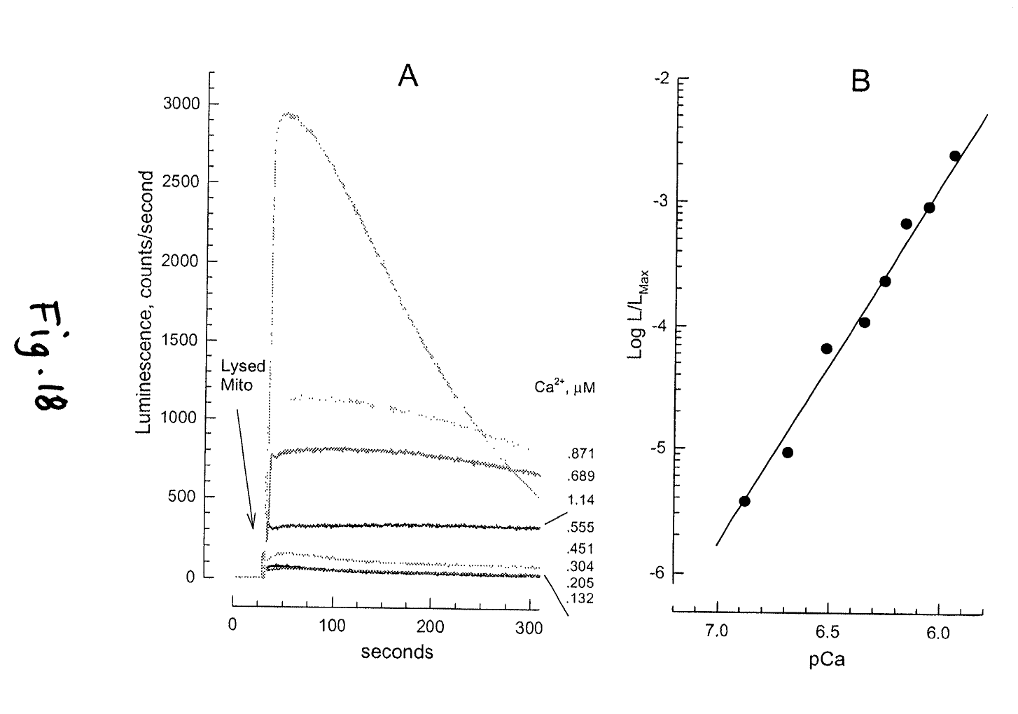

FIG. 18 shows calibration data obtained with lysed mitochondria at free Ca2+ concentrations spanning the range used in this example. In FIG. 18A, mitochondria were lysed with Triton X-100™ and luminescence was determined as described above. Numbers associated with the individual traces correspond to buffered Ca²⁺ concentrations which were established by adding CaCl₂ to the standard medium. In sequence, the levels required were 0.56, 0.75, 0.94, 1.12, 1.22, 1.31, 1.41, and 1.50 mM CaCl₂. For each trace, background counts were recorded for thirty seconds before the addition of lysed mitochondria. In FIG. 18B; log L/Lₘₐₓ values were obtained from the traces of FIG. 18A and plotted vs. pCa. These values were calculated at 100 seconds, or sooner, and were based upon Lₜₒₜₐₗ=1.34×106. The latter value was determined as a mean from three separate samples which contained the same amount of lysed mitochondria used for the other groups, with 20 μM free Ca²⁺ present to promptly discharge all of the aequorin The solid line in this panel was obtained by fitting the individual points to a linear model.

As shown in FIG. 18B, the relationship between the free Ca²⁺ concentration and luminescence intensity can be represented as a straight line, with the practical limit for detection of free Ca²⁺ around 10⁻⁷ M.

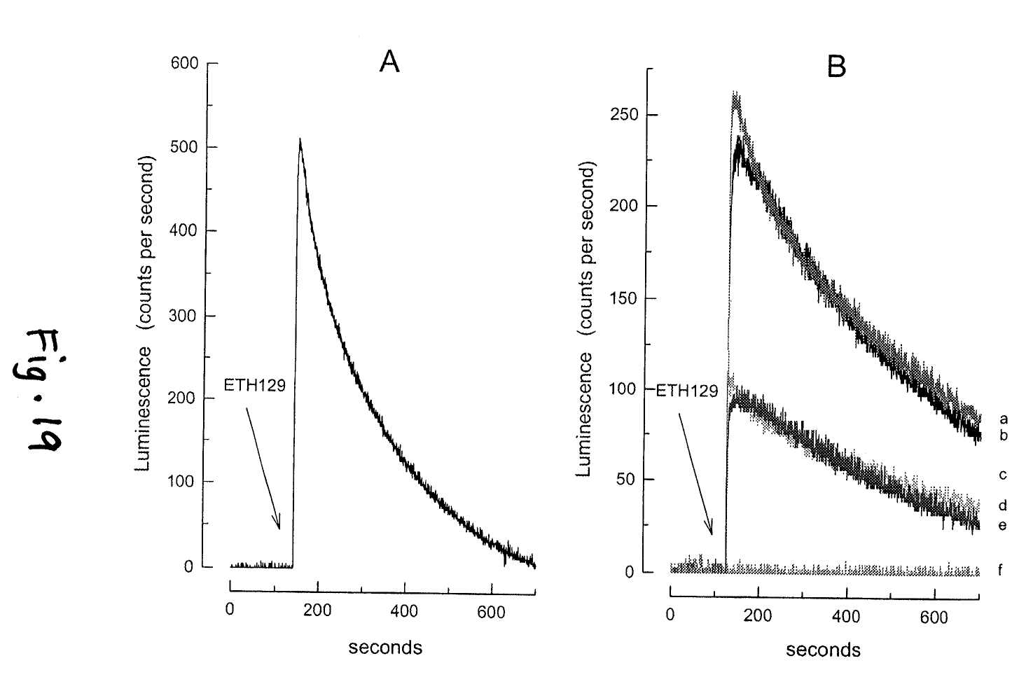

Since FIG. 18 was obtained using lysed mitochondria, it is apparent that the leader sequence from human cytochrome oxidase subunit VIII directed the aequorin apoprotein to the yeast organelle. FIG. 19 shows that the reconstituted apoprotein was only weakly luminescent in intact yeast which were oxidizing ethanol, but was strongly activated upon the addition of ionophore ETH 129. In FIG. 19A, cells were harvested at A₆₀₀=1.7, sedimented by centrifugation for 5 min at 3200× g, washed with distilled/deionized water, and resuspended in 1.2 M sorbitol, 20 mM KH₂PO₄, pH 7.0. Intracellular aequorin was formed from the apoprotein by incubating 0.3 ml of the cell suspension with 3 μl of a 0.25 mM coelenterazine solution, for 3 hr, while standing on ice. Subsequently 10 μl of the suspension was added to 0.3 ml of distilled/deionized water containing 1.4 mM ethanol (as repiratory substrate), 14 mM CaCl₂, and luminescence was monitored as described above. At 120 seconds, 6.5 μM of ionophore ETH 129 was added as indicated in the figure. Conditions in FIG. 19B were the same as described for FIG. 19A except that various coelenterazine analogs were used to reconstitute the apoprotein and ethanol was not present in the final suspension. The analogs employed were as follows, using the nomenclature of Shimomura etal. (1993 Cell Calcium 14:373); trace a (compound hcp), trace b (parent compound), trace c (compound f), trace d (compound h), trace e (compound n). Trace f is the control experiment, in which only the solvent used to prepare cofactor stock solutions (methanol) was added.

As noted above, ETH 129 is an electrogenic ionophore for Ca²⁺ that transports at a rate that is highly dependent on membrane potential. This ionophore provoked discharge of essentially all of the aequorin in intact cells, as indicated by the absence of any additional luminescence when ionophore A23 187, which transports Ca²⁺ by a charge-independent mechanism (Erdahl et al., 1994 Biophys. J. 66:1678; Thomas et al., 1997 Arch. Biochem. Biophys. 342:351) and therefore raised the Ca²⁺ concentration in all subcellular compartments, was added after the response to ETH 129 was complete. Little or no apoaequorin thus appeared to be present in non-mitochondrial, particulate cellular structures.

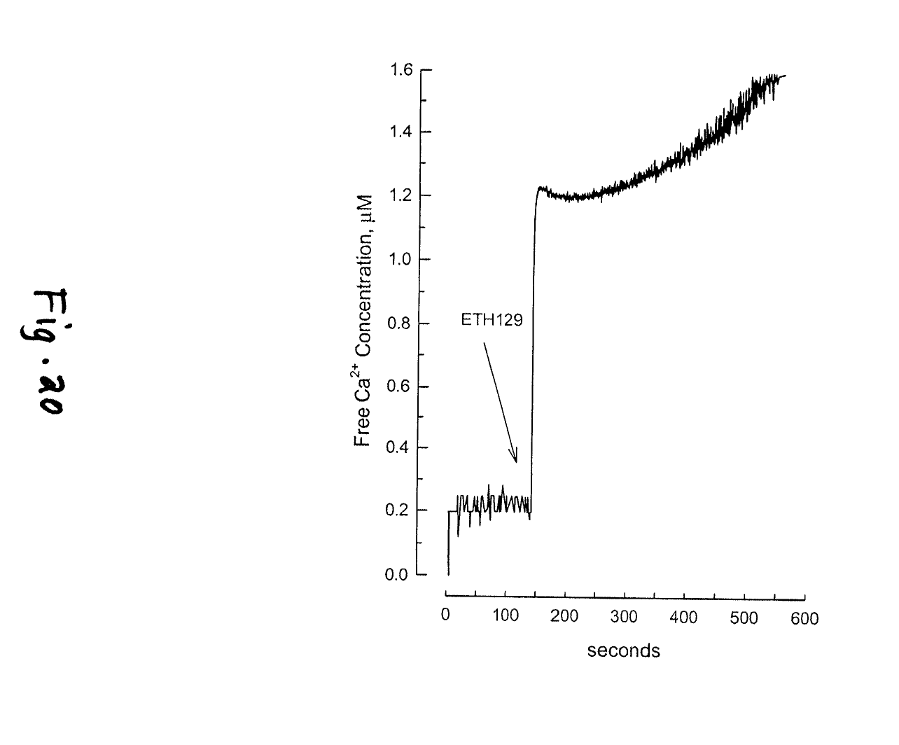

To determine if cytoplasm contained a significant fraction of the functional divalent cation-sensitive indicator molecule aequorin, total luminescence was compared in particulate and soluble fractions obtained from a high-speed centrifugation of disrupted cells. Approximately 20% of the activity was obtained in the soluble fraction, as was a similar fraction of fumarase activity, which reflected the fraction of mitochondria that have been disrupted. Taken together with the data obtained using ionophores, this finding indicated that the fraction of recombinant protein that was located in mitochondria approached 100%. Several coelenterazine derivatives were also evaluated as potential substitutes for the parent aequorin cofactor. Of the derivatives tested, only coelenterazine n produced intensity comparable to that of the parent, coelenterazine, when intact cells were tested (FIG. 19B), and similar results were obtained with isolated mitochondria. Ionophore dependent active accumulation and the retention of Ca²⁺ by yeast mitochondria are shown in FIG. 20. Calibration of the data shown in FIG. 19A according to the procedures described above indicated that the Ca²⁺ concentration was ˜200 nM in cells that were oxidizing ethanol, that it rose by 6-fold in response to ETH 129, and that a slowly increasing value was maintained for several minutes thereafter (FIG. 20).

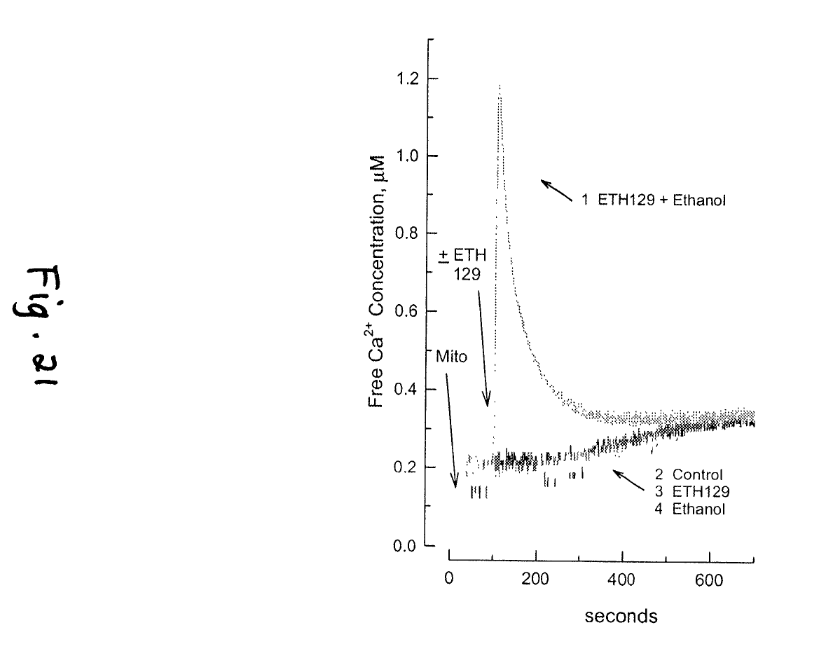

FIG. 21 shows luminescence properties of isolated yeast mitochondria. Mitochondria were incubated in the KCl-based standard medium described above, with 1.12 mM CaCl₂ present to give a free Ca²⁺ concentration of 0.45 μM. When utilized, the concentrations of ethanol (present from time 0) and ETH 129 (added at 120 seconds) were 0.5 mM and 15 μM, respectively. For trace 1, ethanol and ETH 129 were present; for trace 2; ethanol and ETH 129 were not present. For trace 3; ETH 129 only was present, and for trace 4 only ethanol was added. Under similar conditions with respect to ethanol, free Ca²⁺, and ionophore concentrations, isolated yeast mitochondria showed an analogous rise in the matrix free Ca²⁺ concentrations when ionophore was added, however, the elevated values were not maintained over time (FIG. 21). Increasing or decreasing the concentration of ETH 129 produced parallel changes in the maximal value but did not effect the decline, or the final value that was ultimately attained (not shown).

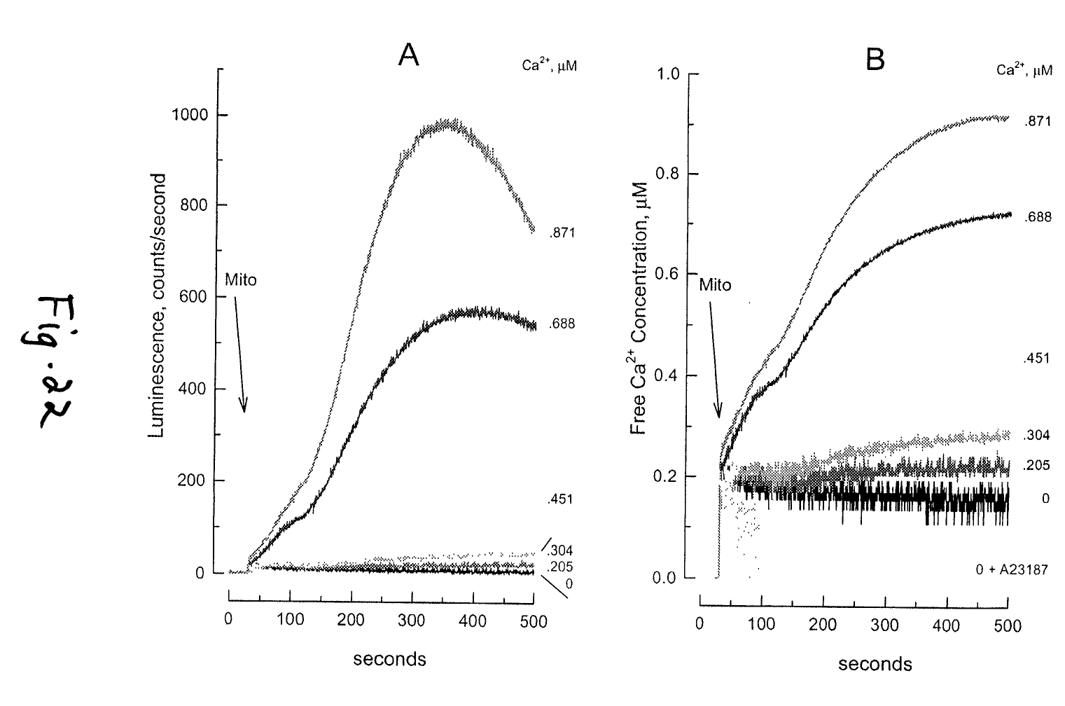

Passive accumulation and release of Ca²⁺ through an endogenous mitochondrial activity were also detected in yeast mitochondria (FIG. 22). Mitochondria were incubated in the KCl-based standard medium described above, without ethanol and without ETH 129. CaCl₂ was present at various concentrations to give the medium free Ca2+ concentrations specified, in units of μM, next to the individual recordings in both panels of FIG. 22. FIG. 22A shows luminescence plotted as a function of time, and in FIG. 22B, these data have been converted into units of matrix free Ca²⁺ concentration using the calibration procedure described above. FIG. 22B also shows that 2 μM A23187 reduced the matrix Ca²⁺ concentration below the detection limit when it was utilized in a medium containing no added Ca²⁺.

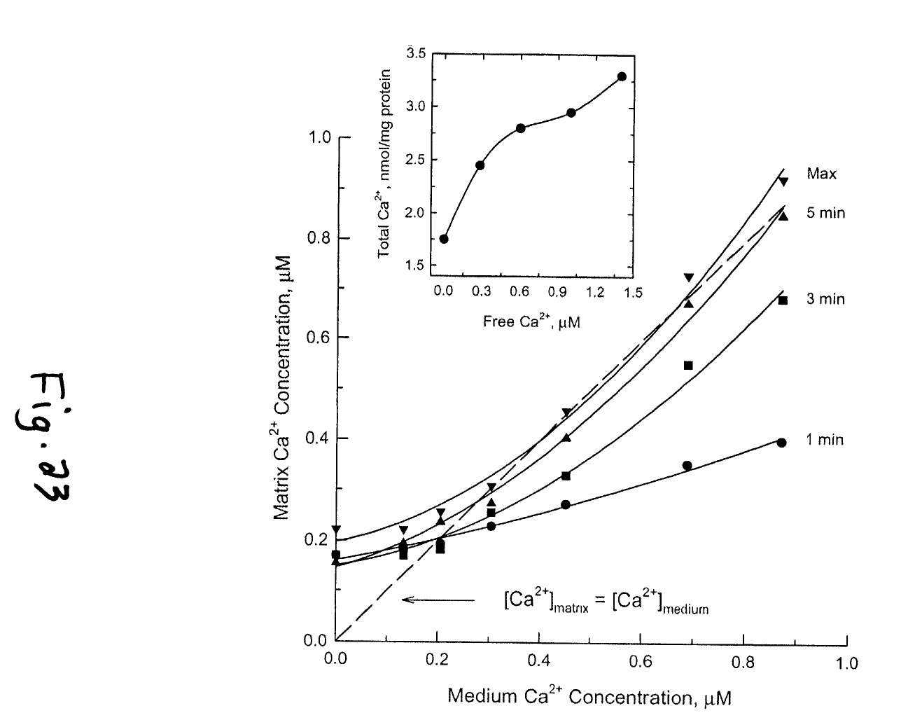

Plots of the medium free Ca²⁺ concentration against the matrix free Ca²⁺ concentration after various periods of incubation showed that equilibration was attained on a time scale of approximately 5 minutes (FIG. 23). Values were taken from FIG. 22B at 1, 3 and 5 minutes, and at the latest time point examined (11.6 minutes) when the extent of equilibration was maximal. The dashed line shown in the main panel is the line of equivalence between external and internal free Ca2+ concentrations. The inserted panel shows the relationship between matrix free Ca²+ concentration and the Ca²⁺ content of yeast mitochondria under the conditions used for the main panel. Each value was determined by atomic absorption spectroscopy following a 10 minute incubation, sedimenting the mitochondria in a microcentrifuge, and removing the resulting supernatant. Values were corrected for Ca²⁺ in the remaining extramitochondrial volume, which was determined by labeling with FITC dextran. The inserted panel in FIG. 23 shows the relationship between the matrix free Ca²⁺ concentration and total Ca²+in yeast mitochondria. The activity coefficient was much larger than in mammalian mitochondria (cf, e.g., Gunter et al., 1990 Am. J. Physiol. 258:C755-C786 for review), such that Ca²⁺ loads near 3 nmol/mg protein were sufficient to produce a free concentration in excess of 1 μM. From these data and those of FIG. 22, specific activity of the transporter mediating Ca²⁺ entry was estimated to be on the order of 0.2 nmol/minute/mg protein.

Activity of the transporter in yeast mitochondria was not influenced by ruthenium red, the uncoupler FCCP, or by nigericin and valinomycin, which are electroneutral and electrogenic ionophores for K+, respectively (table 1). Mitochondrial Ca²⁺ accumulation thus appeared not to generate, and was not effected by, electrochemical gradients of H⁺ or K⁺.

TABLE 2
Effects of uncoupler, nigericin, and valinomycin on Ca²⁺ transport in
isolated yeast mitochondria^{a}

| | Rate of increase in matrix | |
|---|---|---|
| | Ca²⁺ concentration, | |
| Condition | nM/second | % of Control |
| Control | 1.96 ± .08 | 100 |
| Ruthenium red | 2.21 ± .23 | 113 |
| FCCP | 2.16 ± .13 | 110 |
| Nigericin | 2.17 ± .12 | 111 |
| Valinomycin | 2.32 ± .31 | 118 |

^{a}Values were determined in triplicate at an external free Ca²⁺ concentration of 0.8 μM, while ruthenium red, FCCP and the ionophores nigericin and valinomycin were used at a concentration of 1 μM.

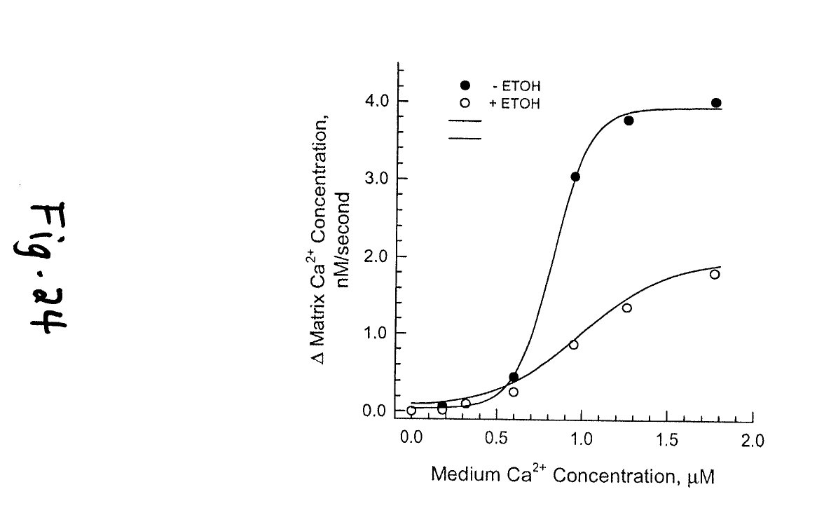

FIG. 24 shows the rate of change in the mitochondrial matrix Ca²⁺ concentration as a function of the external concentration. Mitochondria were added to the standard medium containing a range of buffered Ca²⁺ concentrations and Ca²⁺ transport was monitored as described above for FIG. 22. Where indicated, 0.5 mM ethanol was present as an oxidizable substrate. Initial rates, in units of nM matrix Ca²⁺ concentration×s⁻¹, were determined by linear regression analysis of the first 100 second portion in the progress curves. The resulting values were fit to the expression for a sigmoidal curve, which yielded half-maximal values at 0.82 and 1.0 μM Ca2+ for nonrespiring and respiring mitochondria, respectively; the maximal rate of change was reduced by a factor of ˜2 in mitochondria that were oxidizing ethanol.

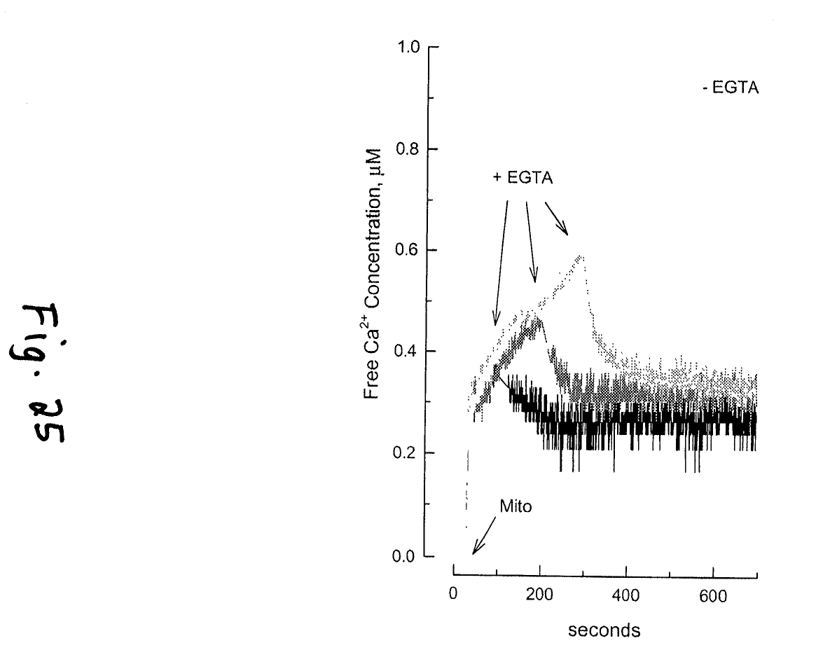

Ca²+ release from previously loaded yeast mitochondria is depicted in FIG. 25. The standard medium was employed with ethanol absent, using a buffered Ca²⁺ concentration that was initially near 0.8 μM. After mitochondria had accumulated Ca²⁺ for various lengths of time, the medium concentration was reduced to 0.15 μM by adding 2.5 mM of additional EGTA and the release of Ca²⁺ was monitored as shown.

### Example 4

### Expression Cloning of Mitochondrial Calcium Uniporter Polypeptide

A Saccharamyces cerevisiae yeast strain that expresses the divalent cation-sensitive indicator molecule recombinant aequorin targeted to mitochondria by the leader sequence from subunit VIII of human cytochrome c oxidase is prepared as described above, and is suitable for use in cloning Ca²⁺ transporters from mammalian mitochondria by expression-based methods. Using standard transfection methods such as those described in Example 3, a nucleic acid expression construct comprising a promoter operably linked to a nucleic acid encoding a candidate mitochondrial calcium uniporter polypeptide, which nucleic acid is derived from a human cDNA library, is contacted with the recombinant yeast under conditions and for a time sufficient to permit expression of the candidate uniporter. The cells are incubated with coelenterazine (2.5 μM) and 2-mercaptoethanol (2 μl), for 0.5-3 hr while standing on ice, to form the luminescent aequorin from its apoprotein as described in Example 3, washed and placed in warm medium containing 0.1 μM Ca²⁺, and the basal rate of light emission from such cells is recorded using a luminometer, as also described in Example 3.

The medium is replaced with fresh medium containing increasing Ca²⁺ concentrations, with luminometer readings taken following each increase in concentration to detect a signal generated by reconstitution of the mitochondrial, divalent cation-sensitive apoaequorin indicator molecule with Ca²⁺. Positive control cells are treated with 6.5 μM ETH 129 and methanol to initiate energy dependent Ca²⁺ accumulation in mitochondria, as described above. Cells that exhibit luminescence indicative of Ca2+ transport into mitochondria are isolated and expanded in growth cultures for expression and further characterization of the expressed candidate mitochondrial divalent cation transporter polypeptide (e.g., candidate calcium uniporter) responsible for Ca²⁺ translocation into the mitochondria. Detection of light emission and separation of cells generating a photosignal due to reconstitution of aequorin with Ca²⁺ may optionally be conducted using a fluorescence activated cell sorter such as a Coulter EPICS™ (Coulter Instruments, Hialeah, Fla.) or a Becton Dickinson FACStar™ (Becton Dickinson, San Jose, Calif.) instrument according to the manufacturer's instructions.

Mitochondria derived from recombinant yeast strains expressing an autheptic mitochondrial calcium uniporter exhibit (i) a capacity to transport Ca²⁺, Sr²⁺ and Mn²⁺ through an electrogenic mechanism that is inhibited by ruthenium red/ruthenium 360, lanthanide series trivalent cations, and by hexamine cobalt; (ii) mitochondrial Ca²⁺ transport kinetics that are a function of Δψ, such that the rate increases and decreases with parallel changes in Δψ, while the external Ca²⁺ is held constant; and (iii) regulation through distinct mitochondrial external sites that bind divalent cations, adenine nucleotides and polyamines, as observed, for example, under conditions of reverse activity described above. Mitochondrial preparations for these purposes are as described above; alternatively, cultures may be transformed into spheroblasts for mitochondrial characterization according to established procedures (e.g., Sutton et al., 1961 Proc. Soc. Exp. Biol. NY 108:170; Manon et al., 1998 Biochem. Mol. Biol. Int. 44:565; Averet et al., 1998 Molec. Cell. Biochem. 184:67)

### Example 5

### Growth Sensitivity to Calcium in a Calcium Atpase-defective Yeast Mutant

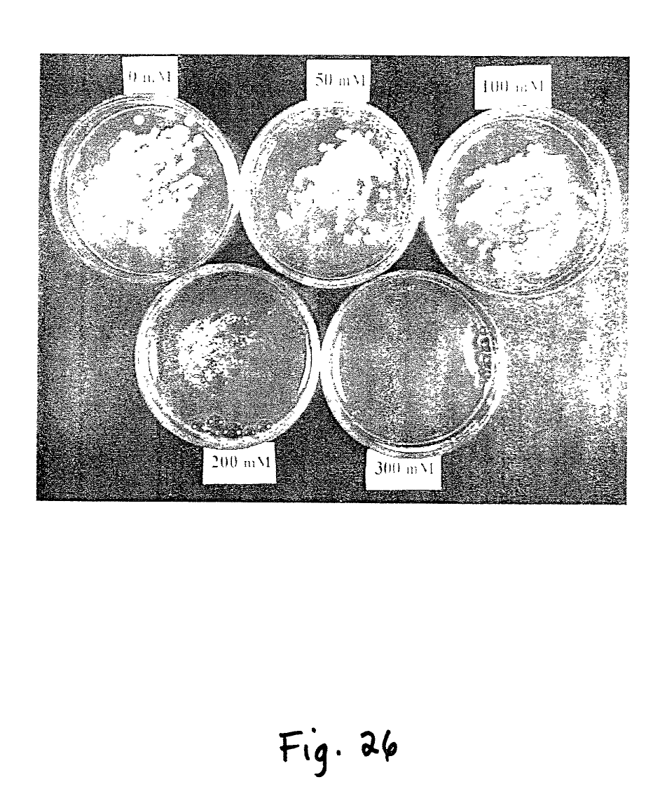

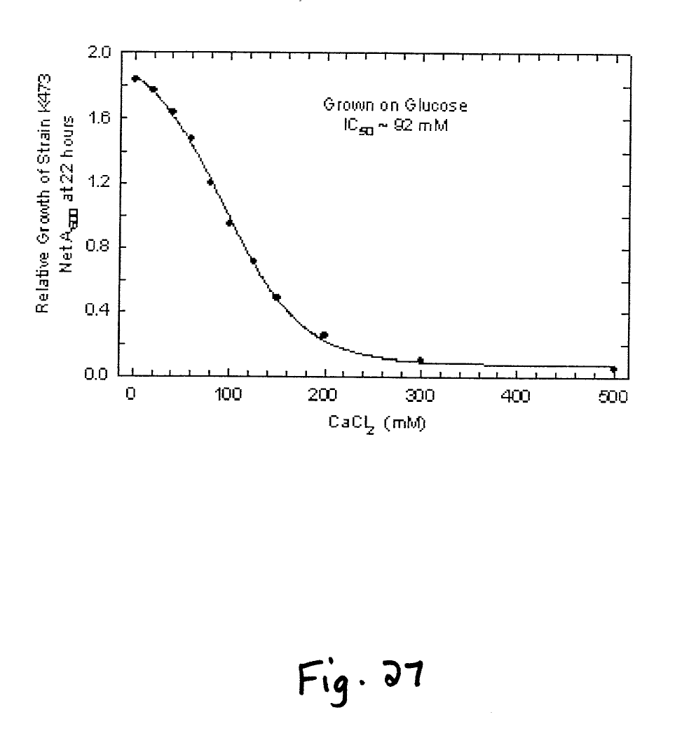

This example describes growth inhibition by Ca²⁺ of Saccharomyces cerevisiae strain K473, a pmc1 mutant lacking function in a vacuolar Ca2+ ATPase (Cunningham et al., 1996, Molec. Cell. Biology 16:2226-37; Cunningham et al., 1994 J. Cell Biol. 124:351-363). The strain was grown at 30° C. on standard YPD medium supplemented with 0, 50, 100, 200 or 300 mM CaCl₂, as indicated. Agar plates (FIG. 26) were incubated for 64 hrs. The growth curve (FIG. 27) determined after 22 hr indicated a 50% decrease in relative growth (IC50) at a calcium concentration of 92 mM. The IC50 for the wild type strain (W303-1A) is 360 mM Ca2+.

### Example 6

### Esterase Cleavage of Divalent Cation-sensitive Indicator Molecule Precursors

This example describes characterization of several bacterially expressed esterases according to their ability to cleave divalent cation-sensitive indicator molecule precursors to provide divalent cation-sensitive indicator molecules. Bacterial strains expressing Bacillus subtilis para-nitrobenzyl esterase [SEQ ID NOS:1 and 7] were obtained from the laboratory of Dr. F. H. Arnold (California Institute of Technology, Pasadena, Calif.) and were among those esterases that exemplify suitable esterases for use according to the present invention; such esterases have been previously described, for example, in U.S. Pat. Nos. 5,741,961; 5,906,930; 5,945,325; Zock et al., 1994 Gene 151:37-43; and Spiller et al., 1999 Proc. Nat. Acad. Sci. USA 96:12305-10.

Materials and Methods:

Standard molecular biology procedures were used (Sambrook et al., 1989 Molecular Cloning: A Laboratory Manual, Cold Spring Harbor Laboratory Press, Cold Spring Harbor, N.Y.). All reagents were from Sigma (St. Louis, Mo.) unless otherwise indicated. E. coli cells expressing esterases from several sources: B. subtilis para-nitrobenzyl esterase from plasmid PNB106R (Zock et al., 1994) (SEQ ID NO:1; SEQ ID NO:7); Acinetobacter areA expressed in E. coli strain BL21(DE3) (SEQ ID NO:2; from plasmid pADPW40, Jones et al. 1999 J. Bacteriol. 181:4568, obtained from Drs. R. Jones and P. Williams, Univeristy of Wales Bangor, UK); Acinetobacter SalE expressed in E. coli strain BL21(DE3) (SEQ ID NO:3; from plasmid pADPW70, Jones et al. 2000 J. Bacteriol. 1832:2018, also obtained from Drs. R. Jones and P. Williams, Univeristy of Wales Bangor, UK) were grown in 10 mL of standard LB media plus ampicillin (100 μg/ml) at 37° C. Tetracycline (10 μg/ml) was present instead of ampicillin for the cells expressing B. subtilis para-nitrobenzyl esterase. For strains expressing all proteins except para-nitrobenzyle esterase, the cells were grown to an OD (λ=600 nm) of 0.6 and protein expression was induced by administration of 0.4 MM IPTG for 4 hours until the culture reached an OD₆₀₀ of ˜1.0. Cells expressing para-nitrobenzylesterase were grown at 30° C. and protein expression was induced by a temperature shift to 40° C. for 6 hours. The cells attained an OD₆₀₀ of ˜1.0. Cells were washed, pelleted by centrifugation, and then resuspended in 0.5 ml of cold 0.12 M Tris Cl, pH 8.0. Cells were sonicated for ten seconds and then incubated on ice for ten seconds. This sonication step was repeated eight times.

For fluorescence measurements, a volume of 50 μl of the sonicated cell suspension was placed in 3.0 ml of 0.1 M KCl, 50 uM EGTA, 20 mM HEPES pH 7.40. As a divalent cation-sensitive indicator molecule, one of the following compounds (all from Molecular Probes, Inc., Eugene, Oreg.) was present at a final concentration of 1 μg/ml: BCECF AM (BCECF (2′,7′-bis-(2-carboxyethyl)-5-(and-6)-carboxyfluorescein), a proton indicator used as an esterase-cleavable control), Rhod-2 AM, Fluo-3 AM, Indo-1 AM, or Fura-2 AM. To monitor detection of divalent cation using the divalent cation-sensitive indicator molecules generated by esterase cleavage of the precursor forms, each reaction was made 2 mM CaCl₂ The wavelengths used for the indicator dyes were as follows: Fluo-3, excitation 488 nm, emission 520 nm; Rhod-2, excitation 540 nm, emission 580 nm; Indo-1 AM, excitation 338 nm, emission scan 350-600 run; Fura-2, excitation scan 250-450 nm, emission 510 nm; BCECF, excitation 440 nm, emission 550 nm.

Results:

Para-nitrobenzyl esterase from Bacillus subtilus detectably hydrolyzed all divalent cation-sensitive indicator molecule precursors that were tested, to provide divalent cation-sensitive indicator molecules having the expected Ca²⁺-sensitive fluorescent properties. Hydrolysis by areA from Acinetobacter of rhod-2 AM and fluo-2 AM to provide divalent cation-sensitive indicator molecules was also observed, although the rate of hydrolysis appeared to be significantly slower. Slow hydrolyses of Fura-2 AM and Indo-1 AM were also detected using HD-1 esterase from an oil degrading bacterium. (SEQ ID NO:4; Mizuguchi et al., 1999 J. Biochem. 126:731; obtained from Dr. S. Kanaya, Osaka University, Osaka, Japan, and subcloned using pBluescriptKS+ in E.coli strain DH5-α) All three of these esterases rapidly cleaved at least one acetoxymethylester group of the indicator molecule precursors to increase the apparent fluorescence of the precursors; however the indicator precursors were not converted to Ca²⁺ sensitive indicator molecules until a time period had elapsed that was approximately 30 to 60 fold longer than that required for the initial acetoxymethylester cleavage step. When Indo-1 AM was incubated with the SalE esterase of Acinetobacter no apparent Ca²⁺ sensitivity was detected even after a time period 60-fold longer than that required for the initial acetoxymethylester cleavage step had elapsed; this initial cleavage step increased fluorescence intensity of the precursor but did not result in conversion to a divalent cation-sensitive indicator molecule, as shown by an inability to respond to added Ca²⁺. All of the esterases that were tested rapidly cleaved the pH probe BCECF-AM, as evidenced by a shift in the peak fluorescence intensity of this probe.

### Example 7

### Transformation of Yeast Using B. Subtilis Para-nitrobenzyl Esterase

This example describes transformation of yeast with a nucleic acid expression construct, ymtPNB/pGK, encoding the B. subtilis para-nitrobenzyl esterase described in Example 6 (SEQ ID NO:1, derived from PNB106R, Zock et al. 1994 Gene 151:37), for expression as a fusion protein bearing the mitochondrial targeting sequence of human cytochrome c oxidase subunit VIII; the plasmid containing the sequence encoding the N-terminal targeting region from subunit VIII of human cytochrome c oxidase for use in preparing fusion constructs (Rizzuto et al., 1995 Meths. Enzymol. 260:417) is described above in Example 3. Standard molecular biology procedures were used (Sambrook et al., 1989 Molecular Cloning: A Laboratory Manual, Cold Spring Harbor Laboratory Press, Cold Spring Harbor, N.Y.). All reagents were from Sigma (St. Louis, Mo.) unless otherwise indicated. The B. subtilis para-nitrobenzyl esterase (PNB esterase) described in Example 6 was cloned into a yeast expression vector as follows:

The full-length B. subtilis PNB esterase encoding cDNA (SEQ ID NO:1) was amplified from the E. coli strain in which esterase activity was demonstrated in Example 6, by polymerase chain reaction (PCR) using total DNA from the esterase expressing bacteria as a template and Advantage™ cDNA Polymerase (Clontech, Palo Alto, Calif.) with standard cycling conditions. The 5′ forward oligonucleotide primer had a yeast mitochondrial targeting sequence and a BamH I restriction site incorporated into it, and the following sequence:

| | |
|---|---|
| 5′—fwd: | |
| 5′—ATTAGGATCCATGCTTTCACTACGTCAATCTATAAGATTTTTCAAGACTCAT | SEQ ID NO:5 |
| CAAATAGTAACGACT—3′ | |

The 3′ reverse oligonucleotide primer included an EcoR I restriction site and had the following sequence:

| | |
|---|---|
| ′—rev: | |
| 5′—TAATGAATTCTTATTCTCCTTTTGAAGGGAATAGCTT—3′ | SEQ ID NO:6 |

The PCR amplification product was digested with the BamH1 and EcoRI restriction enzymes, gel purified and ligated into the pGK (Brunelli et al., 1993 Yeast 9:1299) yeast expression vector (BamHI and EcoR I sites). Top 10™ E. coli cells (Invitrogen, Carlsbad, Calif.) were transformed with the ligation mixture and plated onto LB amp plates. Positive colonies were analyzed by restriction digestion and confirmed by fluorescence-based nucleotide sequence analysis using an ABI 3700 DNA sequencer according to the manufacturer's instructions (Applied Biosystems Division of Perkin-Elmer, Inc., Foster City, Calif.). Purified plasmid DNA (designated mtPNB/pGK) was used to transform the INV yeast strain (Invitrogen) and positive transformants were selected on the basis of their ability to grow on tryptophan-minus (Trp⁻) media. The transformed yeasts were tested for their ability to cleave the esters of Indo-1 (Indo-1 AM), BCECF (BCECF AM), and Fura 2 (Fura-2 AM) (all from Molecular Probes, Inc., Eugene, Oreg.), essentially according to the method described above in Example 6.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for identifying a nucleic acid molecule encoding a mitochondrial divalent cation transporter polypeptide, comprising:
a) contacting a biological sample comprising a host cell comprising at least one mitochondrion with at least one nucleic acid expression construct under conditions and for a time sufficient to permit expression of at least one mitochondrial divalent cation transporter polypeptide, wherein (i) the mitochondrion comprises a divalent cation-sensitive indicator molecule that is capable of generating a detectable signal in the presence of a divalent cation, and (ii) said nucleic acid expression construct comprises a promoter operably linked to a nucleic acid encoding a candidate mitochondrial divalent cation transporter polypeptide;
b) exposing the host cell to a divalent cation under conditions and for a time sufficient to permit transport of the divalent cation across a membrane by the candidate mitochondrial divalent cation transporter; and
c) detecting a signal generated by the divalent cation-sensitive indicator molecule in at least one mitochondrion, and therefrom identifying a nucleic acid encoding a mitochondrial divalent cation transporter polypeptide.

2. The method of claim 1 wherein the divalent cation is selected from the group consisting of barium, calcium, cobalt, iron, a lanthanide series member, lead, magnesium, manganese, zinc and strontium.

3. The method of claim 1 wherein the divalent cation is calcium.

4. The method of claim 1 wherein the host cell is a prokaryotic cell.

5. The method of claim 1 wherein the host cell is a eukaryotic cell.

6. The method of claim 5 wherein the eukaryotic cell is a yeast cell.

7. The method of claim 6 wherein the yeast cell is selected from the group consisting of Saccharomyces cerevisiae, Schizosacchromyces pombe, Candida albicans and Pichia pastoris.

8. The method of claim 1 wherein the host cell mitochondrion lacks an endogenous electrogenic divalent cation transporter.

9. The method of claim 1 wherein activity of at least one endogenous gene product is substantially impaired, wherein said gene product is selected from the group consisting of an electrogenic divalent cation transporter and an electroneutral divalent cation transporter.

10. The method of claim 1 wherein the nucleic acid expression construct further comprises at least one additional polynucleotide that regulates transcription.

11. The method of claim 10 wherein the additional polynucleotide that regulates transcription encodes a repressor of said regulated promoter.

12. The method of claim 1 wherein the nucleic acid expression construct encodes a candidate mitochondrial divalent cation transporter polypeptide that is expressed as a fusion protein with a polypeptide product of a second polynucleotide.

13. The method of claim 12 wherein said fusion protein localizes to a cellular membrane.

14. The method of claim 13 wherein the cellular membrane is selected from the group consisting of a mitochondrial membrane, a vacuolar membrane, a vesicular membrane, an endoplasmic reticulum membrane, a Golgi membrane, a chloroplast membrane and a plasma membrane.

15. The method of claim 13 wherein the cellular membrane is a mitochondrial membrane.

16. The method of claim 15 wherein the mitochondrial membrane is an inner mitochondrial membrane.

17. The method of claim 1 wherein the divalent cation-sensitive indicator molecule is selected from the group consisting of an aequorin protein, luciferase, a green fluorescent protein or variant thereof, ⁴⁵Ca, rhod-2, fura-2, Indo-1, Fluo-3 and a FLASH sequence.

18. The method of claim 1 wherein the host cell mitochondrion comprises at least one esterase that is capable of cleaving a divalent cation-sensitive indicator molecule precursor to provide the divalent cation-sensitive indicator molecule.

19. The method of claim 1 wherein the host cell comprises at least one second nucleic acid expression construct which directs expression of an esterase that localizes to a mitochondrion, wherein the esterase is capable of cleaving a divalent cation-sensitive indicator molecule precursor to provide the divalent cation-sensitive indicator molecule.

20. The method of either claim 18 or claim 19 wherein the divalent cation-sensitive indicator molecule precursor is capable of crossing a cellular membrane.

21. The method of claim 20 wherein the cellular membrane is selected from the group consisting of a mitochondrial membrane, a vacuolar membrane, a vesicular membrane, an endoplasmic reticulum membrane, a Golgi membrane, a chloroplast membrane and a plasma membrane.

22. The method of either claim 18 or claim 19 wherein the esterase comprises a mitochondrial targeting sequence.

23. The method of either claim 18 or claim 19 wherein the divalent cation-sensitive indicator molecule precursor is an ester of a divalent cation-sensitive indicator molecule that is selected from the group consisting of Indo-1 and Fura-2.

24. The method of claim 1 wherein the mitochondrial divalent cation transporter polypeptide comprises a divalent cation uniporter polypeptide.

25. The method of claim 1 wherein the mitochondrial divalent cation transporter polypeptide comprises an electrogenic divalent cation transporter polypeptide.

26. The method of either claim 24 or claim 25 wherein the divalent cation uniporter is a calcium uniporter.

27. The method of claim 1 wherein the signal generated by the divalent cation-sensitive indicator molecule is detectable by a method selected from the group consisting of spectrophotometry, radiometry, fluorimetry, FRET and flow cytofluorimetry.

28. A method of identifying a nucleic acid encoding a mitochondrial divalent cation transporter polypeptide, comprising:
a) contacting a host cell with at least one nucleic acid expression construct under conditions and for a time sufficient to permit expression of at least one mitochondrial divalent cation transporter polypeptide, wherein (i) host cell growth is impaired in the presence of Ca²⁺, and (ii) said nucleic acid expression construct comprises a promoter operably linked to a nucleic acid encoding a candidate mitochondrial divalent cation transporter polypeptide;
b) exposing the host cell to a divalent cation under conditions and for a time sufficient to permit transport of the divalent cation across a membrane by the candidate mitochondrial divalent cation transporter; and
c) detecting cell growth in at least one host cell, and therefrom identifying a nucleic acid encoding a mitochondrial divalent cation transporter polypeptide.

29. The method of claim 28 wherein the host cell is a eukaryotic cell.

30. The method of claim 28 wherein the host cell is a prokaryotic cell.

31. The method of claim 29 wherein the host cell is a yeast cell selected from the group consisting of Sacchromyces cerevisiae, Sacchromyces pombe, Candida albicans and Pichia pastoris.

32. The method of claim 28 wherein the host cell comprises a mutated ATPase gene.

33. The method of claim 28 wherein the host cell comprises a vacuolar assembly mutation.

34. The method of claim 28 wherein the host cell comprises a yeast PMC1/PMR1 double ATPase mutant.

35. The method of claim 28 wherein the mitochondrial divalent cation transporter polypeptide comprises a uniporter.

36. The method of claim 35 wherein the uniporter comprises a calcium uniporter.

37. The method of claim 28 wherein the divalent cation is selected from the group consisting of barium, calcium, cobalt, iron, lead, a member of the lanthanide series, magnesium, manganese, zinc and strontium.

38. The method of claim 37 wherein the divalent cation is calcium that is exposed to the host cell at a concentration from about 0.01 μM to about 100 μM.

39. The method of claim 28 wherein cell growth is detected by a method selected from the group consisting of microscopy, enzyme activity, spectrophotometry, flow cytometry, fluorimetry, and luminometry.

40. A method of preparing a mitochondrial divalent cation transporter polypeptide, comprising culturing a host cell comprising a nucleic acid expression construct that encodes a protein comprising a candidate mitochondrial divalent cation transporter polypeptide identified according to the method of either claim 1 or claim 28, under conditions and for a time sufficient to permit expression of the polypeptide, and recovering the polypeptide.

41. The method of claim 40 wherein the host cell is a prokaryotic cell.

42. The method of claim 40 wherein the host cell is a eukaryotic cell.

43. The method of claim 42 wherein the host cell is a yeast cell selected from the group consisting of Sacchromyces cerevisiae, Schizosacchromyces pombe, Candida albicans and Pichia pastoris.

44. The method of claim 40 wherein the mitochondrial divalent cation transporter polypeptide comprises a uniporter.

45. The method of claim 44 wherein the uniporter comprises a calcium uniporter.


SEQUENCE LISTING (9 sequences)

SEQ ID NO: 1 - DNA, 1470 bp, Bascillus subtilis
atgactcatcaaatagtaacgactcaatacggcaaagtaaaaggcacaacggaaaacggcgtacataagtggaaaggcatcccctatgccaagccgcctgtcggacaatggcgttttaaagcacctgagccgcctgaagtgtgggaagatgtccttgatgccacagcgtacggtcctatttgcccgcagccgtctgatttgctctcactgtcgtatacagagctgccccgccagtccgaggattgcttgtatgtcaatgtatttgcgcctgacactccaagtcaaaatcttcctgtcatggtgtggattcacggaggcgctttttatcttggagcgggcagtgagccattgtatgacggatcaaaacttgcggcacagggagaagtcattgtcgttacattgaactatcggctggggccgtttggctttttgcacttgtcttcgtttgatgaggcgtattccgataaccttgggcttttagaccaagccgccgcgctgaaatgggtgcgggagaatatctcagcgtttggcggtgatcccgataacgtaacagtatttggagaatccgccggcggcatgagcattgccgcgctgctcgctatgcctgcggcaaaaggcctgttccagaaagcgatcatggaaagcggcgcttcccgaacaatgacaaaagaacaagcggcaagcactgcggctgcctttttacaggtccttgggattaatgagagccagctggacagattgcatactgtagcagcggaagatttgcttaaagcggccgatcagcttcggattgcagaaaaagaaaatatctttcagctgttcttccagcccgcccttgatccgaaaacgctgcctgaagaaccagaaaaatcgatcgcagaaggggctgcttccggcattccgctattgattggaacaacccgtgatgaaggatatttatttttcaccccggattcagacgttcattctcaggaaacgcttgatgcagcactcgagtatttactagggaagccgctggcagagaaagctgccgatttgtatccgcgttctctggaaagccaaattcatatgatgactgatttattattttggcgccctgccgtcgcctatgcatccgcacagtctcattacgcccctgtctggatgtaccggttcgattggcacccggagaagccgccgtacaataaagcgtttcacgcattagagcttccttttgtctttggaaatctggacggattggaacgaatggcaaaagcggagattacggatgaggtgaaacagctttctcacacgatacaatccgcgtggatcacgttcgctaaaacaggaaacccaagcaccgaagctgtgaattggccggcgtatcatgaagaaacgagagagacggtgattttagactcagagattacgatcgaaaacgatcccgaatctgaaaaaaggcagaagctattcccttcaaaaggagaataa

SEQ ID NO: 2 - protein, 326 aa, Acinetobacter sp.
MLQSLDPQLASFFAMLENQGAPAFDIGTLNDPNINEMRNWYLNFGPHVAGQFIELQDVEDLWIQVDDGSLRLRLYKTQRQHSESCETLIYFHGGGWCIGSIETHDLLCRHLVNDLDVNVISVDYRLAPEYPYPQAAHDAIHATQWIFEHASALGIHPDHLYVAGDSAGGNLAAITALTFKEQTYRTSLAGQILIYPCIDLTVIHDNKYPSFEKQKLTPPLSAEQMSKMIQIYLKNEDFAYQWLASPLLAQSHQHLPSTLIITAEFDPLHDQGCVYFEKLKSSLVNTEYHEIKGVVHGFFEMYGVLNKTKETINIIRAWLKNNTKSK

SEQ ID NO: 3 - protein, 239 aa, Acinetobacter sp.
MITYVLVHGSWHDGSLWEPVATHLRAQGHTVHCPTVAGHGPNADRNVTHAQCSQSIADYIVKHDLSEIVLLGHSYGGTIISKVAEAIPERIQRLIYWNAFVLQDGENMFDNMPEAYYELFTSLAAASGDNTVLLPYEVWRHAFINDADDQMAEETYKMLTPEPCQPFHDRLDLKKFYTLNIPKSYLNCTEDQALPAGFWHPKMSNRLGEFKLVEMGGSHEAMFTRPQELATKIIEASHD

SEQ ID NO: 4 - DNA, 954 bp, Unknown
  Sequence encoding petroleum-degrading bacterium esterase.
atgacactcgatgcccaggccaaggccattctcgaccagatcgctcgctcgccgatgcccaagctgcaccaggtgccggccagcgttgcccgccagatgttcgagatcagctgcaagctgaccgaaatcaagaacctgcccatcggccgggtcgaagaccgcgtcattccggggccggacgacacggaactgccgatccgcatctacacgcccgtcgcggcgccgcccgggccgctgcccgtgctggtcttcttccacggcggcggtttcgtcatcggcagcctcgacagccatgacgcgccctgccggctgatcgccaacgaagcgcgctgcctcgtcgtctcggtcgattatcgcctggccccggaaaaccgcttcccggcggcggtcgacgattgcctggccgcggtcacctgggtcgcccgcaacgccgccgagatcaacgccgacccgacccgcatcgccgtcggcggcgattccgccggcggcaacctctcggccgtggtctcccagcagttgcgcgacgcgggcgggccgaagatcgtcttccagttgctgatctatccggcgaccgacgccctgcacgaaggcctgtcgcggacctcgaacgccgaaggctacatgctggacaaggacctgatgtcctggttcttcgcgcagtatctgggcgacggcggcggcgtcgacctcgcggatccgcgcttctcgccgctgcgccatgccaatctcggcaatctgggcacgatccatgtcgtcgtcgccggcttcgacccgctgcgggacgaaggcatcgcctatgccgaggcactgaaggcggcgggcaacaaggtcacgctctccgagttcaagggccagatccacggcttctgctcgatggccggggtgatcgaggctggccgcaccgccctcgtcgaaggcgccgccctgctgaaggaagccttcgcccaggcctga

SEQ ID NO: 5 - DNA, 67 bp, Artificial Sequence
  PCR primer
attaggatccatgctttcactacgtcaatctataagatttttcaagactcatcaaatagtaacgact

SEQ ID NO: 6 - DNA, 37 bp, Artificial Sequence
  PCR primer
taatgaattcttattctccttttgaagggaatagctt

SEQ ID NO: 7 - DNA, 1470 bp, Bacillus subtilis; identical to SEQ ID NO: 1

SEQ ID NO: 8 - DNA, 72 bp, Artificial Sequence
  PCR primer
aaaagatctaaaaatgctttcactacgtcaatctataagatttttcaagcttacatcagacttcgacaaccc

SEQ ID NO: 9 - DNA, 29 bp, Artificial Sequence
  PCR primer
aaaggtaccttaggggacagctccaccgt